(19) United States
(12) Patent Application Publication
(10) Pub. No.: US 2026/0285816 A1
(43) Pub. Date: Sep. 24, 2026

(54) Piperazinyl-Modified Quinazolinyl-Aryl Urea Derivative, And Preparation Method And Use Thereof

(71) Applicant: Guangxi Normal University, Guilin (CN)

(72) Inventors: Jianian Chen, Guilin (CN); Haiheng Zeng, Guilin (CN); Wenting Xing, Guilin (CN); Zhiming Hu, Guilin (CN); Yixuan Ma, Guilin (CN); Yue Liu, Guilin (CN); Qiuchen Li, Guilin (CN); Zhihao Liang, Guilin (CN); Xiangyang Tong, Guilin (CN)

(73) Assignee: Guangxi Normal University, Guilin (CN)

(21) Appl. No.: 19/286,638

(22) Filed: Jul. 31, 2025

(30) Foreign Application Priority Data
Mar. 21, 2025 (CN) 202510337892.2

(51) Int. Cl. C07D 239/94; A61K 31/517; A61K 31/5377; A61P 35/00; C07D 401/14

(52) U.S. Cl. CPC C07D 239/94; A61K 31/517; A61K 31/5377; A61P 35/00; C07D 401/14

(57) ABSTRACT

Provided are a piperazinyl-modified quinazolinyl-aryl urea derivative, and a preparation method and use thereof. The piperazinyl-modified quinazolinyl-aryl urea derivative has a structure shown in Formula (I); where, n is in a range of 3 to 7; R₁ is selected from the group consisting of 4-(2-morpholinoethyl)piperazin-1-yl and 4-((4-(pyridin-2-yl)piperazin-1-yl)methyl)piperidin-1-yl, and R₂ is selected from the group consisting of phenyl, substituted phenyl, benzyl, and cycloalkyl; the substituted phenyl comprises a benzene ring and a substituent linked to the benzene ring, the substituent exhibits mono-substitution, identical multi-substitution, or different multi-substitution, and is at least one selected from the group consisting of alkyl, alkoxy, nitro, a halogen atom, and a halogenated group; and the cycloalkyl is selected from the group consisting of cyclopropyl, cyclobutyl, cyclopentyl, cyclohexyl, and cycloheptyl.

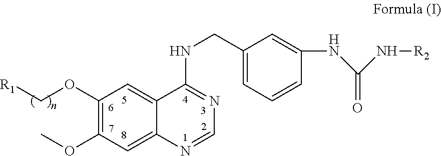

### CROSS REFERENCE TO RELATED APPLICATION

This patent application claims the benefit and priority of Chinese Patent Application No. 2025103378922 filed with the China National Intellectual Property Administration on Mar. 21, 2025, the disclosure of which is incorporated by reference herein in its entirety as part of the present application.

### TECHNICAL FIELD

The present disclosure relates to the technical field of medicine, and in particular relates to a piperazinyl-modified quinazolinyl-aryl urea derivative, and a preparation method and use thereof.

### BACKGROUND

In April 2024, the International Agency for Research on Cancer (IARC) team of the World Health Organization (WHO) published the 2022 Global Cancer Statistics Report in CA: A Cancer Journal for Clinicians, detailing the incidence and mortality of 36 malignancies across 185 countries or regions worldwide. In 2022, nearly 20 million new cancer cases and 9.7 million cancer-related deaths were recorded. Lung cancer has remained the most prevalent globally. Ranked 2nd to 10th in incidence are female breast cancer, colorectal cancer, prostate cancer, gastric cancer, liver cancer, thyroid cancer, cervical cancer, bladder cancer, and non-Hodgkin's lymphoma.

Bladder cancer is among the most common genitourinary malignancies. For early- to mid-stage cases, “surgical resection combined with intravesical chemotherapy” has become the primary treatment. However, due to the unique properties of bladder tissue, surgical resection is not the optimal choice for all patients. For advanced or metastatic bladder cancer, platinum-based combination chemotherapy remains the first-line regimen despite multiple therapeutic options. The platinum-based combination chemotherapy includes methotrexate+vinblastine+adriamycin (ADR)+cisplatin (MVAC), gemcitabine+cisplatin (GC), taxol+cisplatin (TC), taxol+carboplatin (TCa), and docetaxel+cisplatin (DC). In April 2019, the U.S. Food and Drug Administration (FDA) granted accelerated approval for erdafitinib (Erda, having a structure shown in Formula (II)), the first small-molecule targeted drug for metastatic bladder cancer. Erda is indicated for locally advanced or metastatic bladder cancer patients with FGFR3 or FGFR2 mutations who have experienced disease progression following platinum-based chemotherapy. Due to poor water solubility, only oral solid formulations are currently available for the Erda.

Osteosarcoma (OS), also known as osteogenic sarcoma, is a highly malignant bone tumor with complex etiology, characterized by aggressiveness and high lethality, showing an incidence of (3-5)/million. Current first-line chemotherapeutic agents for osteosarcoma primarily include doxorubicin (also known as ADR), cisplatin (CDDP), and methotrexate (MTX). However, due to the toxic side effects and drug resistance associated with chemotherapy, most patients are forced to discontinue treatment. Drug resistance poses a significant obstacle, particularly in treating advanced osteosarcoma. In recent years, tyrosine kinase inhibitors (TKIs) such as apatinib mesylate (Apat) and anlotinib hydrochloride have been applied in osteosarcoma treatment (the Apat has a structure shown in Formula (III)). Certain TKIs including gefitinib (Gef) and erlotinib hydrochloride (Erlo) can modestly enhance chemotherapy efficacy (the Gef has a structure shown in Formula (IV), while the Erlo has a structure shown in Formula (V)). Gef, a selective epidermal growth factor receptor tyrosine kinase inhibitor (EGFR-TKI), has demonstrated favorable efficacy in treating locally advanced or metastatic non-small cell lung cancer (NSCLC) in patients previously treated with chemotherapy or unsuitable for chemotherapy. Additionally, sorafenib (Sora) and regorafenib (Reg) have shown certain effectiveness in osteosarcoma therapy. Compared to everolimus monotherapy, Sora combined with everolimus exhibits advantages for unresectable high-grade osteosarcoma (the Sora has a structure shown in Formula (VI)). Reg, a structural analog (also known as a “Me-too” drug) of Sora, exerts antitumor effects by inhibiting molecular targets such as VEGFR1-3 (the Reg has a structure shown in Formula (VII)).

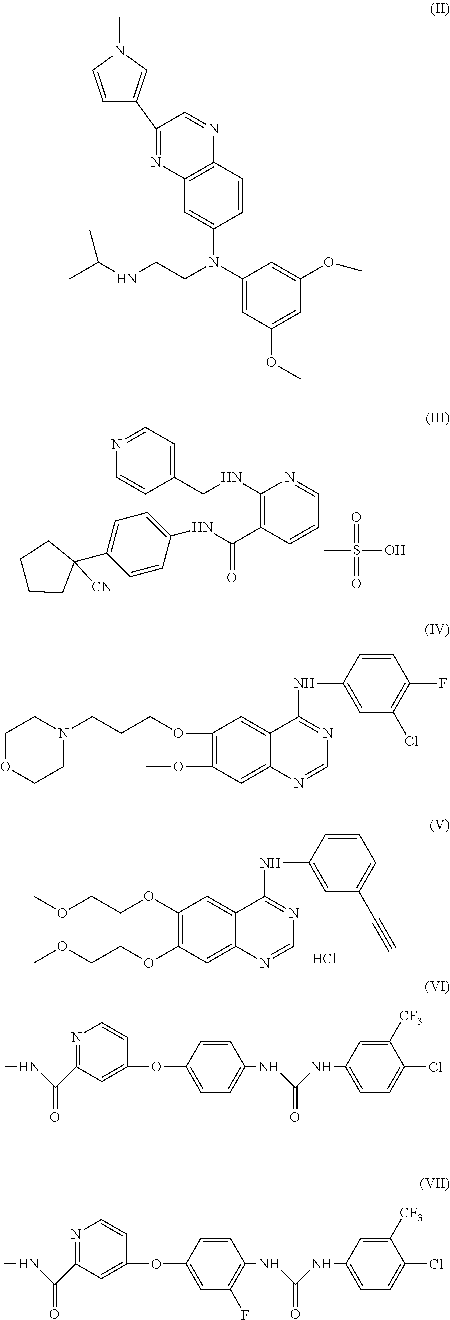

Pancreatic cancer (PAAD) is a highly refractory and aggressive malignancy characterized by insidious onset and rapid progression. With a five-year survival rate of approximately 10% post-diagnosis, the pancreatic cancer ranks among the malignancies with the poorest prognosis. Surgery-based comprehensive therapy remains the primary treatment approach, yet most pancreatic cancer patients are ineligible for resection at diagnosis. Current chemotherapeutic agents for unresectable locally advanced or metastatic pancreatic cancer include gemcitabine (Gem), nab-paclitaxel (PTX), 5-fluorouracil, CDDP, oxaliplatin, irinotecan, tegafur/gimeracil/oteracil, and capecitabine. Targeted agents include Erlo, everolimus, and sunitinib.

Molecular targeted therapy involves designing therapeutic agents that selectively bind to defined oncogenic targets at the molecular level. The therapeutic agents induce tumor cell death while minimizing toxicity to normal tissues and cells by specifically binding to carcinogenic sites after drug molecules enter the body. Aryl urea derivatives such as the marketed drugs Sora, Reg, linifanib, and tivozanib are widely utilized in anticancer drug design.

Additionally, the transport and metabolism of aryl urea derivatives in vivo are closely associated with lipid/water partition coefficient. The lipid/water partition coefficient critically determines multiple pharmacokinetic properties in biological systems, including: solubility in biological fluids, permeability, absorption rate via gastrointestinal administration, affinity for plasma and tissue proteins, and potential for sustained-release effects. For instance, Sora exhibits poor aqueous solubility, rapid elimination/metabolism, and low oral bioavailability. Certain compounds demonstrate promising activity in preliminary screening but fail in animal studies or clinical trials due to unreasonable lipid/water partition coefficient, inadequate bioavailability, and challenges in formulation development. Moreover, some compounds suffer from dual solubility limitations—poor solubility in both aqueous phase and lipid phase. For example, compounds which only dissolve minimally in highly specific organic solvents exhibit unfavorable absorption profiles. Therefore, structural optimization of aryl urea derivatives must address both hydrophilicity enhancement and lipophilicity modulation.

### SUMMARY

The present disclosure provides a piperazinyl-modified quinazolinyl-aryl urea derivative, and a preparation method and use thereof, designed for treating various tumors including bladder cancer, osteosarcoma, and pancreatic cancer. By enhancing both the lipophilicity and hydrophilicity of compounds, the piperazinyl-modified quinazolinyl-aryl urea derivative achieves improved bioavailability.

In order to solve the above technical problems, a first object of the present disclosure is to provide a piperazinyl-modified quinazolinyl-aryl urea derivative or a pharmaceutically acceptable salt thereof, the piperazinyl-modified quinazolinyl-aryl urea derivative having a structure shown in Formula (I):

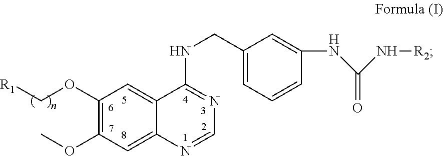

where
n is in a range of 3 to 7,
R₁ is selected from the group consisting of 4-(2-morpholinoethyl)piperazin-1-yl and 4-((4-(pyridin-2-yl)piperazin-1-yl)methyl)piperidin-1-yl, where the 4-(2-morpholinoethyl)piperazin-1-yl has a structure shown in

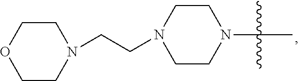 [SMILES: *N1CCN(CCN2CCOCC2)CC1]

and the 4-((4-(pyridin-2-yl)piperazin-1-yl)methyl)piperidin-1-yl has a structure shown in

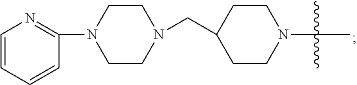 [SMILES: *N1CCC(CN2CCN(c3ccccn3)CC2)CC1]

and
R₂ is selected from the group consisting of phenyl, substituted phenyl, benzyl, and cycloalkyl; the substituted phenyl includes a benzene ring and a substituent linked to the benzene ring, the substituent exhibits mono-substitution, identical multi-substitution, or different multi-substitution, and is at least one selected from the group consisting of alkyl, alkoxy, nitro, a halogen atom, and a halogenated group; and the cycloalkyl is selected from the group consisting of cyclopropyl, cyclobutyl, cyclopentyl, cyclohexyl, and cycloheptyl.

In prior research, the inventor has employed a “pharmacophore hybridization” strategy by combining the carbamido group of Sora with the 4-aminoquinazolinyl group of Gef. This strategy creates quinazolinyl-aryl urea derivatives featuring a benzyl linker, yielding compounds I-1 to I-16 and II-1 to II-17, with a structure of general formula

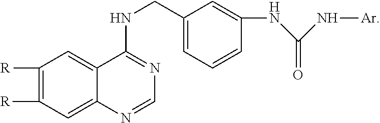

The compounds demonstrate antiproliferative activity against multiple tumor cell lines. However, human bladder cancer cells show sensitivity to most of the compounds, but the compounds suffered from suboptimal lipophilicity/hydrophilicity and unfavorable lipid-water partition coefficients. Structural optimization results in subsequently generated compounds 9a to 9y, with a structure of general formula

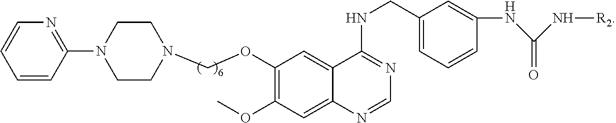

Yet limitations remain weak antiproliferative activity against hepatoblastoma and reduced efficacy toward lung cancer cells due to new modifiers. The present disclosure further discloses two novel series of piperazinyl-modified quinazolinyl-aryl urea derivatives. Series I introduces morpholinoethylpiperazine group at the quinazoline ring 6-position, yielding target compounds 8-1a to 8-1v. Series II features pyridylpiperazinylmethylpiperidine group at the quinazoline ring 6-position, yielding target compounds 8-2a to 8-2z. The target compounds show the following key advantages: potent antiproliferative activity against bladder cancer, hepatoblastoma, lung cancer, osteosarcoma, and pancreatic cancer cells; and significant tumor suppression in diverse tumor-bearing nude mouse models. The piperazinyl-modified quinazolinyl-aryl urea derivatives represent promising molecularly targeted antitumor agents. The target compounds in the present disclosure shows favorable solubility in multiple solvents, which enables injectable administration, oral delivery, and intravesical instillation (bladder perfusion).

In an embodiment, the alkyl in the substituent is at least one selected from the group consisting of methyl, ethyl, propyl, isopropyl, butyl, tert-butyl, pentyl, and isopentyl.

In an embodiment, the alkoxy in the substituent is at least one selected from the group consisting of methoxy, ethoxy, propoxy, isopropoxy, butoxy, and tert-butoxy.

In an embodiment, the halogen atom in the substituent is at least one selected from the group consisting of a fluorine atom, a chlorine atom, a bromine atom, and an iodine atom, and the halogenated group is trifluoromethyl.

In an embodiment, R₂ is selected from the group consisting of phenyl, 2-methylphenyl, 3-methylphenyl, 4-methylphenyl, 2,4-dimethylphenyl, 3,5-dimethylphenyl, 2-ethylphenyl, 2,6-diethylphenyl, 4-isopropylphenyl, 4-tert-butylphenyl, 2-methoxyphenyl, 4-methoxyphenyl, 4-fluorophenyl, 2-chlorophenyl, 4-chlorophenyl, 2,4-dichlorophenyl, 3,4-dichlorophenyl, 2,4,6-trichlorophenyl, 4-(trifluoromethyl)phenyl, (3-trifluoromethyl-4-fluoro)phenyl, (3-trifluoromethyl-4-chloro)phenyl, (3,5-bis(trifluoromethyl))phenyl, benzyl, and cyclohexyl.

In an embodiment, under a condition that R₁ in the Formula (I) is

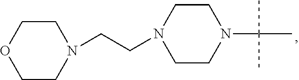

R₂ is selected from the group consisting of phenyl, 2-methylphenyl, 4-methylphenyl, 2-ethylphenyl, 2,6-diethylphenyl, 4-isopropylphenyl, 4-tert-butylphenyl, 4-fluorophenyl, 2-chlorophenyl, 3,4-dichlorophenyl, (3-trifluoromethyl-4-fluoro)phenyl, (3-trifluoromethyl-4-chloro)phenyl, 3,5-bis(trifluoromethyl)phenyl, and cyclohexyl.

In an embodiment, under a condition that R₁ in the Formula (I) is

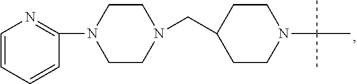

R₂ is selected from the group consisting of phenyl, 2-methylphenyl, 3-methylphenyl, 4-methylphenyl, 2,4-dimethylphenyl, 3,5-dimethylphenyl, 2-ethylphenyl, 2,6-diethylphenyl, 4-isopropylphenyl, 4-tert-butylphenyl, 2-methoxyphenyl, 4-methoxyphenyl, 4-fluorophenyl, 2-chlorophenyl, 4-chlorophenyl, 2,4-dichlorophenyl, 2,4,6-tri chlorophenyl, 4-(trifluoromethyl)phenyl, (3-trifluoromethyl-4-fluoro)phenyl, (3-trifluoromethyl-4-chloro)phenyl, (3,5-bis(trifluoromethyl))phenyl, benzyl, and cyclohexyl.

In an embodiment, the pharmaceutically acceptable salt is selected from the group consisting of a hydrochloride, a sulfate, a phosphate, a formate, an acetate, a nitrate, a benzoate, a citrate, a succinate, a palmitate (hexadecanoate), a stearate (octadecanoate), a besylate, a tartrate, a maleate, an oxalate, a malate, a lactate, a tosylate, a mesylate, an ethanesulfonate, a triflate, and a trifluoroacetate.

In order to solve the above technical problems, a second object of the present disclosure is to provide a method for preparing the piperazinyl-modified quinazolinyl-aryl urea derivative or the pharmaceutically acceptable salt thereof, including the following steps:
subjecting an intermediate 2 to demethylation with a demethylation reagent under heating to obtain an intermediate 3; where
the demethylation reagent includes concentrated sulfuric acid and L-methionine;
the intermediate 2 has a structure shown in

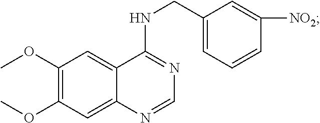 [SMILES: COc1cc2ncnc(NCc3cccc([N+](=O)[O-])c3)c2cc1OC]

and
the intermediate 3 has a structure shown in

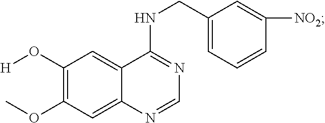 [SMILES: COc1cc2ncnc(NCc3cccc([N+](=O)[O-])c3)c2cc1O]

subjecting the intermediate 3, a haloalkyl compound, and K₂CO₃ to a substitution reaction under an inert gas atmosphere while a heating to obtain an intermediate 4; where
the haloalkyl compound has a structure shown in

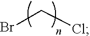

and
the intermediate 4 has a structure shown in

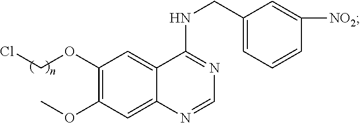

subjecting the intermediate 4, a substituted compound, K₂CO₃, and sodium iodide to a substitution reaction under an inert gas atmosphere while heating to obtain an intermediate 5; where
the substituted compound has a structure selected from the group consisting of

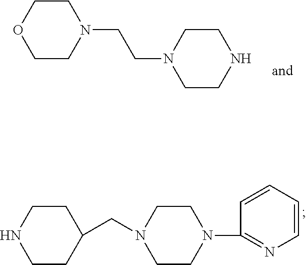 [SMILES: C1CN(CCN2CCOCC2)CCN1 ; c1ccc(N2CCN(CC3CCNCC3)CC2)nc1]

and
the intermediate 5 has a structure shown in

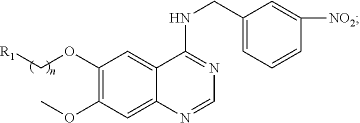

subjecting the intermediate 5 to nitro reduction with a reducing agent under heating to obtain an intermediate 6; where

the intermediate 6 has a structure shown in

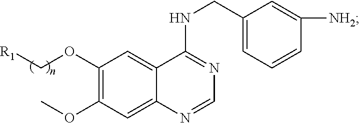

and
subjecting the intermediate 6 and an intermediate 7 to a reaction in the presence of a catalyst to obtain the piperazinyl-modified quinazolinyl-aryl urea derivative or the pharmaceutically acceptable salt thereof, where
the intermediate 7 has a structure shown in R₂—N═C═O; and
where n in the haloalkyl compound, the intermediate 4, the intermediate 5, and the intermediate 6 corresponds to n in Formula (I); R₁ in the intermediate 5 and the intermediate 6 corresponds to R₁ in Formula (I); and R₂ in the intermediate 7 corresponds to R₂ in Formula (I).

During the synthesis of intermediate 3, two methods are explored for efficient and selective removal of methyl at 6-position of intermediate 2:(1) a reaction is conducted while stirring by using methanesulfonic acid/L-methionine as a demethylation reagent at 110° C. for 12 h; (2) a reaction is conducted while stirring by employing a concentrated sulfuric acid solution/L-methionine as a demethylation reagent at 100° C. for 12 h. After experimentation, it has been found that Method (2) is significantly superior to Method (1) for the following reasons: Method (1) has yielded low conversion (<36%), producing a black mixture with substantial coal-tar-like viscous substances that complicated workup and isolation. Beyond the desired 6-mono-demethylated product, Method (1) yields 7-mono-demethylated (10%) and 6,7-di-demethylated (7%) byproducts alongside unidentifiable impurities. In contrast, Method (2) has achieved higher yield (72%) and enhanced selectivity, such as reducing 7-mono-demethylated and 6,7-di-demethylated byproducts to less than 5% each. Structural confirmation of the 6-demethylated product is established through comprehensive NMR (nuclear magnetic resonance) analysis, including ¹H NMR, ¹³C NMR, ¹H-detected heteronuclear multiple bond correlation (HMBC), and heteronuclear singular quantum correlation (HSQC) spectra. Validation data are detailed in the accompanying drawings for Method (2), proving that the 6-mono-demethylated product can be obtained efficiently by the Method (2).

In an embodiment, during the preparation of the intermediate 3, the intermediate 2 is prepared by a process including:
subjecting 4-chloro-6,7-dimethoxyquinazoline, 3-nitrobenzylamine hydrochloride, and N,N-diisopropylethylamine (DIPEA) to a reaction under a nitrogen atmosphere while heating to obtain the intermediate 2.

In an embodiment, during the preparation of intermediate 2, a molar ratio of the 4-chloro-6,7-dimethoxyquinazoline, the 3-nitrobenzylamine hydrochloride, and the DIPEA is in a range of 1:(0.7-1.1):(0.8-1.2). A reaction system further includes n-butanol as a solvent, and the reaction is conducted at a temperature of 100° C. to 130° C. for 20 h to 30 h.

In an embodiment, during the preparation of the intermediate 3, a molar ratio of the intermediate 2, the L-methionine, and the concentrated sulfuric acid is in a range of 1:(1.2-2.5):(12-25), and the demethylation is conducted at a temperature of 90° C. to 120° C. for 8 h to 16 h.

In an embodiment, during the preparation of intermediate 4, a molar ratio of the intermediate 3, the haloalkyl compound, and the K₂CO₃ is in a range of 1:(1-1.6):(0.8-1.2). A reaction system further includes acetonitrile as a solvent, and the substitution reaction is conducted at a temperature of 60° C. to 75° C. for 8 h to 32 h (16 h to 32 h in a further embodiment).

In an embodiment, during the preparation of intermediate 5, a molar ratio of the intermediate 4, the substituted compound, the K₂CO₃, and the sodium iodide is in a range of 1:(1-1.5):(0.8-1.2):(0.9-1.5). A reaction system further includes acetonitrile as a solvent, and the substitution reaction is conducted at a temperature of 80° C. to 90° C. for 40 h to 60 h.

In an embodiment, during the preparation of the intermediate 6, the reducing agent is selected from the group consisting of a combination of an activated iron powder and a 5 wt % to 20 wt % aqueous ammonium chloride solution and a combination of the activated iron powder and a 10 wt % to 20 wt % dilute hydrochloric acid, a molar ratio of the intermediate 5, the activated iron powder, and ammonium chloride is in a range of 1:(5-10):(1-2.5), a reaction system for the nitro reduction further includes methanol as a solvent, the nitro reduction is conducted at a temperature of 55° C. to 65° C. for 8 h to 16 h, and the activated iron powder is prepared by treating a reduced iron powder with a 10 wt % to 20 wt % aqueous hydrochloric acid solution.

The inventor has compared the following two reduction methods: (1) an activated iron powder combined with a 15 wt % aqueous hydrochloric acid solution; (2) an activated iron powder combined with a 10 wt % aqueous ammonium chloride solution. Method (2) demonstrates significant advantages over Method (1) for the following reasons: Method (1) requires cumbersome post-treatment and leads to substantial byproducts. Adjusting pH values consumes large quantities of base reagents, such as neutralizers for hydrochloric acid. By switching to Method (2), namely replacing dilute hydrochloric acid with a 10 wt % aqueous ammonium chloride solution, this step is conducted under milder, weakly acidic conditions, resulting in improved yield. After the reaction, only a small amount of weak base (e.g., sodium carbonate solution) is required for neutralization. Compared with Method (1), Method (2) reduces base consumption and simplifies post-treatment procedures. Following nitro reduction, the obtained intermediate 6 reacts with various isocyanates (intermediate 7), such as phenyl isocyanate, phenyl isocyanate derivatives, and cycloalkyl isocyanates, to yield the target products. The preparation of isocyanates has been well-documented in prior literature and is not reiterated here.

In an embodiment, the intermediate 6 and the intermediate 7 are reacted in the presence of a catalyst, where the catalyst is triethylamine. A molar ratio of the intermediate 6 to the intermediate 7 is in a range of 1:(3-7). A reaction system further includes dichloromethane as a solvent, and the reaction is conducted for 60 h to 90 h.

In an embodiment, during preparation of intermediate 5, under a condition that the substituted compound has a structure shown in

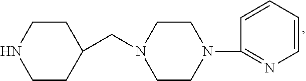 [SMILES: c1ccc(N2CCN(CC3CCNCC3)CC2)nc1]

the substituted compound is prepared by a process including:
subjecting tert-butyl 4-(bromomethyl)piperidin-1-carboxylate, 1-(2-pyridyl)piperazine, K₂CO₃, and the sodium iodide to a reaction under heating to obtain tert-butyl 4-((4-(pyridin-2-yl)piperazin-1-yl)methyl)piperidin-1-carboxylate; and
subjecting the tert-butyl 4-((4-(pyridin-2-yl)piperazin-1-yl)methyl)piperidin-1-carboxylate and concentrated hydrochloric acid to a reaction, and then subjecting a resulting reaction product to basification to a pH value of 10 to obtain the substituted compound.

In an embodiment, the concentrated hydrochloric acid has a mass concentration of 37%.

In an embodiment, during the preparation of the substituted compound, a molar ratio of the tert-butyl 4-(bromomethyl)piperidin-1-carboxylate, the 1-(2-pyridyl)piperazine, the K₂CO₃, and the sodium iodide is in a range of 1:(0.8-1):(0.8-1.2):(0.8-1.2). A reaction system further includes acetonitrile as a solvent, and the reaction is conducted at a temperature of 70° C. to 80° C. for 40 h to 60 h.

In an embodiment, during the preparation of the substituted compound, a molar ratio of the tert-butyl 4-((4-(pyridin-2-yl)piperazin-1-yl)methyl)piperidin-1-carboxylate and the concentrated hydrochloric acid is in a range of 1:(1.6-2.8). A reaction system further includes methanol as a solvent, and the reaction is conducted for 2 h to 6 h.

The synthetic route for the target product in some embodiments of the present disclosure offers the following advantages: (1) Low cost: the starting material 4-chloro-6,7-dimethoxyquinazoline is commercially available at low cost. Alternatively, the target product can be prepared from 6,7-dimethoxyquinazolin-4(3H)-one through a one-step reaction with POCl₃ or SOCl₂ and DMF under heating. (2) Streamlined synthesis: conventional approaches generally employ acyl (e.g., acetyl, butyryl) or benzyl groups to protect the phenolic hydroxyl groups at the 6- or 7-position of the quinazoline ring. After reacting with aniline derivatives, the protecting groups are removed. While the conventional approaches yield quinazoline derivatives modified at the 6- or 7-position, they involve lengthy synthetic routes and result in low overall yields. Acyl protection is only suitable for synthesizing quinazolinyl-anilino derivatives (e.g., Gef). When the quinazoline ring is substituted at the 4-position by strongly basic benzylamine derivatives, the protecting groups (e.g., acetyl) are prone to deprivation under alkaline conditions, preventing successful achievement of the acyl-protected target product. Additionally, acyl-protected 4-chloroquinazolines (e.g., 6-acetoxy-4-chloro-7-methoxyquinazoline) are expensive. (3) Innovative strategy: the method in the present disclosure abandons the conventional “protection-deprotection” approach for phenolic hydroxyl groups. Instead of using acyl or benzyl protecting groups, the method adopts a “selective demethylation at the 6-position of the quinazoline ring” strategy. The method solves the bottleneck problem of efficiently synthesizing quinazolinyl-aryl urea derivatives featuring a benzylamino modification at the 4-position and a piperazinylalkoxy modification at the 6-position on quinazolinyl-aryl urea derivative.

In order to solve the above technical problems, a third object of the present disclosure is to provide a drug, including the piperazinyl-modified quinazolinyl-aryl urea derivative or the pharmaceutically acceptable salt thereof, or a preparation of the piperazinyl-modified quinazolinyl-aryl urea derivative in combination with a suitable auxiliary material.

In an embodiment, the preparation includes a pharmaceutically acceptable excipient and/or a pharmaceutically acceptable additive.

In an embodiment, the preparation is selected from the group consisting of an oral preparation, an injectable preparation, a topical preparation, and an intracavitary administration preparation.

In an embodiment, the oral preparation is a tablet, a capsule, a granule, a solution, a suspension, or an emulsion.

In an embodiment, the injectable preparation is a solution injection, a suspension intravenous injection, an emulsion injection, or a sterile powder for injection.

In an embodiment, administration routes for the injectable preparation include intraperitoneal injection, intravenous injection, subcutaneous injection, intradermal injection, or intratumoral injection.

In an embodiment, the topical preparation is administered via an ointment, a cream, or a patch applied to a skin surface.

In an embodiment, the intracavitary administration preparation includes a bladder instillation preparation, a vaginal preparation, a suppository, or a lotion.

In an embodiment, the excipient is common types available in the pharmaceutical field, including at least one of the following for oral preparation or intracavitary preparation: a binder, a lubricant, a disintegrant, a solubilizer, a diluent, a glidant, a flavoring agent, and a substrate.

In an embodiment, the binder includes at least one of sodium alginate, sodium carboxymethyl cellulose, hydroxyethyl cellulose, hydroxypropyl cellulose, hydroxypropyl methylcellulose, methyl cellulose, gelatin, polyvinylpyrrolidone, cellulose acetate, or starch paste.

In an embodiment, the disintegrant includes at least one of dry starch, sodium carboxymethyl starch, croscarmellose sodium, low-substituted hydroxypropyl cellulose, crospovidone, or effervescent disintegrant.

In an embodiment, the diluent includes at least one of starch, pregelatinized starch, microcrystalline cellulose, dextrin, sucrose, lactose, mannitol, sorbitol, or calcium sulfate dihydrate.

In an embodiment, the additive includes at least one of the following for the injectable preparation: a preservative, an osmotic pressure regulator, and a pH regulator; as well as a substrate for the topical preparation.

In an embodiment, the preservative includes at least one of sodium p-hydroxybenzoate, phenol, benzyl alcohol, chlorobutanol, thimerosal, cresol, or chlorocresol.

In an embodiment, the osmotic pressure regulator includes at least one of sodium chloride, glucose, or glycerol.

In an embodiment, the substrate includes at least one of beeswax, liquid paraffin, petrolatum, solid paraffin, lanolin, cetaceum, stearic acid, PEG4000, carbomer, or sodium carboxymethyl cellulose.

In an embodiment, the drug is administered orally or parenterally (e.g., intravenously, subcutaneously, intraperitoneally, intracavitarily, or topically). For drugs unstable under gastric conditions, they may be formulated as an enteric-coated tablet, an enteric-coated capsule, a sustained-release tablet, or a sustained-release capsule.

In order to solve the above technical problems, a fourth object of the present disclosure is to provide use of the piperazinyl-modified quinazolinyl-aryl urea derivative or the pharmaceutically acceptable salt thereof in manufacture of a medicament for treating a tumor, where the tumor is at least one selected from the group consisting of bladder cancer, osteosarcoma, pancreatic cancer, breast cancer, cervical cancer, NSCLC (non-small cell lung cancer), ovarian cancer, hepatoblastoma, liver cancer, prostate cancer, renal carcinoma, and colorectal cancer.

Compared with the prior art, some embodiments of the present disclosure have the following beneficial effects:

1. In the compounds of the present disclosure, the 4-aminoquinazolinyl group and the carbamido group are two critical pharmacophores, particularly the carbamido group. Removal of the carbamido group leads to a sharp decline in activity. While replacing the quinazoline ring with other aromatic rings (e.g., pyridine, pyrazine) retains some activity, efficacy is reduced, indicating that the quinazoline ring is the optimal aromatic moiety on the left side of the molecule. The phenyl group on the far right should not be replaced by alkyl groups, as this causes a drastic loss or complete elimination of activity. However, substitution with benzyl or cycloalkyl groups maintains potent antiproliferative activity against certain cell lines. Substituents on the terminal benzene ring significantly impact activity: replacing hydrogen with ethyl, tert-butyl, chlorine atom, or trifluoromethyl markedly enhances activity of benzene ring, especially with strong hydrophobic groups like 2,6-diethyl or 4-tert-butyl. Trifluoromethyl and chlorine atom substitutions offer moderate improvements. The alkoxy chain at the 6-position of the quinazoline ring modulates lipid-water partition coefficient, thereby influencing absorption, distribution, and metabolism in vivo; chain lengths of 3 to 7 carbon atoms are optimal. Substitution with methoxy, ethoxy, or propoxy groups at the 6-position hydrogen of quinazoline ring results in insufficient receptor selectivity and suboptimal antitumor efficacy. These derivatives also exhibit poor lipid/water solubility, limiting formulation to oral delivery and hindering development of injectable or intracavitary perfusion preparations. 2. SciFinder® searches confirm that all piperazinyl-modified quinazolinyl-aryl urea derivatives disclosed herein are novel compounds. Experimental results show sensitivity of four human bladder cancer cell lines (J82, UM-UC-3, T24, 5637) to compounds 8-1j, 8-2h, and 8-2j. Two osteosarcoma cell lines (143B, MG-63) are sensitive to 8-1j, 8-2h, and 8-2z. Pancreatic cancer cell line BxPC-3 is highly sensitive to 8-2h but less sensitive to 8-1j. Breast cancer cell line MCF-7 is sensitive to 8-1j and 8-1r, while cervical cancer cell line HeLa is sensitive to 8-2h and 8-2j. Representative compound 8-1j modulates the FoxM1/KIF20A/Nrf2/IRP2 signaling pathway and arrests the cell cycle at the G2/M phase. Compound 8-1j demonstrates potent antitumor activity in two tumor-bearing nude mouse models, and 8-2h shows significant efficacy in a human pancreatic cancer tumor-bearing nude mouse model, highlighting their potential as molecularly targeted antitumor agents. Furthermore, several target compounds in the present disclosure exhibit excellent solubility in diverse solvents (e.g., PEG400+normal saline, PEG400+PBS, and PEG400+5% glucose injection), supporting multiple administration routes including oral, injectable, and intravesical instillation.

### BRIEF DESCRIPTION OF THE DRAWINGS

FIG. 1 shows the synthetic routes for compounds 8-1a to 8-1v and 8-2a to 8-2z in the examples of the present disclosure;

FIG. 2 shows the synthetic route for intermediate 5-2 in Example 15 of the present disclosure;

FIG. 3 shows the ¹H NMR spectrum of intermediate 2 in an embodiment of the present disclosure;

FIG. 4 shows the ¹H NMR spectrum of intermediate 3 in an embodiment of the present disclosure;

FIG. 5 shows the ¹H NMR spectrum of intermediate 4 in an embodiment of the present disclosure;

FIG. 6 shows the ¹H NMR spectrum of intermediate 5-1 in an embodiment of the present disclosure;

FIG. 7 shows the ¹H NMR spectrum of compound 8-1j in an embodiment of the present disclosure;

FIG. 8 shows the ¹H NMR spectrum of compound 8-1r in an embodiment of the present disclosure;

FIG. 9 shows the ¹H NMR spectrum of intermediate 10 in an embodiment of the present disclosure;

FIG. 10 shows the ¹H NMR spectrum of intermediate 11 in an embodiment of the present disclosure;

FIG. 11 shows the ¹H NMR spectrum of intermediate 5-2 in an embodiment of the present disclosure;

FIG. 12 shows the ¹H NMR spectrum of intermediate 6-2 in an embodiment of the present disclosure;

FIG. 13 shows the ¹H NMR spectrum of compound 8-2d in an embodiment of the present disclosure;

FIG. 14 shows the ¹H NMR spectrum of compound 8-2e in an embodiment of the present disclosure;

FIG. 15 shows the ¹H NMR spectrum of compound 8-2h in an embodiment of the present disclosure;

FIG. 16 shows the ¹H NMR spectrum of compound 8-2n in an embodiment of the present disclosure;

FIG. 17 shows the ¹H NMR spectrum of compound 8-2t in an embodiment of the present disclosure;

FIG. 18 shows the ¹H NMR spectrum of compound 8-2v in an embodiment of the present disclosure;

FIG. 19 shows the ¹H NMR spectrum of compound 8-2z in an embodiment of the present disclosure;

FIG. 20 shows the ¹³C NMR spectrum of compound 8-1c in an embodiment of the present disclosure;

FIG. 21 shows the ¹³C NMR spectrum of compound 8-1d in an embodiment of the present disclosure;

FIG. 22 shows the ¹³C NMR spectrum of compound 8-1h in an embodiment of the present disclosure;

FIG. 23 shows the ¹³C NMR spectrum of compound 8-1j in an embodiment of the present disclosure;

FIG. 24 shows the ¹³C NMR spectrum of compound 8-1o in an embodiment of the present disclosure;

FIG. 25 shows the ¹³C NMR spectrum of compound 8-1r in an embodiment of the present disclosure;

FIG. 26 shows the ¹³C NMR spectrum of compound 8-2b in an embodiment of the present disclosure;

FIG. 27 shows the ¹³C NMR spectrum of compound 8-2c in an embodiment of the present disclosure;

FIG. 28 shows the ¹³C NMR spectrum of compound 8-2h in an embodiment of the present disclosure;

FIG. 29 shows the ¹³C NMR spectrum of compound 8-2j in an embodiment of the present disclosure;

FIG. 30 shows the ¹³C NMR spectrum of compound 8-2z in an embodiment of the present disclosure;

FIG. 31 shows the HBMC spectrum of intermediate 3 in an embodiment of the present disclosure;

FIG. 32 shows the HSQC spectrum of intermediate 3 in an embodiment of the present disclosure;

FIG. 33 shows the HRMS spectrum of compound 8-1j in an embodiment of the present disclosure;

FIG. 34 shows the HRMS spectrum of compound 8-2a in an embodiment of the present disclosure;

FIG. 35 shows the HRMS spectrum of compound 8-2h in an embodiment of the present disclosure;

FIG. 36 shows the HRMS spectrum of compound 8-2j in an embodiment of the present disclosure;

FIG. 37 shows the HRMS spectrum of compound 8-2r in an embodiment of the present disclosure;

FIG. 38 shows the HRMS spectrum of compound 8-2s in an embodiment of the present disclosure;

FIG. 39 shows the HRMS spectrum of compound 8-2u in an embodiment of the present disclosure;

FIG. 40 shows the HRMS spectrum of compound 8-2w in an embodiment of the present disclosure;

FIG. 41 shows the HRMS spectrum of compound 8-2y in an embodiment of the present disclosure;

FIG. 42A shows the proliferation inhibition activity chart of several typical compounds in some embodiments after co-incubation with J82 cells for different durations, FIG. 42B shows the proliferation inhibition activity chart of several typical compounds in some embodiments after co-incubation with UM-UC-3 cells for different durations, FIG. 42C shows the proliferation inhibition activity chart of several typical compounds in some embodiments after co-incubation with T24 cells for different durations, and FIG. 42D shows the proliferation inhibition activity chart of several typical compounds in some embodiments after co-incubation with 5637 cells for different durations (NOTE: Sora: sorafenib; Erda: erdafitinib);

FIG. 43A shows the proliferation inhibition activity chart of several typical compounds in some embodiments after co-incubation with 143B cells for different durations, FIG. 43B shows the proliferation inhibition activity chart of several typical compounds in some embodiments after co-incubation with MG-63 cells for different durations, FIG. 43C shows the proliferation inhibition activity chart of several typical compounds in some embodiments after co-incubation with BxPC-3 cells for different durations, FIG. 43D shows the proliferation inhibition activity chart of several typical compounds in some embodiments after co-incubation with PANC-1 cells for different durations, FIG. 43E shows the proliferation inhibition activity chart of several typical compounds in some embodiments after co-incubation with HeLa cells for different durations, FIG. 43F shows the proliferation inhibition activity chart of several typical compounds in some embodiments after co-incubation with MCF-7 cells for different durations, FIG. 43G shows the proliferation inhibition activity chart of several typical compounds in some embodiments after co-incubation with SV-HUC-1 cells for different durations, and FIG. 43H shows the proliferation inhibition activity chart of several typical compounds in some embodiments after co-incubation with WI-8 cells for different durations (NOTE: A-2 is 9g; Sora: sorafenib; Reg: regorafenib; Gem: gemcitabine; Apat: apatinib mesylate; PTX: paclitaxel; CDDP: cisplatin);

FIG. 44A shows the arrest of T24 cell cycle induced by compound 8-1j in an embodiment, and FIG. 44B shows G2/M phase cell ratio changes before and after the treatment of compound 8-1j in an embodiment;

FIG. 45A shows the arrest of UM-UC-3 cell cycle induced by compound 8-2j in an embodiment, and FIG. 45B shows G2/M phase cell ratio changes before and after the treatment of compound 8-2j in an embodiment;

FIG. 46A shows the arrest of 143B cell cycle induced by compound 8-1j in an embodiment, and FIG. 46B shows G2/M phase cell ratio changes before and after the treatment of compound 8-1j in an embodiment;

FIG. 47A shows the arrest of BxPC-3 cell cycle induced by compound 8-2h in an embodiment that, and FIG. 47B shows cell ratio changes in each stage before and after the treatment of 8-2h in an embodiment;

FIG. 48A shows the regulation of cell cycle- and ferroptosis-related proteins by compound 8-1j in an embodiment, FIG. 48B shows relative expression levels changes of FOXM1 and KIF20A in an embodiment, FIG. 48C shows relative expression levels changes of Nrf2 and FTH1 in an embodiment, FIG. 48D shows relative expression levels changes of xCT and GPx4 in an embodiment, and FIG. 48E shows relative expression levels changes of HO-1 and IRP2 in an embodiment;

FIG. 49 shows the potential mechanism of action of compound 8-1j in an embodiment;

FIG. 50A shows the photograph of the J82 tumor-bearing nude mouse model in an embodiment, FIG. 50B shows the photograph of the excised tumors in J82 tumor-bearing nude mouse model in an embodiment, FIG. 50C shows the inhibition of tumor volume by compound 8-1j in an embodiment in the J82 tumor-bearing nude mouse model, FIG. 50D shows the changes in body weight induced by compound 8-1j in an embodiment in the J82 tumor-bearing nude mouse model, and FIG. 50E shows the inhibition of tumor weight by compound 8-1j in an embodiment in the J82 tumor-bearing nude mouse model (NOTE: A-2 in the figure is 9g);

FIG. 51A shows the photograph of the 143B tumor-bearing nude mouse model in an embodiment, FIG. 51B shows the photograph of the excised tumors in 143B tumor-bearing nude mouse model in an embodiment, FIG. 51C shows the inhibition of tumor volume by compound 8-1j in an embodiment in the 143B tumor-bearing nude mouse model, FIG. 51D shows the changes in body weight induced by compound 8-1j in an embodiment in the 143B tumor-bearing nude mouse model, and FIG. 51E shows the inhibition of tumor weight by compound 8-1j in an embodiment in the 143B tumor-bearing nude mouse model; and

FIG. 52A shows the photograph of the BxPC-3 tumor-bearing nude mouse model in an embodiment, FIG. 52B shows the photograph of the excised tumors in BxPC-3 tumor-bearing nude mouse model in an embodiment, FIG. 52C shows the inhibition of tumor volume by compound 8-2h in an embodiment in the BxPC-3 tumor-bearing nude mouse model, FIG. 52D shows the changes of body weight induced by compound 8-2h in an embodiment in the BxPC-3 tumor-bearing nude mouse model, and FIG. 52E shows the inhibition of tumor weight by compound 8-2h in an embodiment in the BxPC-3 tumor-bearing nude mouse model.

### DETAILED DESCRIPTION OF THE EMBODIMENTS

The technical solutions in the embodiments of the present application are clearly and completely described below with reference to the drawings in the embodiments of the present application. Apparently, the described embodiments are merely some rather than all of the embodiments of the present application. All other embodiments obtained by those of ordinary skill in the art based on the embodiments of the present disclosure without inventive efforts should fall within the scope of the present disclosure.

In order to further illustrate the present disclosure, the technical solutions provided by the present disclosure are described in detail below in connection with accompanying drawings and examples, but these examples should not be understood as limiting the claimed scope of the present disclosure. The reagents and drug raw materials used in the following embodiments of the present disclosure are, unless otherwise specified, obtained commercially, and the same raw materials are used in parallel experiments.

### Examples 1 to 37

Series I. Morpholinoethylpiperazinylhexyl-modified quinazolinyl-aryl urea derivatives, including compounds 8-1a to 8-1v had structures corresponding to those in Examples 1 to 14, with chemical structural formulas and product codes shown in Table 1.

TABLE 1
Chemical structural formulas and product codes of morpholinoethylpiperazinylhexyl-
modified quinazolinyl-aryl urea derivatives

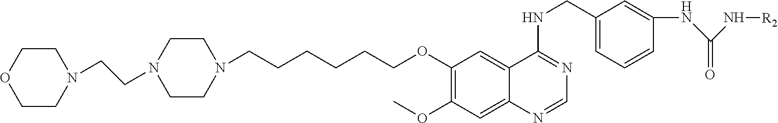

| Example | R2 | Product code | Example | R2 | Product code |
|---|---|---|---|---|---|
| 1 | 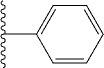 [SMILES: *c1ccccc1] | Compound 8-1a | 8 | 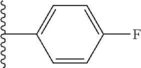 [SMILES: *c1ccc(F)cc1] | Compound 8-11 |
| 2 | 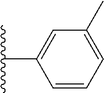 [SMILES: *c1cccc(C)c1] | Compound 8-1c | 9 | 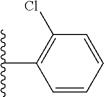 [SMILES: *c1ccccc1Cl] | Compound 8-1m |
| 3 | 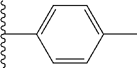 [SMILES: *c1ccc(C)cc1] | Compound 8-1d | 10 | 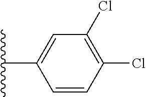 [SMILES: *c1ccc(Cl)c(Cl)c1] | Compound 8-10 |
| 4 | 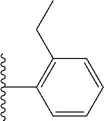 [SMILES: *c1ccccc1CC] | Compound 8-1g | 11 | 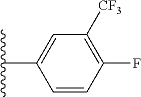 [SMILES: *c1ccc(F)c(C(F)(F)F)c1] | Compound 8-1q |
| 5 | 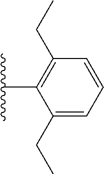 [SMILES: *c1c(CC)cccc1CC] | Compound 8-1h | 12 | 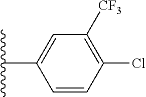 [SMILES: *c1ccc(Cl)c(C(F)(F)F)c1] | Compound 8-1r |
| 6 | 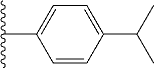 [SMILES: *c1ccc(C(C)C)cc1] | Compound 8-1i | 13 | 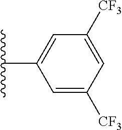 [SMILES: *c1cc(C(F)(F)F)cc(C(F)(F)F)c1] | Compound 8-1s |
| 7 | 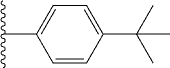 [SMILES: *c1ccc(C(C)(C)C)cc1] | Compound 8-1j | 14 | 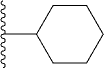 [SMILES: *C1CCCCC1] | Compound 8-1v |

Series II: Pyriypiperazinylmethypiperidyhexyl-modified quinazolinyl-aryl urea derivatives, including compounds 8-2a to 8-2z had structures corresponding to those in Examples 15 to 37, with chemical structural formulas and product codes shown in Table 2.

TABLE 2
Chemical structural formulas and product codes of
pyridylpiperazinylmethylpiperidinylhexyl-modified quinazolinyl-aryl urea derivatives

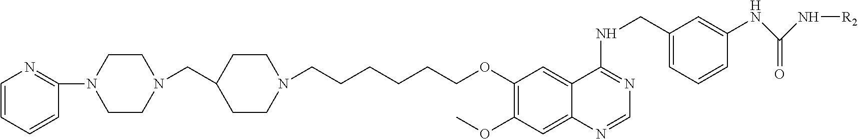

| Example | R | Product code | Example | R | Product code |
|---|---|---|---|---|---|
| 15 | 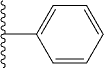 [SMILES: *c1ccccc1] | Compound 8-2a | 27 | 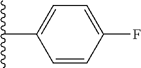 [SMILES: *c1ccc(F)cc1] | Compound 8-2n |
| 16 | 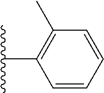 [SMILES: *c1ccccc1C] | Compound 8-2b | 28 | 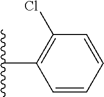 [SMILES: *c1ccccc1Cl] | Compound 8-20 |
| 17 | 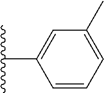 [SMILES: *c1cccc(C)c1] | Compound 8-2c | 29 | 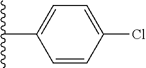 [SMILES: *c1ccc(Cl)cc1] | Compound 8-2q |
| 18 | 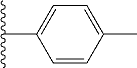 [SMILES: *c1ccc(C)cc1] | Compound 8-2d | 30 | 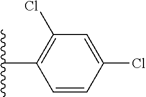 [SMILES: *c1ccc(Cl)cc1Cl] | Compound 8-2r |
| 19 | 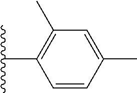 [SMILES: *c1ccc(C)cc1C] | Compound 8-2e | 31 | 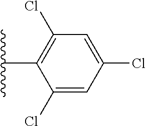 [SMILES: *c1c(Cl)cc(Cl)cc1Cl] | Compound 8-2s |
| 20 | 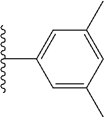 [SMILES: *c1cc(C)cc(C)c1] | Compound 8-2f | 32 | 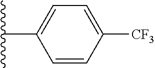 [SMILES: *c1ccc(C(F)(F)F)cc1] | Compound 8-2t |
| 21 | 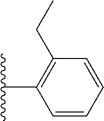 [SMILES: *c1ccccc1CC] | Compound 8-2g | 33 | 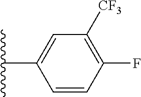 [SMILES: *c1ccc(F)c(C(F)(F)F)c1] | Compound 8-2u |
| 22 | 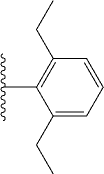 [SMILES: *c1c(CC)cccc1CC] | Compound 8-2h | 34 | 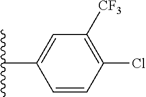 [SMILES: *c1ccc(Cl)c(C(F)(F)F)c1] | Compound 8-2v |
| 23 | 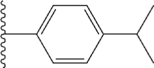 [SMILES: *c1ccc(C(C)C)cc1] | Compound 8-2i | 35 | 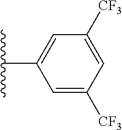 [SMILES: *c1cc(C(F)(F)F)cc(C(F)(F)F)c1] | Compound 8-2w |
| 24 | 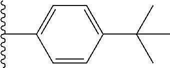 [SMILES: *c1ccc(C(C)(C)C)cc1] | Compound 8-2j | 36 | 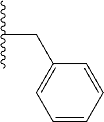 [SMILES: *Cc1ccccc1] | Compound 8-2y |
| 25 | 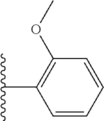 [SMILES: *c1ccccc1OC] | Compound 8-2k | 37 | 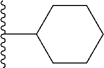 [SMILES: *C1CCCCC1] | Compound 8-2z |
| 26 | 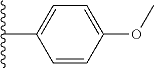 [SMILES: *c1ccc(OC)cc1] | Compound 8-21 | | | |

### Example 1

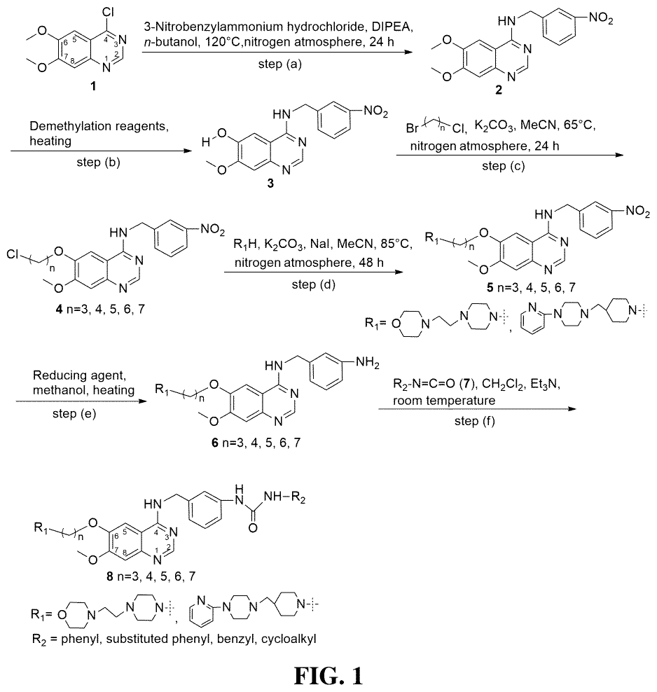

### N-(3-(((7-methoxy-6-((6-(4-(2-morpholinoethyl)piperazin-1-yl)hexyl)oxy)quinazolin-4-yl)a-mino)methyl)phenyl)-N′-phenylurea (compound 8-1a) was prepared by procedures as illustrated in FIG. 1

Step (a): synthesis of 6,7-dimethoxy-N-(3-nitrobenzyl)quinazolin-4-amine (intermediate 2), structural formula:

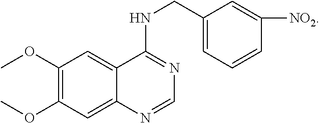 [SMILES: COc1cc2ncnc(NCc3cccc([N+](=O)[O-])c3)c2cc1OC]

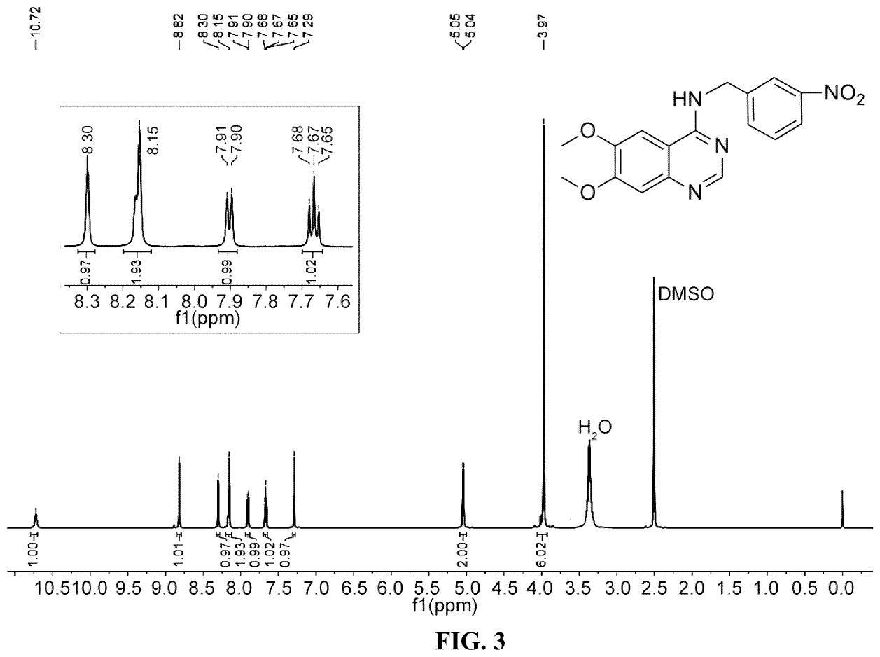

Under a nitrogen atmosphere, 4-chloro-6,7-dimethoxyquinazoline (compound 1, 28.58 g, 127 mmol), 3-nitrobenzylamine hydrochloride (20.0 g, 106 mmol), N,N-diisopropylethylamine (DIPEA, 20 mL, 114.8 mmol), and n-butanol (120 mL) were added sequentially to a 250 mL round-bottom flask. A resulting mixture was stirred until dissolved, then gradually heated to 120° C. under reflux and stirred for 24 h to conduct a reaction. After completion of the reaction, a resulting product was slightly cooled down, and a solvent was removed under reduced pressure upon cooling. Upon cooling to room temperature, a yellow solid precipitated. The yellow solid was washed 3 times with petroleum ether via suction filtration. A resulting solid was recrystallized from ethyl acetate, and then dried, yielding the yellow solid (intermediate 2) in 75% yield. The ¹H NMR spectrum is shown in FIG. 3. ¹H NMR (600 MHz, DMSO-d₆) δ 10.72 (s, 1H), 8.82 (s, 1H), 8.30 (s, 1H), 8.15 (s, 2H), 7.90 (d, J=7.6 Hz, 1H), 7.67 (t, J=7.9 Hz, 1H), 7.29 (s, 1H), 5.04 (d, J=5.7 Hz, 2H), 3.97 (s, 6H). ¹³C NMR (150 MHz, DMSO-d₆) δ 159.76, 156.43, 150.41, 149.67, 148.30, 140.69, 134.84, 130.56, 122.79, 122.74, 107.25, 104.23, 100.29, 57.22, 56.86, 44.22. HRMS (ESI, m z): calcd. for C₁₇H₁₆N₄O₄ [M+H]⁺: 341.1250; found: 341.1245.

Step (b): synthesis of 7-methoxy-4-((3-nitrobenzyl)amino)quinazolin-6-ol (intermediate 3), structural formula:

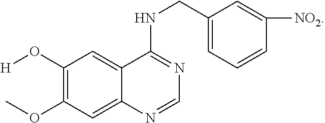 [SMILES: COc1cc2ncnc(NCc3cccc([N+](=O)[O-])c3)c2cc1O]

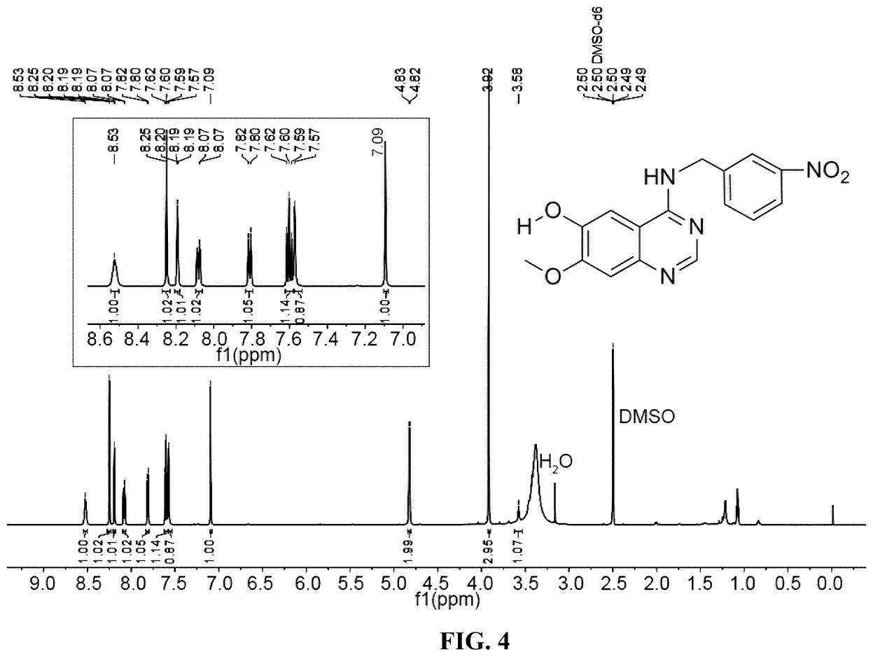

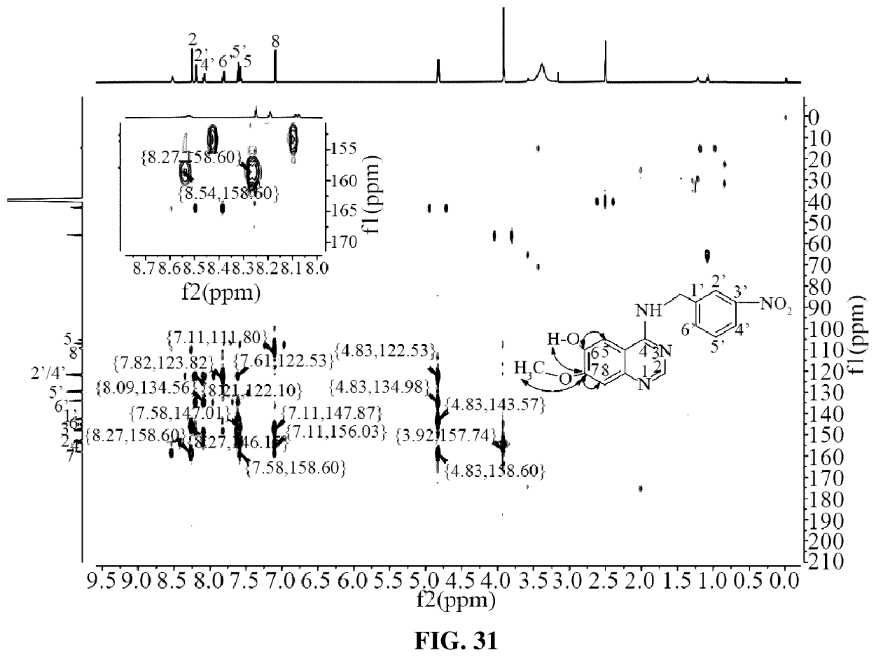

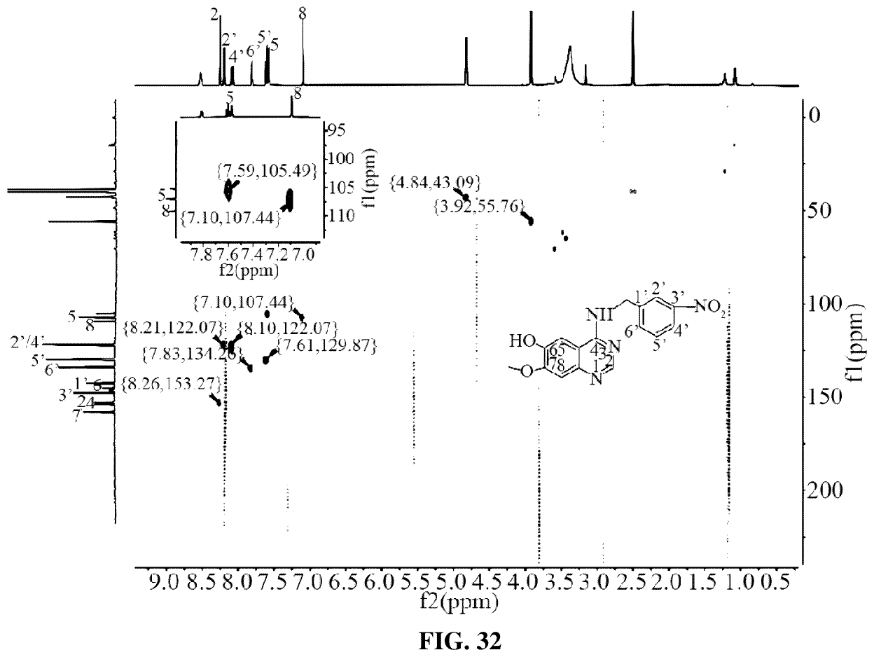

Dried intermediate 2 (10.0 g, 29.38 mmol), L-methionine (6.58 g, 44.07 mmol), and concentrated sulfuric acid (98 wt %, 52.88 g, 528.84 mmol as H₂SO₄) were added sequentially to a 250 mL round-bottom flask. After complete dissolution, the flask was placed in an oil bath under nitrogen protection and gradually heated to 100° C. with stirring for 12 h. Upon reaction completion, a resulting mixture was cooled to room temperature. A resulting solution was poured slowly into an ice-water mixture (100 mL) with stirring. After standing, a resulting suspension was centrifuged to collect a solid precipitate. An aqueous NaOH solution was added to the solid precipitate in batches to adjust the pH value to 7-8. The solid precipitate was dried to obtain a tan crude product, which was purified by column chromatography to yield intermediate 3 as a pale yellow solid (6.90 g, 72% yield). The ¹H NMR spectrum is shown in FIG. 4, the HMBC spectrum is shown in FIG. 31, and the HSQC spectrum is shown in FIG. 32. ¹H NMR (600 MHz, DMSO-d6) δ 8.53 (t, J=6.0 Hz, 1H), 8.25 (s, 1H), 8.19 (t, J=2.0 Hz, 1H), 8.07 (d, J=1.4 Hz, 1H), 7.81 (d, J=7.8 Hz, 1H), 7.60 (t, J=7.9 Hz, 1H), 7.57 (s, 1H), 7.09 (s, 1H), 4.82 (d, J=5.9 Hz, 2H), 3.92 (s, 3H), 3.58 (s, 1H). ¹³C NMR (150 MHz, DMSO-d6) δ 157.88, 153.67, 152.66, 147.80, 146.52, 145.21, 142.66, 134.07, 129.77, 121.70, 121.67, 109.14, 107.04, 105.24, 55.77, 42.93. HRMS (ESI, m z): calcd. for C₁₆H₁₄N₄O₄ [M+H]⁺: 327.1093; found: 327.1083.

Step (c): synthesis of 6-((6-chlorohexyl)oxy)-7-methoxy-N-(3-nitrobenzyl)quinazolin-4-amine (n=6, intermediate 4), structural formula:

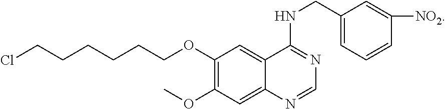 [SMILES: COc1cc2ncnc(NCc3cccc([N+](=O)[O-])c3)c2cc1OCCCCCCCl]

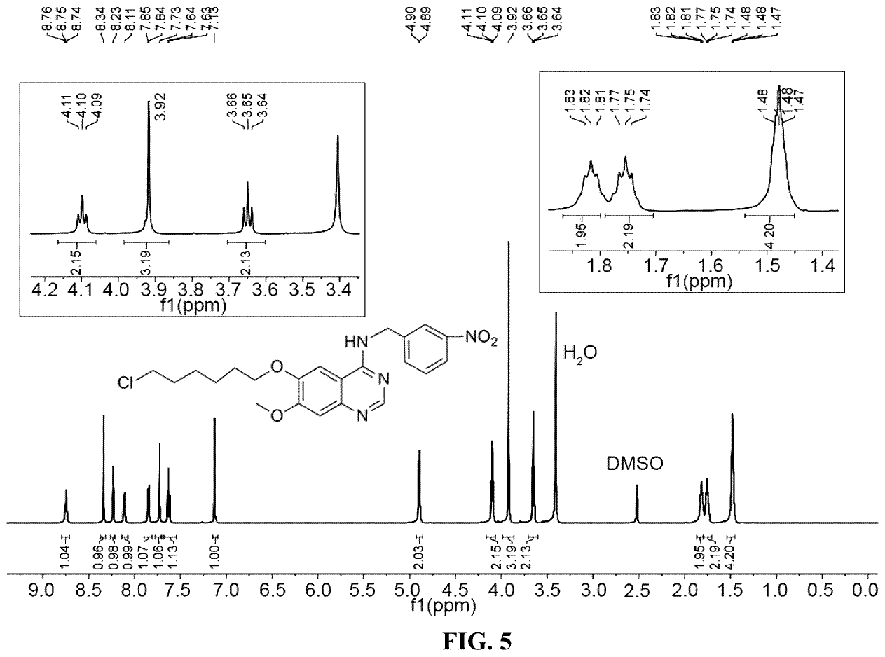

7-Methoxy-4-((3-nitrobenzyl)amino)quinazolin-6-ol (intermediate 3, 5.0 g, 15.32 mmol), 1-bromo-6-chlorohexane (3.43 mL, 22.98 mmol), K₂CO₃ (2.12 g, 15.32 mmol), and acetonitrile (50 mL) were added sequentially into a flask. Under nitrogen protection, a resulting mixture was gradually heated to 65° C. and stirred for 24 h. Upon completion, insoluble materials were removed by filtration. A resulting filtrate was collected, concentrated and then purified by silica gel column chromatography to obtain intermediate 4 as a pale yellow solid (85% yield). The ¹H NMR spectrum is shown in FIG. 5. ¹H NMR (600 MHz, DMSO-d₆) δ 8.75 (t, J=5.8 Hz, 1H), 8.34 (s, 1H), 8.23 (s, 1H), 8.11 (dd, J=8.1, 1.5 Hz, 1H), 7.85 (d, J=7.7 Hz, 1H), 7.73 (s, 1H), 7.63 (t, J=7.9 Hz, 1H), 7.13 (s, 1H), 4.89 (d, J=5.8 Hz, 2H), 4.10 (t, J=6.5 Hz, 2H), 3.92 (s, 3H), 3.65 (t, J=6.6 Hz, 2H), 1.82 (t, J=1.2 Hz, 2H), 1.75 (t, J=1.2 Hz, 2H), 1.53-1.41 (m, 4H). ¹³C NMR (150 MHz, DMSO-d₆) δ 158.57, 154.48, 153.80, 148.29, 148.23, 146.47, 142.96, 134.50, 130.26, 122.21, 122.19, 108.93, 107.54, 103.07, 68.98, 56.18, 45.82, 43.35, 32.46, 28.95, 26.56, 25.38. HRMS (ESI, m/z): calcd. for C₂₂H₂₅ClN₄O₄ [M+H]⁺: 445.1643; found: 445.1623.

Step (d): synthesis of 7-methoxy-6-((6-(4-(2-morpholinoethyl)piperazin-1-yl)hexyl)oxy)-N-(3-nitrobenzyl)quinazolin-4-amine (intermediate 5-1), structural formula:

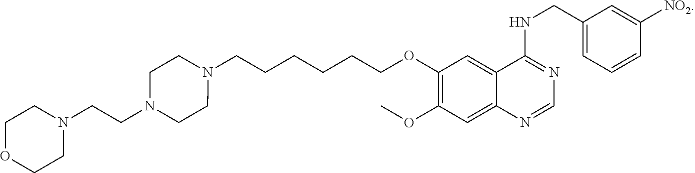 [SMILES: COc1cc2ncnc(NCc3cccc([N+](=O)[O-])c3)c2cc1OCCCCCCN1CCN(CCN2CCOCC2)CC1]

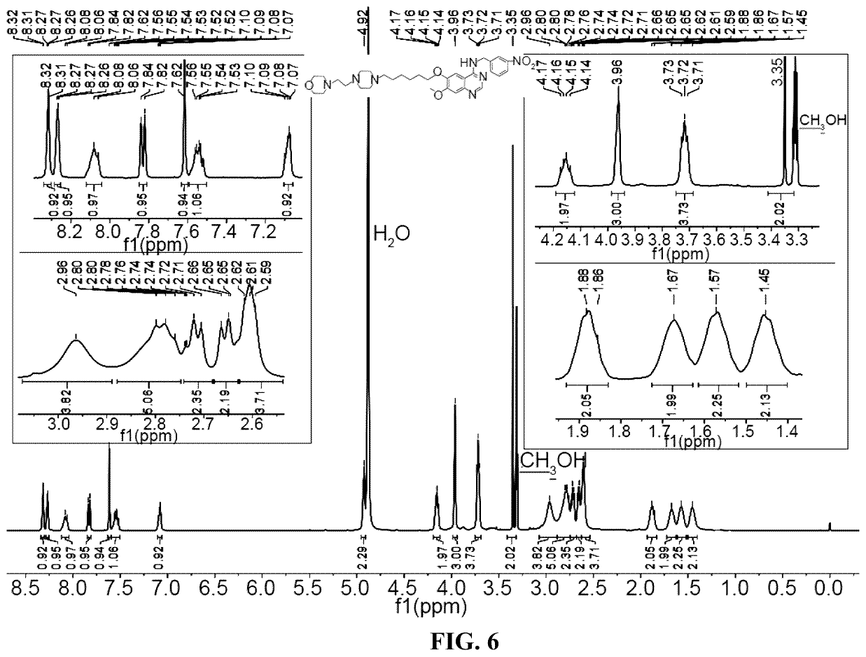

In a dried round-bottom flask, the intermediate 4 (5.0 g, 11.31 mmol), 4-(2-(piperazin-1-yl)ethyl)morpholine (2.48 g, 12.44 mmol), anhydrous K₂CO₃ (1.56 g, 11.31 mmol), NaI (1.86 g, 12.44 mmol), and acetonitrile (100 mL) were added sequentially. A resulting mixture was stirred at room temperature under nitrogen atmosphere for 10 min, then gradually heated to 85° C. under reflux and stirred for 48 h. After cooling to room temperature, insoluble solids were removed by filtration. A resulting filtrate was concentrated under reduced pressure and purified by column chromatography to obtain intermediate 5-1 as a pale yellow solid (6.19 g, 90% yield). The ¹H NMR spectrum is shown in FIG. 6. m.p. 118.2-119.6° C. ¹H NMR (400 MHz, CD₃OD) δ 8.32 (d, J=2.0 Hz, 1H), 8.27 (t, J=2.2 Hz, 1H), 8.07 (d, J=8.8 Hz, 1H), 7.83 (d, J=7.7 Hz, 1H), 7.62 (s, 1H), 7.54 (dt, J=9.9, 5.4 Hz, 1H), 7.12-7.05 (m, 1H), 4.92 (s, 2H), 4.16 (q, J=5.9, 5.1 Hz, 2H), 3.96 (s, 3H), 3.72 (t, J=4.7 Hz, 4H), 3.35 (s, 2H), 3.07-2.89 (m, 4H), 2.87-2.75 (m, 5H), 2.71 (d, J=6.2 Hz, 2H), 2.68-2.63 (m, 2H), 2.61 (d, J=4.5 Hz, 4H), 1.93-1.83 (m, 2H), 1.73-1.63 (m, 2H), 1.62-1.52 (m, 2H), 1.51-1.40 (m, 2H). ¹³C NMR (100 MHz, CD₃OD) δ 160.28, 156.73, 154.37, 150.50, 149.82, 146.44, 143.38, 135.28, 130.84, 123.53, 123.12, 110.23, 107.00, 103.84, 70.60, 67.51, 58.74, 56.75, 56.28, 55.05, 54.98, 53.18, 52.68, 44.83, 30.06, 27.91, 26.99, 26.32. HRMS (m z): calcd. for C₃₂H₄₅N₇O₅ [M+H]⁺: 608.3560; found: 608.3535.

### Step (e): synthesis of N-(3-aminobenzyl)-7-methoxy-6-((6-(4-(2-morpholinoethyl)piperazin-1-yl)hexyl)oxy)quinazolin-4-amine (intermediate 6-1), structural formula

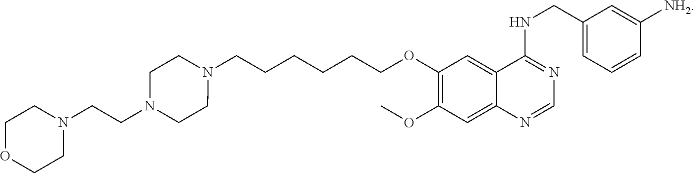 [SMILES: COc1cc2ncnc(NCc3cccc(N)c3)c2cc1OCCCCCCN1CCN(CCN2CCOCC2)CC1]

Preparation of 10 wt % aqueous ammonium chloride solution: ammonium chloride (0.56 g, 10.5 mmol) was dissolved in deionized water (5.04 g) to obtain a 10 wt % aqueous ammonium chloride solution. Activation of iron powder: the iron powder was treated with a 15 wt % aqueous hydrochloric acid solution under stirring. After slowly decanting the supernatant, a resulting solid was collected as an activated iron powder (2.74 g, 49 mmol). Reaction procedure: intermediate 5-1 (4.25 g, 7.0 mmol) and methanol (95 mL) were added into a round-bottom flask and stirred until dissolved. The activated iron powder and an aqueous ammonium chloride solution were added thereto, followed by stirring for 5 min. A resulting mixture was heated to 60° C. and stirred for 12 h. After cooling to room temperature, insoluble materials were removed by filtration. A resulting residue was washed repeatedly with methanol. A resulting combined filtrate was concentrated, then adjusted to pH=7-8 with an aqueous sodium carbonate solution. Extraction was conducted with ethyl acetate 3 times. Solvent was removed after organic layers were merged to obtain a crude product. The crude product was purified by column chromatography to obtain intermediate 6-1 as a pale yellow solid (3.32 g, 82% yield). m.p. 79.2-80.6° C. ¹H NMR (400 MHz, CD₃OD) δ 8.39 (s, 1H), 7.68 (s, 1H), 7.13 (s, 1H), 7.05 (t, J=7.8 Hz, 1H), 6.78 (d, J=2.1 Hz, 1H), 6.72 (d, J=7.6 Hz, 1H), 6.62 (dd, J=7.8, 2.2 Hz, 1H), 4.78 (s, 2H), 4.15 (t, J=6.3 Hz, 2H), 3.98 (s, 3H), 3.83 (t, J=4.7 Hz, 4H), 2.83-2.74 (m, 4H), 2.72-2.64 (m, 4H), 2.63-2.42 (m, 8H), 2.31 (t, J=7.5 Hz, 2H), 1.93-1.85 (m, 2H), 1.79-1.72 (m, 2H), 1.53-1.45 (m, 4H). ¹³C NMR (100 MHz, CD₃OD) δ 159.12, 156.16, 149.64, 147.69, 138.80, 137.59, 128.98, 117.06, 114.27, 114.18, 107.28, 103.07, 101.12, 69.27, 63.86, 56.52, 55.72, 53.28, 52.25, 51.44, 50.96, 49.50, 44.66, 28.39, 25.99, 25.28, 23.65. HRMS (m z): calcd. for C₃₂H₄₇N₇O₃ [M+H]⁺: 578.3819; found: 578.3817.

Step (f): synthesis of compound 8-1a: dried intermediate 6-1 (202 mg, 0.35 mmol), DCM (dichloromethane, 20 mL), phenyl isocyanate (1.40 mmol), and triethylamine (TEA, 0.5 mL) were added into a round-bottom flask. A resulting mixture was stirred at room temperature for 72 h. Upon completion, insoluble materials were removed by filtration. A resulting filtrate was concentrated under reduced pressure and purified by column chromatography to obtain compound 8-1a as a white solid (139 mg, 57% yield). m.p. 104.3-105.9° C. ¹H NMR (400 MHz, CD₃OD) δ 8.44 (s, 1H), 8.22 (s, 1H), 7.51 (s, 1H), 7.40 (s, 1H), 7.32 (d, J=7.9 Hz, 3H), 7.15 (dt, J=8.0, 3.9 Hz, 3H), 7.00 (s, 2H), 6.63 (t, J=5.4 Hz, 1H), 4.72 (d, J=5.3 Hz, 2H), 3.94 (s, 2H), 3.88 (s, 3H), 3.76 (d, J=4.9 Hz, 4H), 2.79-2.46 (m, 10H), 2.44-2.36 (m, 6H), 2.27 (t, J=5.2 Hz, 2H), 1.81-1.74 (m, 2H), 1.71-1.63 (m, 2H), 1.52-1.41 (m, 2H), 1.41-1.32 (m, 2H). ¹³C NMR (100 MHz, CD₃OD) δ 158.98, 154.62, 153.09, 152.74, 148.41, 140.43, 140.39, 139.98, 138.28, 129.18, 128.98, 122.81, 121.06, 118.94, 117.23, 116.77, 114.91, 108.45, 105.26, 69.34, 64.86, 56.16, 55.83, 53.44, 52.68, 51.93, 51.02, 49.87, 44.92, 28.98, 26.52, 25.67, 21.88. HRMS (m z): calcd. for C₃₉H₅₂N₈O₄ [M+H]⁺: 697.4190; found: 697.4171.

According to the above procedures, appropriate starting materials and reagents were selected. By replacing phenyl isocyanate in Step (f) of Example 1 with intermediate 7-1 with a general structural formula of R₂—N═C═O, where R₂ in intermediate 7-1 corresponds to R₂ as defined in Table 1, the morpholinoethylpiperazinylhexyl-modified quinazolinyl-aryl urea derivatives exemplified in Examples 2 to 14 were prepared, respectively. Yields referred specifically to the yield of Step (f). Characterization data for Examples 2 to 14 are listed below.

### Example 2

### N-(3-(((7-methoxy-6-((6-(4-(2-morpholinoethyl)piperazin-1-yl)hexyl)oxy)quinazolin-4-yl)amino)methyl)phenyl)-N′-(3-methylphenyl)urea (compound 8-1c) shows characterization data

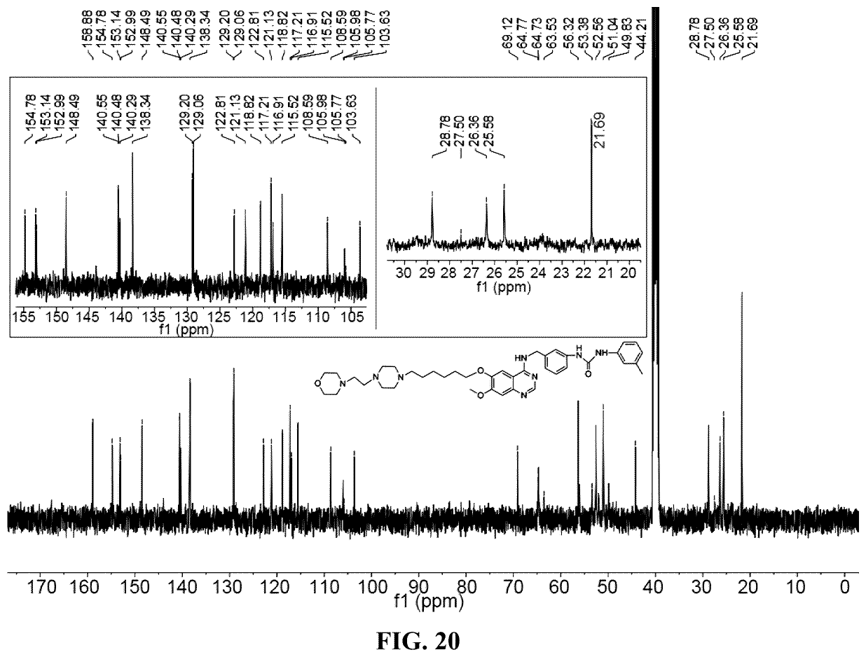

White solid was yielded (144 mg, 58% yield). The ¹³C NMR spectrum is shown in FIG. 20. m.p. 111.4-113.3° C. ¹H NMR (400 MHz, DMSO-d₆) δ 9.29 (s, 1H), 9.22 (s, 1H), 9.00 (s, 1H), 8.43 (s, 1H), 7.83 (s, 1H), 7.44 (s, 1H), 7.39 (d, J=8.1 Hz, 1H), 7.26 (s, 1H), 7.25-7.18 (m, 2H), 7.14 (s, 1H), 7.11 (d, J=7.8 Hz, 1H), 6.97 (d, J=7.6 Hz, 1H), 6.75 (d, J=7.5 Hz, 1H), 4.76 (d, J=5.5 Hz, 2H), 4.10 (t, J=6.4 Hz, 2H), 3.91 (s, 3H), 3.65 (t, J=6.8 Hz, 4H), 2.87-2.64 (m, 6H), 2.61-2.54 (m, 2H), 2.47-2.39 (m, 8H), 2.32 (t, J=7.6 Hz, 2H), 2.23 (s, 3H), 1.86-1.75 (m, 2H), 1.74-1.62 (m, 2H), 1.52-1.42 (m, 2H), 1.40-1.31 (m, 2H). ¹³C NMR (100 MHz, DMSO-d₆) δ 158.88, 154.78, 153.14, 152.99, 148.49, 140.55, 140.48, 140.29, 138.34, 129.20, 129.06, 122.81, 121.13, 118.82, 117.21, 116.91, 115.52, 108.59, 105.98, 105.77, 103.63, 69.12, 64.77, 64.73, 63.53, 56.32, 53.38, 52.56, 51.04, 49.83, 44.21, 28.78, 27.50, 26.36, 25.58, 21.69. HRMS (m/z): calcd. for C₄₀H₅₄N₈O₄ [M+H]⁺: 711.4346; found: 711.4350.

### Example 3

### N-(3-(((7-methoxy-6-((6-(4-(2-morpholinoethyl)piperazin-1-yl)hexyl)oxy)quinazolin-4-yl)amino)methyl)phenyl)-N′-(4-methylphenyl)urea (compound 8-1d) shows characterization data

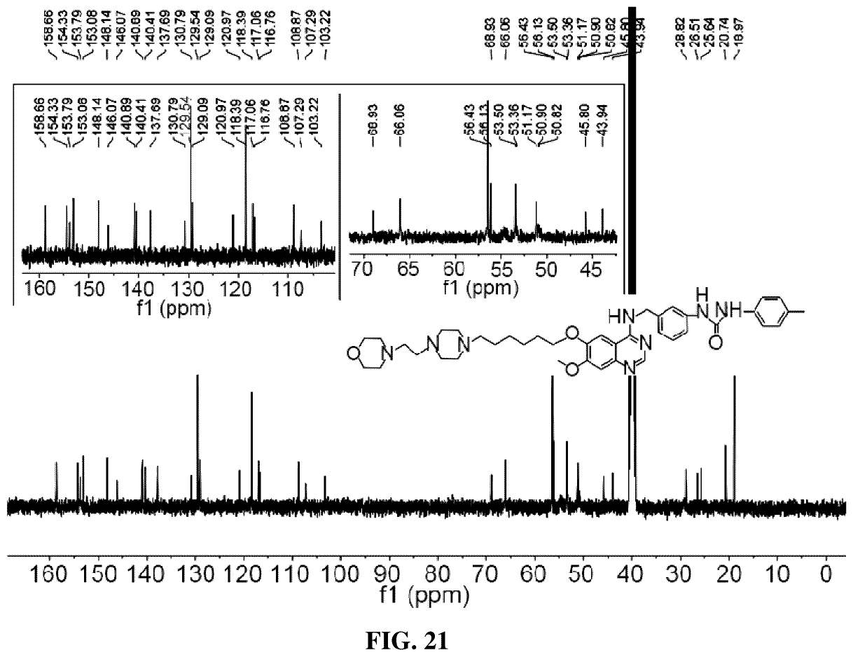

White solid was yielded (147 mg, 59% yield). The ¹³C NMR spectrum is shown in FIG. 21. m.p. 101.0-102.3° C. ¹H NMR (400 MHz, DMSO-d₆) δ 9.09 (s, 1H), 9.01 (s, 1H), 8.59 (t, J=5.6 Hz, 1H), 8.33 (s, 1H), 7.73 (s, 1H), 7.39 (d, J=8.4 Hz, 2H), 7.32 (s, 1H), 7.30 (s, 1H), 7.20 (t, J=7.8 Hz, 1H), 7.10 (s, 1H), 7.06 (s, 1H), 7.04 (s, 1H), 6.95 (d, J=7.7 Hz, 1H), 4.74 (d, J=5.6 Hz, 2H), 4.08 (t, J=6.5 Hz, 2H), 3.90 (s, 3H), 3.60 (t, J=4.8 Hz, 4H), 2.86-2.73 (m, 2H), 2.72-2.54 (m, 8H), 2.46-2.37 (m, 6H), 2.32 (t, J=7.9 Hz, 2H), 2.21 (s, 3H), 1.86-1.73 (m, 2H), 1.65-1.54 (m, 2H), 1.50-1.42 (m, 2H), 1.40-1.31 (m, 2H). ¹³C NMR (100 MHz, DMSO-d₆) δ 158.66, 154.33, 153.79, 153.08, 148.14, 146.07, 140.89, 140.41, 137.69, 130.79, 129.54, 129.09, 120.97, 118.39, 117.06, 116.76, 108.87, 107.29, 103.22, 68.93, 66.06, 56.43, 56.13, 53.50, 53.36, 51.17, 50.90, 50.82, 45.80, 43.94, 28.82, 26.51, 25.64, 20.74, 18.97. HRMS (m z): calcd. for C₄₀H₅₄N₈O₄ [M+H]⁺: 711.4346; found: 711.4371.

### Example 4

### N-(3-(((7-methoxy-6-((6-(4-(2-morpholinoethyl)piperazin-1-yl)hexyl)oxy)quinazolin-4-yl)amino)methyl)phenyl)-N′-(2-ethylphenyl)urea (compound 8-1g) shows characterization data

White solid was yielded (129 mg, 51% yield). m.p. 105.3-106.8° C. ¹H NMR (400 MHz, DMSO-d₆) δ 10.11 (s, 1H), 9.00 (s, 1H), 8.66 (s, 1H), 8.37 (s, 1H), 7.89 (s, 1H), 7.77 (dd, J=8.2, 1.4 Hz, 1H), 7.49 (d, J=2.2 Hz, 1H), 7.43 (d, J=8.2 Hz, 1H), 7.22-7.08 (m, 4H), 6.96 (ddd, J=7.3, 4.3, 1.4 Hz, 2H), 4.74 (d, J=5.7 Hz, 2H), 4.12 (t, J=6.6 Hz, 2H), 3.91 (d, J=1.9 Hz, 3H), 3.73 (d, J=4.8 Hz, 4H), 2.96-2.72 (m, 6H), 2.68-2.55 (m, 4H), 2.47-2.38 (m, 8H), 2.36 (t, J=6.3 Hz, 2H), 1.86-1.76 (m, 2H), 1.75-1.64 (m, 2H), 1.52-1.42 (m, 2H), 1.41-1.34 (m, 2H), 1.13 (t, J=7.2 Hz, 3H). ¹³C NMR (100 MHz, DMSO-d₆) δ 163.57, 158.81, 154.54, 153.68, 153.42, 148.31, 146.37, 140.81, 140.74, 137.27, 134.41, 129.06, 128.87, 126.32, 123.29, 122.39, 120.83, 117.07, 116.68, 108.83, 103.75, 69.12, 67.72, 66.23, 65.00, 60.01, 56.97, 56.24, 52.78, 50.96, 44.11, 28.81, 26.42, 25.59, 25.35, 24.51, 14.95. HRMS (m z): calcd. for C₄₁H₅₆N₈O₄ [M+H]⁺: 725.4503; found: 725.4501.

### Example 5

### N-(3-(((7-methoxy-6-((6-(4-(2-morpholinoethyl)piperazin-1-yl)hexyl)oxy)quinazolin-4-yl)amino)methyl)phenyl)-N′-(2,6-diethylphenyl)urea (compound 8-1h) shows characterization data

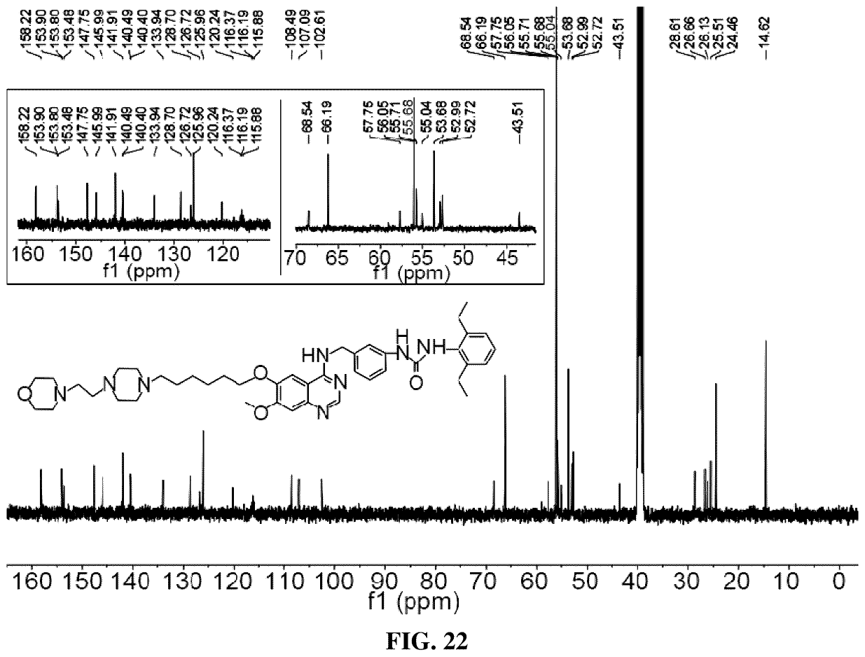

White solid was yielded (155 mg, 59% yield). The ¹³C NMR spectrum is shown in FIG. 22. m.p. 94.4-96.2° C. ¹H NMR (400 MHz, DMSO-d₆) δ 8.80 (s, 1H), 8.43 (d, J=6.1 Hz, 1H), 8.32 (s, 1H), 7.67 (s, 1H), 7.64 (s, 1H), 7.39 (d, J=8.1 Hz, 1H), 7.35 (s, 1H), 7.19 (t, J=7.7 Hz, 1H), 7.14 (d, J=7.3 Hz, 1H), 7.09 (s, 2H), 7.07 (s, 1H), 6.93 (d, J=7.6 Hz, 1H), 4.73 (s, 2H), 4.05 (t, J=6.6 Hz, 2H), 3.89 (s, 3H), 3.73 (t, J=4.6 Hz, 4H), 2.94-2.83 (m, 3H), 2.73-2.66 (m, 3H), 2.63-2.53 (m, 8H), 2.49-2.42 (m, 6H), 2.22 (t, J=7.8 Hz, 2H), 1.84-1.73 (m, 2H), 1.50-1.39 (m, 4H), 1.38-1.30 (m, 2H), 1.10 (t, J=7.5 Hz, 6H). ¹³C NMR (100 MHz, DMSO-d₆) δ 158.22, 153.90, 153.80, 153.48, 147.75, 145.99, 141.91, 140.49, 140.40, 133.94, 128.70, 126.72, 125.96, 120.24, 116.37, 116.19, 115.88, 108.49, 107.09, 102.61, 68.54, 66.19, 57.75, 56.05, 55.71, 55.68, 55.04, 53.68, 52.99, 52.72, 43.51, 28.61, 26.66, 26.13, 25.51, 24.46, 14.62. HRMS (m/z): calcd. for C₄₃H₆₀N₈O₄[M+H]⁺: 753.4816; found: 753.4816.

### Example 6

### N-(3-(((7-methoxy-6-((6-(4-(2-morpholinoethyl)piperazin-1-yl)hexyl)oxy)quinazolin-4-yl)amino)methyl)phenyl)-N′-(4-isopropylphenyl)urea (compound 8-1i) shows characterization data

White solid was yielded (142 mg, 55% yield). m.p. 176.8-177.2° C. ¹H NMR (400 MHz, DMSO-d₆) δ 9.69 (s, 1H), 9.64 (s, 1H), 8.60 (d, J=17.8 Hz, 1H), 8.36 (s, 1H), 8.31 (s, 1H), 7.75 (d, J=12.0 Hz, 1H), 7.63 (s, 1H), 7.45 (d, J=11.8 Hz, 1H), 7.36 (dd, J=8.5, 1.9 Hz, 2H), 7.17 (t, J=7.8 Hz, 1H), 7.11 (s, 1H), 7.09 (s, 1H), 6.93 (d, J=7.6 Hz, 1H), 4.72 (d, J=5.7 Hz, 2H), 3.86 (s, 2H), 3.82 (d, J=2.1 Hz, 3H), 3.73 (d, J=11.3 Hz, 4H), 2.86 (q, J=4.8 Hz, 1H), 2.82-2.65 (m, 6H), 2.64-2.53 (m, 6H), 2.47-2.40 (m, 4H), 2.24 (t, J=7.2 Hz, 2H), 1.84-1.75 (m, 2H), 1.50-1.40 (m, 4H), 1.39-1.31 (m, 2H), 1.16 (d, J=6.9 Hz, 6H). ¹³C NMR (100 MHz, DMSO-d₆) δ 159.25, 155.65, 153.28, 151.05, 149.15, 142.04, 140.63, 139.62, 138.08, 130.10, 129.22, 126.86, 121.05, 118.44, 117.28, 117.02, 107.85, 104.47, 99.23, 69.47, 63.59, 60.53, 59.93, 52.62, 52.61, 51.90, 51.07, 49.29, 49.01, 44.59, 33.22, 29.53, 28.63, 26.23, 25.45, 22.55. HRMS (m z): calcd. for C₄₂H₅₈N₈O₄ [M+H]⁺: 739.4659; found: 739.4681.

### Example 7

### N-(3-(((7-methoxy-6-((6-(4-(2-morpholinoethyl)piperazin-1-yl)hexyl)oxy)quinazolin-4-yl)amino)methyl)phenyl)-N′-(4-tert-butylphenyl)urea (Compound 8-1j) shows characterization data

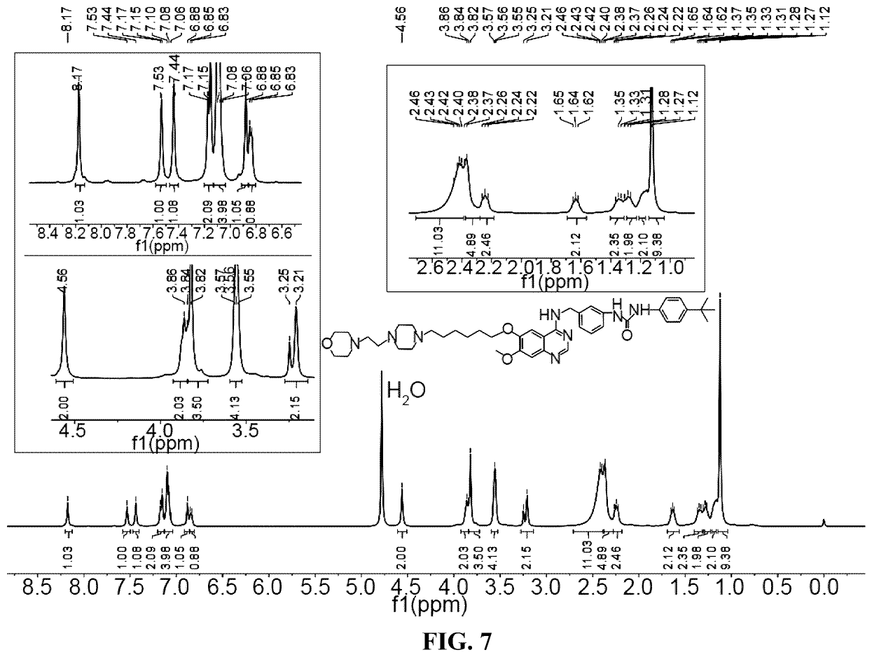

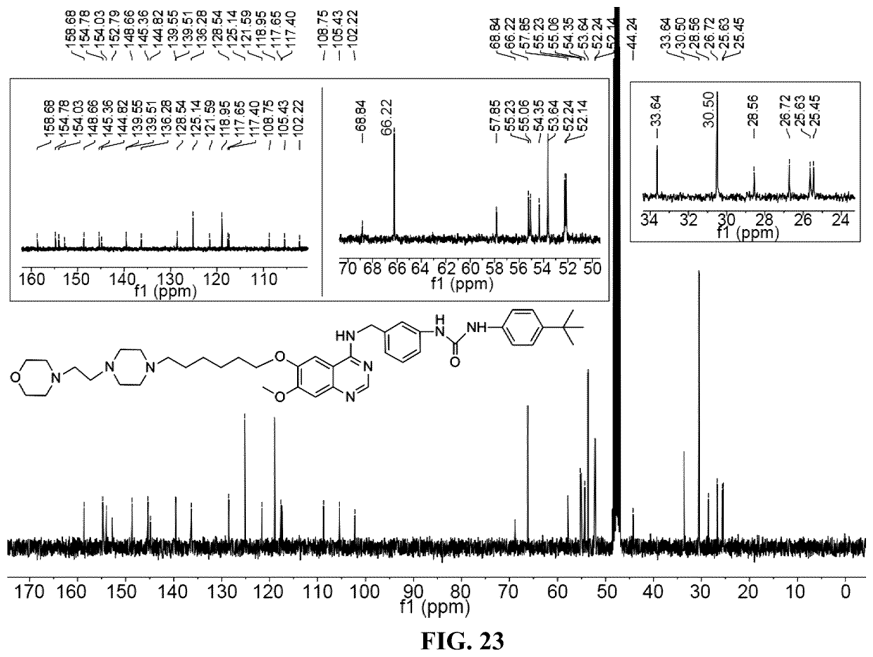

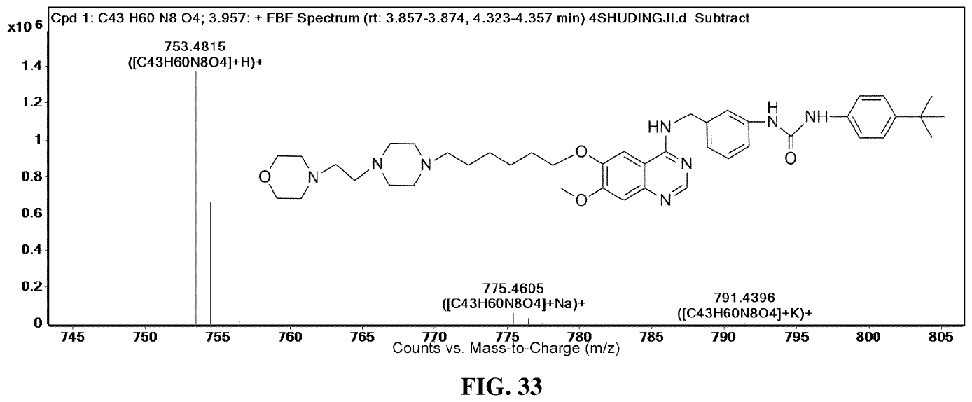

White solid was yielded (161 mg, 61% yield). ¹H NMR is shown in FIG. 7, ¹³C NMR is shown in FIG. 23, HRMS is shown in FIG. 33. m.p. 201.6-203.1° C. ¹H NMR (400 MHz, CD₃OD) δ 8.17 (s, 1H), 7.53 (s, 1H), 7.44 (s, 1H), 7.16 (d, J=8.2 Hz, 2H), 7.09 (d, J=7.8 Hz, 4H), 6.88 (s, 1H), 6.84 (d, J=6.7 Hz, 1H), 4.56 (s, 2H), 3.86 (s, 2H), 3.82 (s, 3H), 3.56 (t, J=4.7 Hz, 4H), 3.23 (d, J=7.8 Hz, 2H), 2.70-2.40 (m, 11H), 2.38-2.28 (m, 5H), 2.24 (t, J=7.9 Hz, 2H), 1.69-1.57 (m, 2H), 1.40-1.31 (m, 2H), 1.30-1.23 (m, 2H), 1.22-1.16 (m, 2H), 1.10 (s, 9H). ¹³C NMR (100 MHz, CD₃OD) δ 158.68, 154.78, 154.03, 152.79, 148.66, 145.36, 144.82, 139.55, 139.51, 136.28, 128.54, 125.14, 121.59, 118.95, 117.65, 117.40, 108.75, 105.43, 102.22, 68.84, 66.22, 57.85, 55.23, 55.06, 54.35, 53.64, 52.24, 52.14, 44.24, 33.64, 30.50, 28.56, 26.72, 25.63, 25.45. HRMS (m z): calcd. for C₄₃H₆₀N₈O₄[M+H]⁺: 753.4816; found: 753.4815.

### Example 8

### N-(3-(((7-methoxy-6-((6-(4-(2-morpholinoethyl)piperazin-1-yl)hexyl)oxy)quinazolin-4-yl)amino)methyl)phenyl)-N′-(4-fluorophenyl)urea (compound 8-11) shows characterization data

White solid was yielded (128 mg, 51% yield). m.p. 149.2-150.6° C. ¹H NMR (400 MHz, DMSO-d₆) δ 10.69 (s, 2H), 9.68 (s, 1H), 9.53 (s, 1H), 8.50 (s, 1H), 7.98 (s, 1H), 7.46-7.43 (m, 2H), 7.40-7.36 (m, 1H), 7.22 (d, J=2.0 Hz, 1H), 7.19 (d, J=7.9 Hz, 1H), 7.12-7.06 (m, 2H), 6.98 (dt, J=7.7, 1.3 Hz, 1H), 4.77 (d, J=5.7 Hz, 2H), 4.13 (t, J=6.5 Hz, 2H), 3.92 (s, 3H), 3.84 (t, J=4.8 Hz, 4H), 2.89-2.70 (m, 3H), 2.68-2.55 (m, 7H), 2.49-2.41 (m, 6H), 2.32 (t, J=4.8 Hz, 2H), 1.85-1.78 (m, 2H), 1.76-1.66 (s, 2H), 1.53-1.43 (m, 2H), 1.41-1.32 (m, 2H). ¹³C NMR (100 MHz, DMSO-d₆) δ 158.58, 158.29, 155.93, 154.68, 152.84, 151.57, 148.30, 140.00, 139.71, 136.36, 128.75, 120.69, 119.36, 119.29, 116.81, 116.49, 115.36, 115.14, 107.80, 103.65, 68.84, 67.19, 63.57, 55.97, 55.36, 55.26, 52.33, 51.62, 50.55, 43.90, 28.24, 25.83, 25.04, 23.06. HRMS (m z): calcd. for C₃₉H₅₁FN₈O₄ [M+H]⁺: 715.4096; found: 715.4085.

### Example 9

### N-(3-(((7-methoxy-6-((6-(4-(2-morpholinoethyl)piperazin-1-yl)hexyl)oxy)quinazolin-4-yl)amino)methyl)phenyl)-N′-(2-chlorophenyl)urea (compound 8-1m) shows characterization data

White solid was yielded (123 mg, 48% yield). m.p. 92.1-93.6° C. ¹H NMR (400 MHz, DMSO-d₆) δ 9.82 (s, 1H), 9.44 (s, 1H), 8.51 (s, 1H), 8.48 (s, 1H), 8.12 (dd, J=8.3, 1.3 Hz, 1H), 7.94 (s, 1H), 7.48-7.44 (m, 2H), 7.43-7.40 (d, J=1.4 Hz, 1H), 7.31-7.22 (m, 2H), 7.20 (s, 1H), 7.01 (dd, J=10.6, 4.6 Hz, 2H), 4.81 (d, J=5.6 Hz, 2H), 4.03 (t, J=6.4 Hz, 2H), 3.92 (s, 3H), 3.83 (t, J=5.8 Hz, 4H), 2.91-2.74 (m, 3H), 2.72-2.62 (m, 3H), 2.63-2.53 (m, 6H), 2.46-2.38 (m, 4H), 2.33 (t, J=6.6 Hz, 2H), 1.89-1.77 (m, 2H), 1.76-1.67 (m, 2H), 1.53-1.43 (m, 2H), 1.42-1.33 (m, 2H). ¹³C NMR (100 MHz, DMSO-d₆) δ 159.06, 157.97, 154.75, 152.70, 148.78, 145.67, 140.16, 136.46, 132.35, 129.67, 129.34, 127.96, 125.74, 123.75, 122.56, 121.89, 120.65, 117.31, 108.26, 104.13, 69.26, 64.11, 56.49, 56.44, 56.05, 52.94, 52.14, 51.07, 50.55, 45.83, 28.72, 26.30, 25.53, 23.75. HRMS (m z): calcd. for C₃₉H₅₁ClN₈O₄[M+H]⁺: 731.3800; found: 731.3813.

### Example 10

### N-(3-(((7-methoxy-6-((6-(4-(2-morpholinoethyl)piperazin-1-yl)hexyl)oxy)quinazolin-4-yl)amino)methyl)phenyl)-N′-(3,4-dichlorophenyl)urea (compound 8-1o) shows characterization data

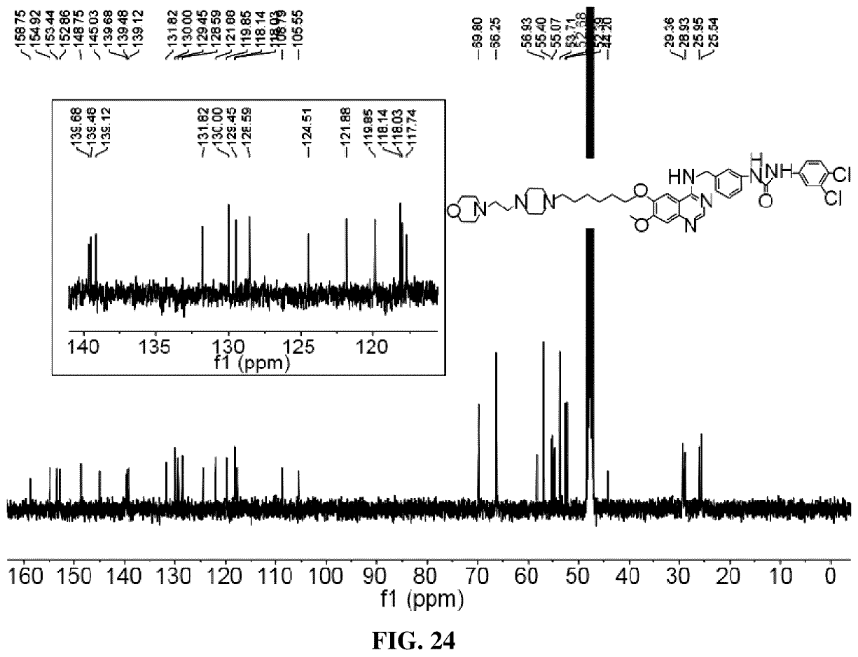

White solid was yielded (139 mg, 52% yield). The ¹³C NMR spectrum is shown in FIG. 24. m.p. 151.0-152.8° C. ¹H NMR (400 MHz, CD₃OD) δ 8.27 (s, 1H), 7.97 (d, J=2.4 Hz, 1H), 7.58 (s, 1H), 7.55 (s, 2H), 7.41 (d, J=8.8 Hz, 1H), 7.29 (d, J=7.8 Hz, 1H), 7.20 (t, J=7.8 Hz, 1H), 7.05-6.96 (m, 2H), 4.73 (s, 2H), 4.04 (t, J=6.4 Hz, 2H), 3.95 (s, 3H), 3.84 (s, 1H), 3.66 (t, J=7.8 Hz, 4H), 2.67-2.50 (m, 8H), 2.49-2.36 (m, 6H), 2.30 (t, J=7.8 Hz, 2H), 1.86-1.73 (m, 2H), 1.53-1.40 (m, 4H), 1.35-1.30 (m, 2H). ¹³C NMR (100 MHz, CD₃OD) δ 158.75, 154.92, 153.44, 152.86, 148.75, 145.03, 139.68, 139.48, 139.12, 131.82, 130.00, 129.45, 128.59, 124.51, 121.88, 119.85, 118.14, 118.03, 117.74, 108.79, 105.55, 69.80, 66.25, 58.17, 56.93, 55.40, 55.07, 54.63, 53.71, 52.69, 52.39, 44.20, 29.36, 28.93, 25.95, 25.54. HRMS (m z): calcd. for C₃₉H₅₀C₂N₈O₄[M+H]⁺: 765.3410; found: 765.3385.

### Example 11

### N-(3-(((7-methoxy-6-((6-(4-(2-morpholinoethyl)piperazin-1-yl)hexyl)oxy)quinazolin-4-yl)amino)methyl)phenyl)-N′-((4-fluoro-3-(trifluoromethyl)phenyl)urea (compound 8-1q) shows characterization data

White solid was yielded (137 mg, 50% yield). m.p. 188.8-190.5° C. ¹H NMR (400 MHz, DMSO-d₆) δ 10.05 (s, 1H), 9.55 (s, 2H), 8.53 (s, 1H), 7.99 (d, J=2.7 Hz, 1H), 7.98-7.95 (m, 1H), 7.61 (dt, J=8.3, 3.9 Hz, 1H), 7.47 (d, J=1.9 Hz, 1H), 7.44 (d, J=9.9 Hz, 1H), 7.42-7.38 (m, 1H), 7.24 (d, J=7.8 Hz, 1H), 7.22 (s, 1H), 7.06-7.00 (m, 1H), 4.80 (d, J=5.7 Hz, 2H), 4.13 (t, J=6.5 Hz, 2H), 3.93 (s, 3H), 3.65 (t, J=4.8 Hz, 4H), 2.92-2.76 (m, 3H), 2.74-2.53 (m, 9H), 2.47-2.39 (m, 4H), 2.34 (t, J=7.0 Hz, 2H), 1.86-1.78 (m, 2H), 1.76-1.68 (m, 2H), 1.52-1.44 (m, 2H), 1.42-1.34 (m, 2H). ¹³C NMR (100 MHz, DMSO-d₆) δ 159.08, 155.26, 153.22, 151.88, 148.85, 140.16, 140.06, 137.31, 130.12, 129.30, 123.97, 121.56, 118.24, 118.03, 117.56, 117.25, 115.75, 115.57, 111.05, 108.19, 106.35, 104.08, 69.31, 66.26, 58.02, 56.47, 55.64, 52.81, 52.07, 51.05, 49.46, 44.41, 28.70, 26.29, 25.51, 23.53. HRMS (m z): calcd. for C₄₀H₅₀F₄N₈O₄ [M+H]⁺: 783.3969; found: 783.3959.

### Example 12

### N-(3-(((7-methoxy-6-((6-(4-(2-morpholinoethyl)piperazin-1-yl)hexyl)oxy)quinazolin-4-yl)amino)methyl)phenyl)-N′-((4-chloro-3-(trifluoromethyl)phenyl)urea (compound 8-1r) shows characterization data

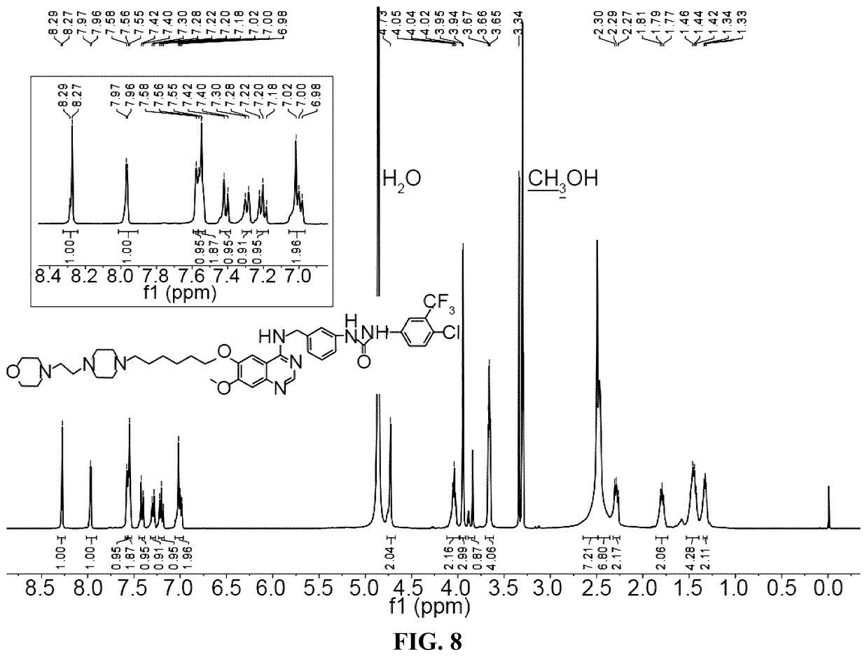

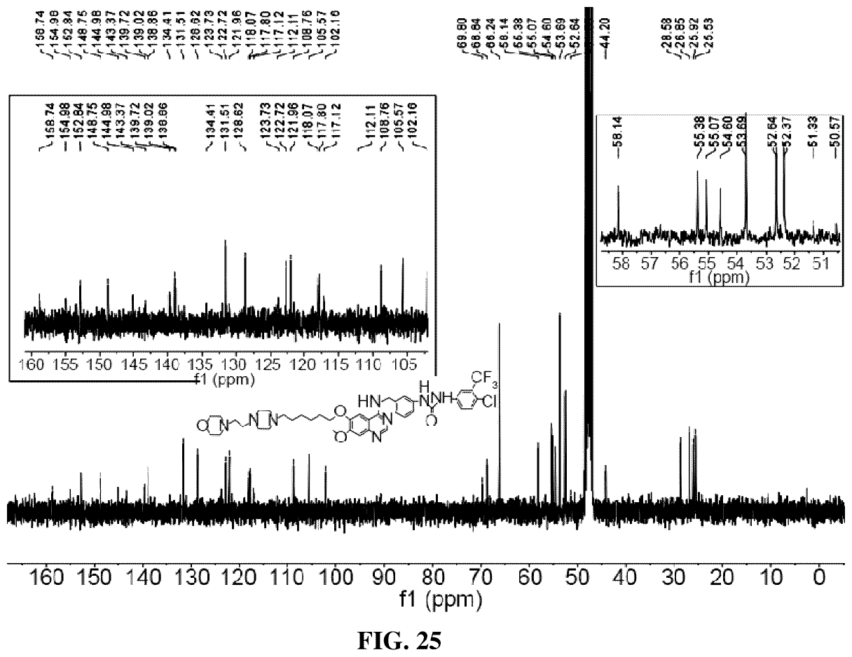

White solid was yielded (159 mg, 57% yield). ¹H NMR is shown in FIG. 8, ¹³C NMR is shown in FIG. 25. m.p. 219.3-220.7° C. ¹H NMR (400 MHz, CD₃OD) δ 8.28 (d, J=3.2 Hz, 1H), 7.97 (d, J=2.4 Hz, 1H), 7.58 (s, 1H), 7.55 (s, 2H), 7.41 (d, J=8.8 Hz, 1H), 7.29 (d, J=7.8 Hz, 1H), 7.20 (t, J=7.8 Hz, 1H), 7.04-6.96 (m, 2H), 4.73 (s, 2H), 4.04 (t, J=6.4 Hz, 2H), 3.95 (d, J=4.0 Hz, 3H), 3.84 (s, 1H), 3.66 (t, J=4.0 Hz, 4H), 2.67-2.48 (m, 7H), 2.47-2.35 (m, 7H), 2.28 (t, J=9.2 Hz, 2H), 1.86-1.73 (m, 2H), 1.53-1.39 (m, 4H), 1.35-1.30 (m, 2H). ¹³C NMR (100 MHz, CD₃OD) δ 158.74, 154.98, 152.84, 148.75, 144.98, 143.37, 139.72, 139.02, 138.86, 134.41, 131.51, 128.62, 123.73, 122.72, 121.96, 118.07, 117.80, 117.12, 112.11, 108.76, 105.57, 102.16, 69.80, 66.24, 58.14, 55.38, 55.07, 54.60, 53.69, 52.64, 51.33, 44.20, 29.41, 26.85, 25.92, 25.53. HRMS (m/z): calcd. for C₄₀H₅₀ClF₃N₈O₄ [M+H]⁺: 799.3674; found: 799.3676.

### Example 13

### N-(3-(((7-methoxy-6-((6-(4-(2-morpholinoethyl)piperazin-1-yl)hexyl)oxy)quinazolin-4-yl)amino)methyl)phenyl)-N′-(3,5-bis(trifluoromethyl)phenyl)urea (compound 8-1s) shows characterization data

White solid was yielded (146 mg, 50% yield). m.p. 201.5-202.9° C. ¹H NMR (400 MHz, DMSO-d₆) δ 9.82 (s, 1H), 9.76 (s, 1H), 9.29 (s, 1H), 8.45 (s, 1H), 8.23-8.06 (m, 2H), 7.92 (d, J=3.4 Hz, 1H), 7.60 (s, 1H), 7.57-7.46 (m, 1H), 7.39 (dd, J=7.8, 2.1 Hz, 1H), 7.26-7.15 (m, 2H), 7.03 (d, J=7.6 Hz, 1H), 4.77 (d, J=5.7 Hz, 2H), 4.11 (t, J=6.5 Hz, 2H), 3.91 (d, J=3.0 Hz, 3H), 3.81 (t, J=4.8 Hz, 4H), 2.89-2.73 (m, 3H), 2.71-2.56 (m, 9H), 2.49-2.41 (m, 4H), 2.33 (t, J=6.8 Hz, 2H), 1.85-1.76 (m, 2H), 1.74-1.65 (m, 2H), 1.52-1.42 (m, 2H), 1.41-1.32 (m, 2H). ¹³C NMR (100 MHz, DMSO-d₆) δ 158.94, 154.91, 153.09, 153.04, 148.59, 142.55, 140.59, 140.33, 139.65, 131.41, 131.08, 129.30, 125.13, 122.42, 121.84, 117.72, 117.55, 117.51, 117.30, 114.49, 108.49, 69.24, 66.25, 56.35, 55.92, 53.04, 52.24, 51.58, 51.03, 49.55, 45.67, 29.50, 28.74, 26.33, 25.54. HRMS (m z): calcd. for C₄₁H₅₀F₆N₈O₄ [M+H]⁺: 833.3937; found: 833.3931.

### Example 14

### N-(3-(((7-Methoxy-6-((6-(4-(2-morpholinoethyl)piperazin-1-yl)hexyl)oxy)quinazolin-4-yl)amino)methyl)phenyl)-N′-cyclohexylurea (compound 8-1v) shows characterization data

White solid was yielded (98 mg, 40% yield). m.p. 77.4-78.0° C. ¹H NMR (400 MHz, DMSO-d₆) δ 10.04 (s, 1H), 9.00 (s, 1H), 8.63-8.56 (m, 1H), 8.07 (d, J=15.8 Hz, 1H), 7.37 (t, J=2.2 Hz, 1H), 7.35-7.25 (m, 2H), 7.15 (q, J=7.5 Hz, 1H), 6.90 (d, J=7.6 Hz, 1H), 6.57 (s, 1H), 4.79 (d, J=5.7 Hz, 2H), 4.14 (t, J=6.6 Hz, 2H), 3.94 (d, J=3.9 Hz, 3H), 3.88 (t, J=4.7 Hz, 4H), 3.49-3.39 (m, 1H), 2.88-2.72 (m, 3H), 2.71-2.62 (m, 3H), 2.61-2.53 (m, 5H), 2.48-2.39 (m, 4H), 2.35 (t, J=7.2 Hz, 2H), 1.86-1.78 (m, 2H), 1.77-1.70 (m, 4H), 1.68-1.61 (m, 2H), 1.54-1.44 (m, 3H), 1.42-1.34 (m, 2H), 1.31-1.24 (m, 2H), 1.16-1.07 (m, 3H). ¹³C NMR (100 MHz, DMSO-d₆) δ 159.22, 155.59, 155.13, 152.91, 149.11, 146.04, 141.39, 139.42, 135.49, 132.91, 129.09, 120.24, 116.64, 107.80, 104.54, 69.49, 63.74, 56.60, 55.80, 55.79, 54.47, 52.58, 51.88, 51.15, 51.06, 49.29, 47.75, 44.58, 33.39, 28.64, 26.25, 25.76, 25.47, 24.66, 23.40. HRMS (m z): calcd. for C₃₉H₅₈N₈O₄ [M+H]⁺: 703.4659; found: 703.4651.

### Example 15

Preparation of N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-phenylurea (compound 8-2a): steps (a) to (c) were identical to those in Example 1 and thus were omitted herein. Steps (d) to (f) were performed as follows:

Step (d): 7-methoxy-N-(3-nitrobenzyl)-6-((6-(4-((4-(pyridin-2-yl)piperazin-1-yl)methyl)piperidin-1-yl)hexyl)oxy)quinazolin-4-amine (intermediate 5-2), structural formula:

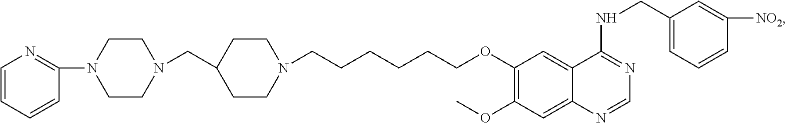 [SMILES: COc1cc2ncnc(NCc3cccc([N+](=O)[O-])c3)c2cc1OCCCCCCN1CCC(CN2CCN(c3ccccn3)CC2)CC1]

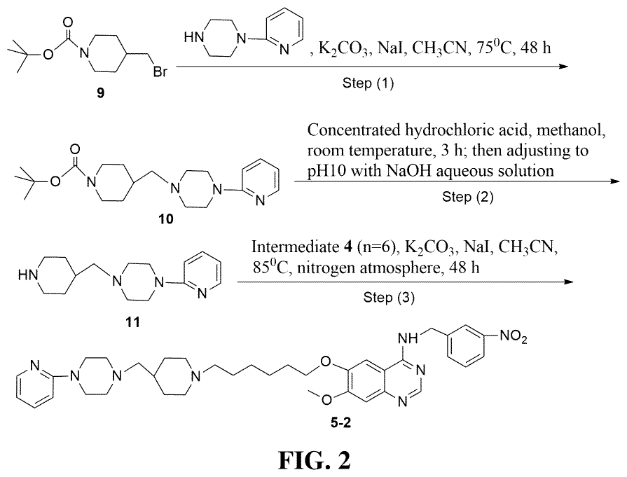

was synthesized by procedures as illustrated in FIG. 2:

Step (1): synthesis of tert-butyl 4-((4-(pyridin-2-yl)piperazin-1-yl)methyl)piperidin-1-carboxylate (intermediate 10), structural formula:

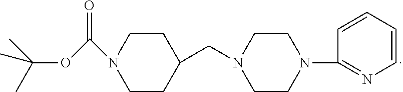 [SMILES: CC(C)(C)OC(=O)N1CCC(CN2CCN(c3ccccn3)CC2)CC1]

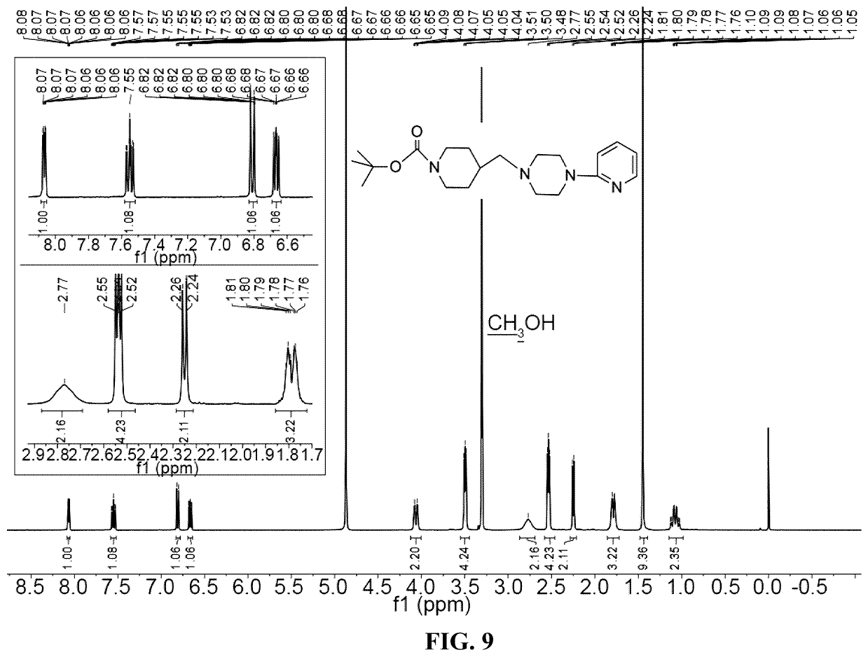

Tert-butyl 4-(bromomethyl)piperidin-1-carboxylate (9, 10.01 g, 36 mmol), 1-(2-pyridyl)piperazine (5.29 g, 32.4 mmol), K₂CO₃ (4.98 g, 36 mmol), sodium iodide (5.40 g, 36 mmol), and acetonitrile (100 mL) were added into a flask. A resulting mixture was heated to 75° C. and stirred for 48 h. After cooling to room temperature, insoluble materials were removed by filtration. A resulting filtrate was concentrated and extracted with ethyl acetate 3 times. A resulting combined organic layers were dried with anhydrous Na₂SO₄, filtered to remove desiccant, and concentrated. A resulting product was purified by silica gel column chromatography to obtain intermediate 10 as a pale yellow solid (11.94 g, 92% yield). The ¹H NMR spectrum is shown in FIG. 9. m.p. 112.6-114.1° C. ¹H NMR (400 MHz, CD₃OD) δ 8.07 (ddd, J=5.1, 2.0, 0.9 Hz, 1H), 7.55 (ddd, J=8.9, 7.1, 2.0 Hz, 1H), 6.81 (dt, J=8.7, 1.0 Hz, 1H), 6.67 (ddd, J=7.1, 5.0, 0.8 Hz, 1H), 4.06 (dt, J=13.2, 2.6 Hz, 2H), 3.54-3.46 (m, 4H), 2.77 (s, 2H), 2.59-2.47 (m, 4H), 2.25 (d, J=6.7 Hz, 2H), 1.79 (dt, J=11.5, 3.5 Hz, 3H), 1.45 (s, 9H), 1.15-1.01 (m, 2H). ¹³C NMR (100 MHz, CD₃OD) δ 159.72, 155.17, 146.98, 137.82, 113.23, 107.79, 79.48, 64.33, 58.62, 53.18, 45.04, 33.07, 30.53, 27.34. HRMS (ESI, m z): calcd. for C₂₀H₃₂N₄O₂ [M+H]⁺: 361.2604; found: 361.2598.

Step (2): synthesis of 1-(piperidin-4-yl-methyl)-4-(pyridin-2-yl)piperazine (intermediate 11), structural formula:

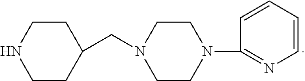 [SMILES: c1ccc(N2CCN(CC3CCNCC3)CC2)nc1]

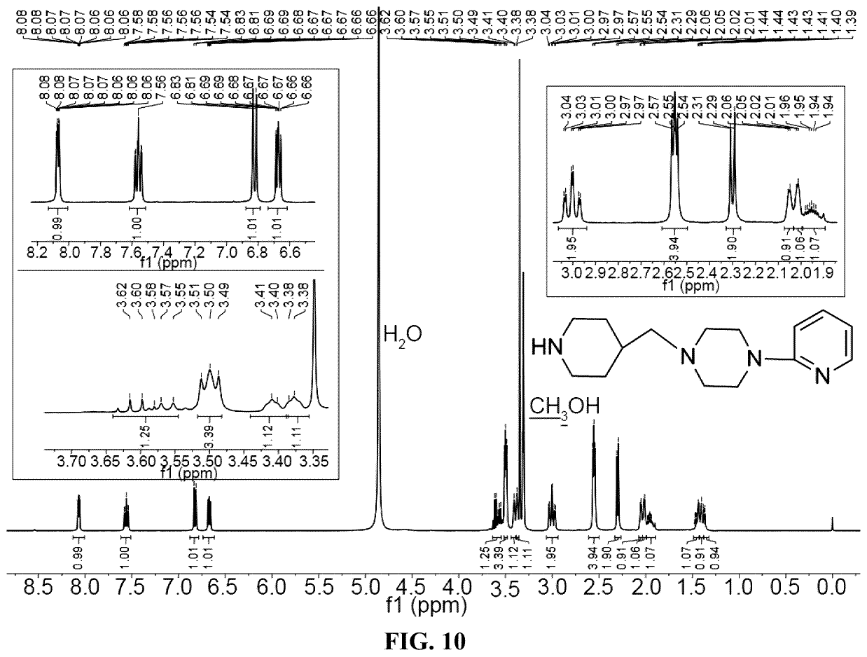

The intermediate 10 (10.81 g, 30 mmol), methanol (60 mL), and concentrated hydrochloric acid (20 mL) were added into a flask and stirred at room temperature for 3 h. A pH value was adjusted to 10 with aqueous NaOH. A resulting mixture was concentrated, filtered to remove insolubles, and a resulting filtrate was extracted with ethyl acetate 3 times. A resulting combined organic layers were dried with anhydrous Na₂SO₄, filtered, and concentrated. A resulting product was purified by silica gel column chromatography to obtain intermediate 11 as a white solid (7.03 g, 90% yield). The ¹H NMR spectrum is shown in FIG. 10. m.p. 105.6-107.3° C. ¹H NMR (400 MHz, CD₃OD) δ 8.07 (ddd, J=5.1, 2.0, 0.8 Hz, 1H), 7.56 (ddd, J=8.9, 7.1, 2.0 Hz, 1H), 6.82 (d, J=8.6 Hz, 1H), 6.67 (ddd, J=7.1, 5.1, 0.8 Hz, 1H), 3.65-3.54 (m, 1H), 3.50 (t, J=4.4 Hz, 3H), 3.41 (t, J=3.4 Hz, 1H), 3.38 (t, J=3.0 Hz, 1H), 3.00 (td, J=12.9, 3.0 Hz, 2H), 2.55 (t, J=5.1 Hz, 4H), 2.30 (d, J=7.2 Hz, 2H), 2.05 (d, J=3.6 Hz, 1H), 2.02 (d, J=3.6 Hz, 1H), 1.99-1.89 (m, 1H), 1.48-1.43 (m, 1H), 1.42-1.38 (m, 1H), 1.37-1.32 (m, 1H). ¹³C NMR (100 MHz, CD₃OD) δ 159.72, 146.97, 137.85, 113.25, 107.83, 63.43, 53.09, 48.47, 45.16, 43.77, 30.94, 27.27. HRMS (ESI, m z): calcd. for C₁₅H₂₄N₄[M+H]⁺: 261.2079; found: 261.2070.

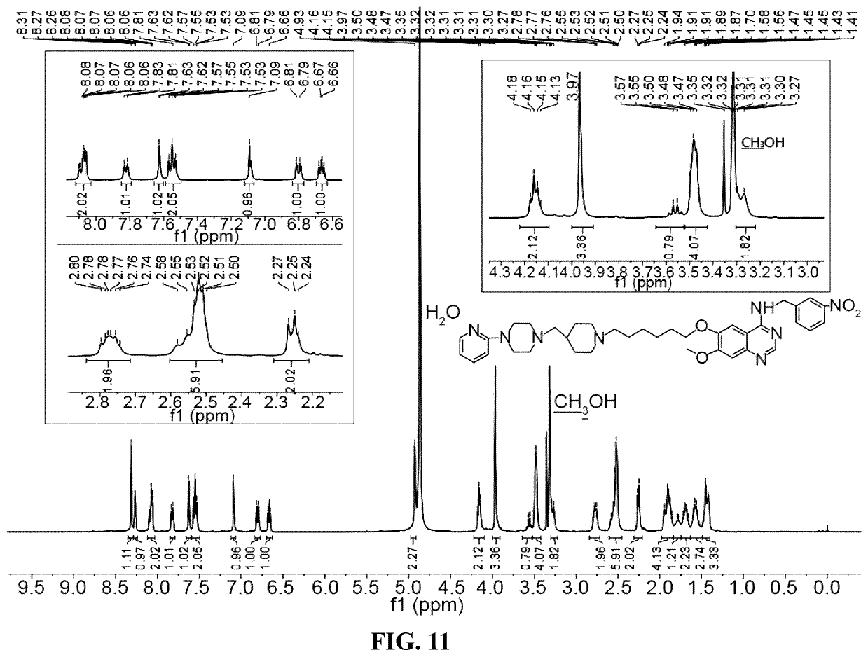

Step (3): synthesis of intermediate 5-2: the intermediate 11 (3.51 g, 13.49 mmol), the intermediate 4 (5.00 g, 11.24 mmol), K₂CO₃ (1.55 g, 11.24 mmol), NaI (1.85 g, 12.36 mmol), and acetonitrile (100 mL) were added into a flask. Under nitrogen atmosphere, a resulting mixture was heated to 85° C. under reflux and stirred for 48 h. After cooling to room temperature, insolubles were removed by suction filtration. A resulting filtrate was concentrated under reduced pressure and purified by silica gel column chromatography to obtain intermediate 5-2 as a pale yellow solid (5.64 g, 75% yield). The ¹H NMR spectrum is shown in FIG. 11. m.p. 164.2-165.9° C. ¹H NMR (400 MHz, CD₃OD) δ 8.31 (s, 1H), 8.26 (q, J=2.4 Hz, 1H), 8.13-8.02 (m, 2H), 7.82 (d, J=7.7 Hz, 1H), 7.62 (d, J=2.7 Hz, 1H), 7.55 (t, J=8.0 Hz, 2H), 7.09 (d, J=4.2 Hz, 1H), 6.80 (dd, J=8.8, 3.4 Hz, 1H), 6.67 (dd, J=7.2, 5.0 Hz, 1H), 4.93 (s, 2H), 4.15 (q, J=5.7, 5.2 Hz, 2H), 3.97 (s, 3H), 3.64-3.52 (m, 1H), 3.48 (t, J=5.4 Hz, 4H), 3.28 (d, J=7.9 Hz, 2H), 2.82-2.73 (m, 2H), 2.61-2.48 (m, 6H), 2.25 (t, J=5.7 Hz, 2H), 1.96-1.85 (m, 4H), 1.83-1.75 (m, 1H), 1.74-1.64 (m, 2H), 1.63-1.50 (m, 3H), 1.49-1.40 (m, 3H). ¹³C NMR (100 MHz, CD₃OD) δ 159.70, 158.75, 155.14, 152.93, 148.89, 148.30, 146.97, 145.20, 141.89, 137.83, 133.65, 129.27, 121.91, 121.54, 113.24, 108.74, 107.80, 105.65, 102.24, 68.90, 63.54, 59.39, 57.47, 55.15, 53.11, 52.65, 45.08, 43.32, 31.56, 28.65, 28.48, 26.44, 25.01, 25.41, 24.70. HRMS (ESI, m z): calcd. for C₃₇H₄₈N₈O₄ [M+H]⁺: 669.3877; found: 669.3869.

Step (e): synthesis of N-(3-aminobenzyl)-7-methoxy-6-((6-(4-((4-(pyridin-2-yl)piperazin-1-yl)methyl)piperidin-1-yl)hexyl)oxy)quinazolin-4-amine (intermediate 6-2), structural formula:

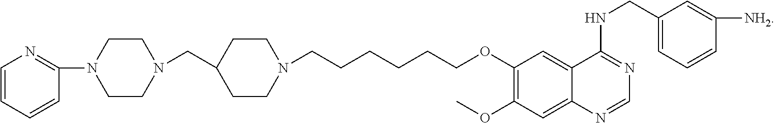 [SMILES: COc1cc2ncnc(NCc3cccc(N)c3)c2cc1OCCCCCCN1CCC(CN2CCN(c3ccccn3)CC2)CC1]

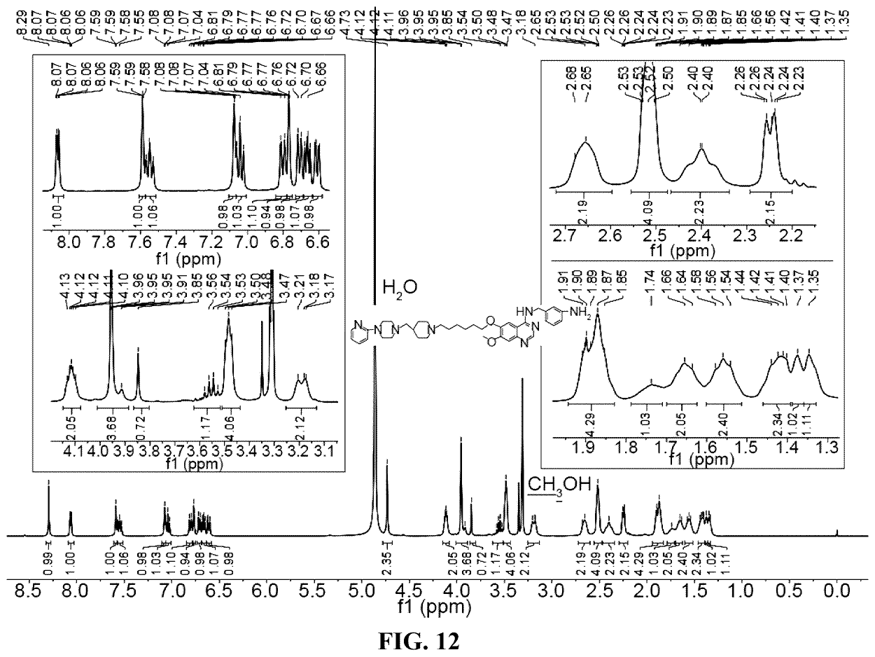

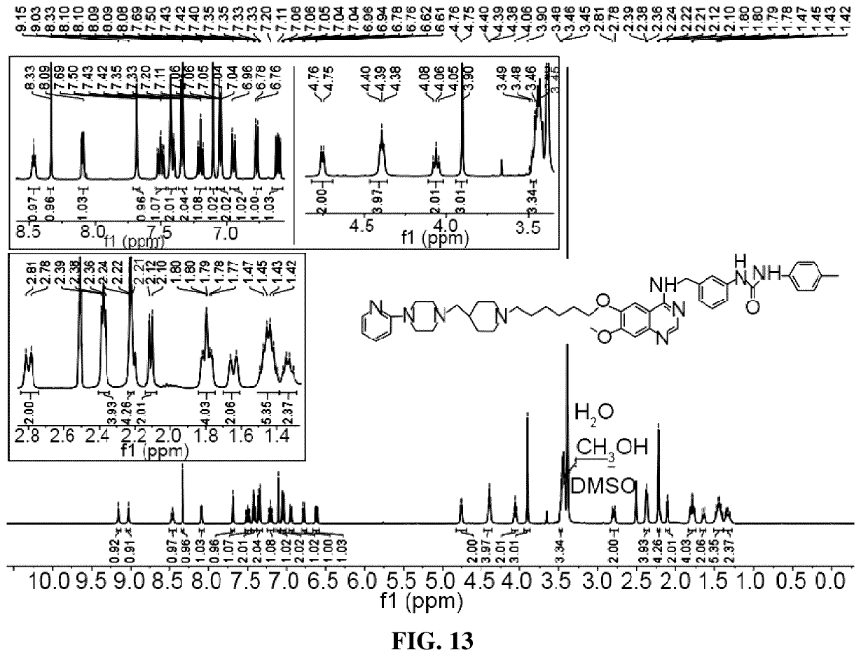

A 10 wt % aqueous ammonium chloride solution and an activated iron powder were prepared according to Step (e) of Example 1. The intermediate 5-2 (4.68 g, 7.0 mmol) and methanol (95 mL) were added into a round-bottom flask and stirred until dissolved. The activated iron powder (2.74 g, 49 mmol) and the 10 wt % aqueous ammonium chloride solution (0.56 g, 10.5 mmol) were added, followed by stirring for 5 min. A resulting mixture was heated to 60° C. and stirred for 12 h. After cooling to room temperature, insoluble materials were removed by filtration. A resulting residue was washed repeatedly with methanol. A resulting combined filtrate was concentrated, then adjusted to pH=10 with aqueous sodium carbonate solution. Extraction was conducted with ethyl acetate 3 times. Solvent was removed from a resulting combined organic layers to obtain a crude product. The crude product was purified by column chromatography to obtain intermediate 6-2 as a pale yellow solid (3.76 g, 84% yield). The ¹H NMR spectrum is shown in FIG. 12. m.p. 98.4-99.8° C. ¹H NMR (400 MHz, CD₃OD) δ 8.29 (s, 1H), 8.06 (dd, J=5.1, 1.9 Hz, 1H), 7.59 (d, J=1.7 Hz, 1H), 7.57-7.52 (m, 1H), 7.07 (dd, J=4.7, 2.6 Hz, 1H), 7.03 (d, J=7.7 Hz, 1H), 6.80 (d, J=8.7 Hz, 1H), 6.77 (d, J=2.0 Hz, 1H), 6.71 (d, J=7.5 Hz, 1H), 6.66 (dd, J=7.1, 5.0 Hz, 1H), 6.61 (dd, J=7.9, 2.3 Hz, 1H), 4.73 (s, 2H), 4.12 (dt, J=6.8, 3.7 Hz, 2H), 3.95 (t, J=4.0 Hz, 4H), 3.85 (s, 1H), 3.62-3.52 (m, 1H), 3.48 (t, J=5.0 Hz, 4H), 3.23-3.14 (m, 2H), 2.67 (d, J=9.8 Hz, 2H), 2.52 (t, J=4.9 Hz, 4H), 2.40 (t, J=8.8 Hz, 2H), 2.25 (dd, J=7.1, 2.3 Hz, 2H), 1.94-1.81 (m, 4H), 1.79-1.71 (m, 1H), 1.70-1.63 (m, 2H), 1.60-1.51 (m, 2H), 1.46-1.41 (m, 2H), 1.40-1.36 (m, 1H), 1.36-1.32 (m, 1H). ¹³C NMR (100 MHz, CD₃OD) δ 159.71, 158.84, 154.98, 152.96, 148.76, 146.97, 139.82, 137.83, 128.79, 124.99, 116.97, 114.14, 114.01, 113.23, 110.21, 108.80, 107.80, 105.61, 102.37, 68.83, 63.79, 57.75, 55.11, 53.14, 52.81, 51.12, 45.07, 44.13, 31.86, 29.00, 28.53, 26.59, 25.48, 25.03. HRMS (ESI, m/z): calcd. for C₃₇H₅₀N₈O₂[M+H]⁺: 639.4135; found: 639.4115.

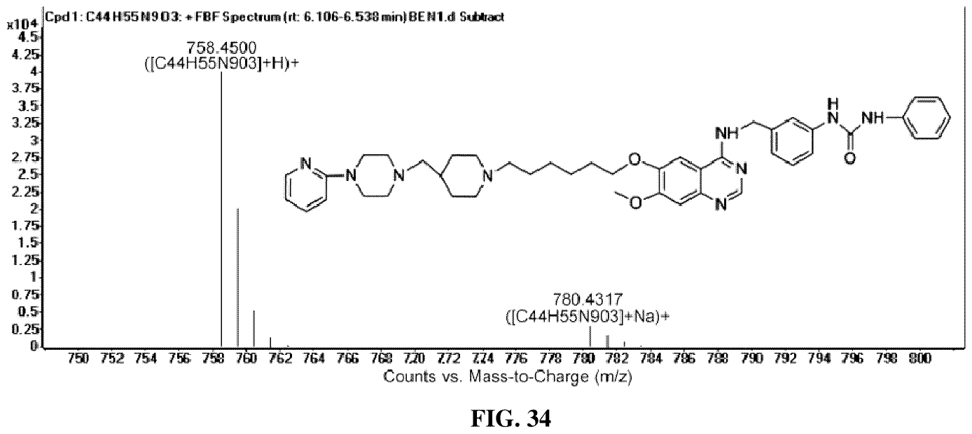

Step (f): synthesis of N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-phenylurea (compound 8-2a): dried intermediate 6-2 (224 mg, 0.35 mmol), DCM (20 mL), phenyl isocyanate (167 mg, 1.40 mmol), and triethylamine (0.5 mL) were added into a 50 mL round-bottom flask and stirred at room temperature for 72 h. Insolubles were removed by filtration. A resulting filtrate was concentrated under reduced pressure and purified by column chromatography to afford compound 8-2a as a white solid (135 mg, 51% yield). The HRMS spectrum is shown in FIG. 34. m.p. 64.2-65.9° C. ¹H NMR (400 MHz, CD₃OD) δ 8.30 (s, 1H), 8.07 (dd, J=5.0, 1.9 Hz, 1H), 7.62 (s, 1H), 7.59-7.52 (m, 2H), 7.41 (d, J=8.0 Hz, 2H), 7.33 (dd, J=7.6, 2.1 Hz, 1H), 7.27-7.20 (m, 3H), 7.07 (s, 1H), 7.03 (d, J=7.6 Hz, 1H), 6.98 (t, J=7.4 Hz, 1H), 6.80 (d, J=8.6 Hz, 1H), 6.67 (dd, J=7.1, 5.0 Hz, 1H), 4.77 (s, 2H), 4.11 (t, J=6.5 Hz, 2H), 3.96 (s, 2H), 3.87 (s, 1H), 3.84 (s, 1H), 3.69-3.63 (m, 1H), 3.63-3.57 (m, 1H), 3.54 (dd, J=6.5, 4.1 Hz, 1H), 3.48 (t, J=5.1 Hz, 3H), 3.22 (d, J=12.2 Hz, 2H), 2.70 (t, J=8.3 Hz, 2H), 2.51 (t, J=5.1 Hz, 4H), 2.23 (t, J=6.9 Hz, 2H), 2.21-2.12 (m, 1H), 1.94-1.81 (m, 4H), 1.69-1.57 (m, 4H), 1.56-1.48 (m, 2H), 1.46-1.37 (m, 4H). ¹³C NMR (100 MHz, CD₃OD) δ 162.85, 157.06, 154.66, 147.21, 145.07, 142.25, 138.79, 136.68, 136.02, 134.78, 133.56, 128.43, 127.49, 125.81, 123.21, 121.63, 120.43, 118.91, 116.82, 113.13, 110.40, 107.82, 106.94, 105.60, 103.19, 101.86, 69.80, 65.85, 64.25, 62.20, 58.05, 55.11, 54.03, 53.10, 52.65, 49.79, 45.08, 35.55, 29.38, 29.06, 28.39, 26.37, 25.33. HRMS (m z): calcd. for C₄₄H₅₅N₉O₃ [M+H]⁺: 758.4506; found: 758.4500.

According to the above procedures, appropriate starting materials and reagents were selected. By replacing phenyl isocyanate in Step (f) of Example 15 with intermediate 7-2 (general structural formula of intermediate 7-2: R₂—N═C═O), where R₂ in intermediate 7-2 corresponded to R₂ as defined in Table 2, the pyridylpiperazinylmethylpiperidinylhexyl-modified quinazolinyl-aryl urea derivatives exemplified in Examples 16 to 37 were prepared, respectively. Yields referred specifically to the yield of Step (f). Characterization data for Examples 16 to 37 are listed below.

### Example 16

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(2-methylphenyl)urea (compound 8-2b) shows characterization data

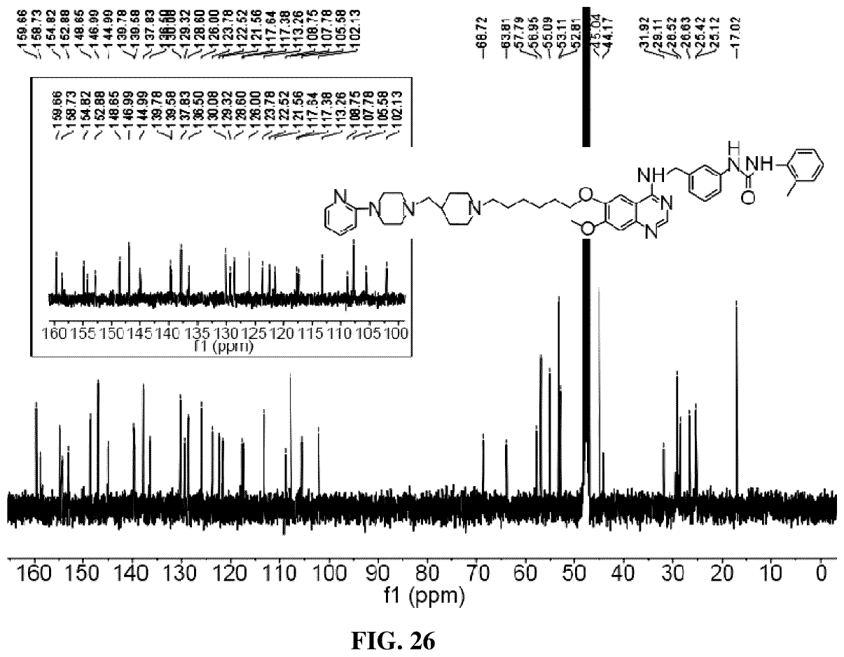

There was 42% yield. The ¹³C NMR spectrum is shown in FIG. 26. m.p. 120.0-121.8° C. ¹H NMR (400 MHz, CD₃OD) δ 8.28 (s, 1H), 8.05 (dd, J=5.1, 2.0 Hz, 1H), 7.59 (d, J=7.1 Hz, 2H), 7.53-7.47 (m, 2H), 7.27-7.16 (m, 2H), 7.11 (dd, J=7.6, 1.6 Hz, 1H), 7.03 (td, J=7.7, 1.7 Hz, 1H), 6.99 (s, 1H), 6.97-6.90 (m, 2H), 6.75 (d, J=8.6 Hz, 1H), 6.64 (dd, J=7.1, 5.0 Hz, 1H), 4.69 (s, 2H), 3.96 (t, J=6.5 Hz, 2H), 3.91 (s, 3H), 3.82 (s, 1H), 3.60 (q, J=7.1 Hz, 2H), 3.52-3.37 (m, 4H), 3.06 (d, J=11.5 Hz, 2H), 2.54-2.49 (m, 2H), 2.48-2.40 (m, 4H), 2.30-2.19 (m, 5H), 2.18-2.12 (m, 2H), 1.80 (d, J=12.6 Hz, 1H), 1.76 (dd, J=13.9, 6.8 Hz, 3H), 1.68-1.58 (m, 1H), 1.57-1.46 (m, 3H), 1.45-1.35 (m, 2H), 1.34-1.28 (m, 3H). ¹³C NMR (100 MHz, CD₃OD) δ 159.66, 158.73, 154.82, 154.27, 152.88, 148.65, 146.99, 144.99, 139.78, 139.58, 137.83, 136.50, 130.08, 129.32, 128.60, 126.00, 123.78, 122.52, 121.56, 117.64, 117.38, 113.26, 108.75, 107.78, 105.58, 102.13, 68.72, 63.81, 57.79, 56.95, 55.09, 53.11, 52.81, 45.04, 44.17, 31.92, 29.11, 28.52, 26.63, 25.42, 25.12, 17.02. HRMS (m z): calcd. for C₄₅H₅₇N₉O₃ [M+H]⁺: 772.4663; found: 772.4645.

### Example 17

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(3-methylphenyl)urea (compound 8-2c) shows characterization data

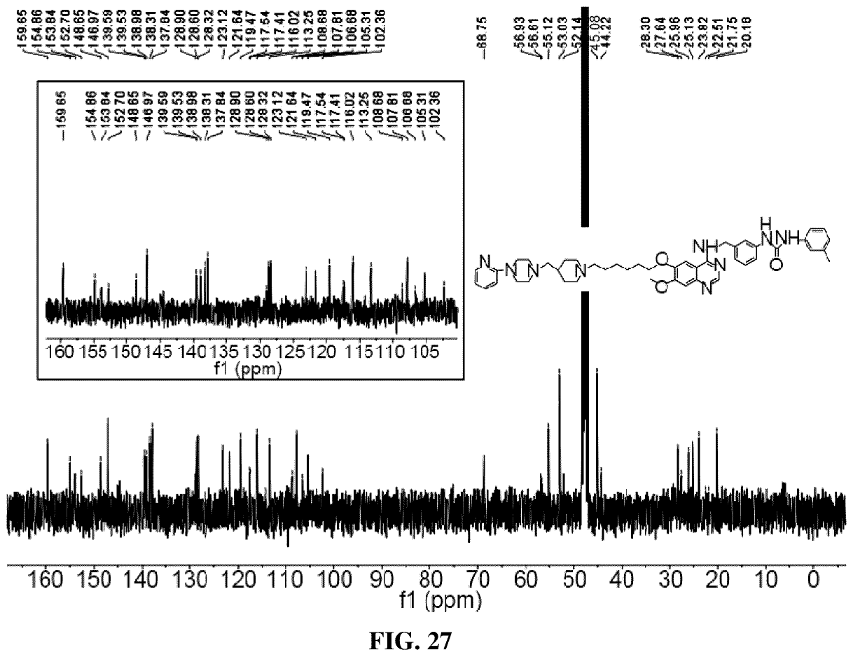

There was 49% yield. The ¹³C NMR spectrum is shown in FIG. 27. m.p. 84.5-86.2° C. ¹H NMR (400 MHz, CD₃OD) δ 8.27 (s, 1H), 8.05 (dd, J=5.2, 1.9 Hz, 1H), 7.65 (d, J=2.8 Hz, 1H), 7.61-7.57 (m, 1H), 7.56-7.48 (m, 1H), 7.28-7.12 (m, 4H), 7.06 (td, J=7.8, 2.0 Hz, 1H), 6.97 (d, J=3.3 Hz, 1H), 6.93 (d, J=7.5 Hz, 1H), 6.75 (t, J=6.7 Hz, 2H), 6.65 (t, J=6.1 Hz, 1H), 4.67 (d, J=2.7 Hz, 2H), 3.99 (d, J=6.3 Hz, 2H), 3.93 (d, J=1.8 Hz, 3H), 3.85 (s, 1H), 3.57 (dq, J=19.3, 6.8 Hz, 1H), 3.44 (d, J=5.4 Hz, 4H), 3.40-3.36 (m, 1H), 3.36-3.33 (m, 1H), 2.94-2.85 (m, 2H), 2.81 (d, J=14.9 Hz, 2H), 2.48 (q, J=4.6 Hz, 4H), 2.25-2.17 (m, 2H), 2.16-2.09 (m, 4H), 1.93 (d, J=14.6 Hz, 2H), 1.85-1.70 (m, 4H), 1.68-1.55 (m, 3H), 1.43 (q, J=12.1, 10.7 Hz, 4H). ¹³C NMR (100 MHz, CD₃OD) δ 159.65, 154.86, 153.84, 152.70, 148.65, 146.97, 139.59, 139.53, 138.98, 138.31, 137.84, 128.90, 128.60, 128.32, 123.12, 121.64, 119.47, 117.54, 117.41, 116.02, 113.25, 108.68, 107.81, 106.68, 105.31, 102.36, 68.75, 56.93, 56.61, 55.12, 53.03, 52.14, 45.08, 44.22, 28.30, 27.64, 25.96, 25.13, 23.82, 22.51, 21.75, 20.18. HRMS (m/z): calcd. for C₄₅H₅₇N₉O₃ [M+H]⁺: 772.4663; found: 772.4642.

### Example 18

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(4-methylphenyl)urea (compound 8-2d) shows characterization data

There was 44% yield. The ¹H NMR spectrum is shown in FIG. 13. m.p. 87.6-89.3° C. ¹H NMR (400 MHz, DMSO-d6) δ 9.15 (s, 1H), 9.03 (s, 1H), 8.46 (t, J=5.9 Hz, 1H), 8.33 (s, 1H), 8.09 (ddd, J=4.9, 2.0, 0.8 Hz, 1H), 7.69 (s, 1H), 7.50 (ddd, J=8.9, 7.1, 2.1 Hz, 1H), 7.44-7.39 (m, 2H), 7.37-7.32 (m, 2H), 7.20 (t, J=7.8 Hz, 1H), 7.11 (s, 1H), 7.08-7.03 (m, 2H), 6.95 (dt, J=7.6, 1.3 Hz, 1H), 6.81-6.73 (m, 1H), 6.61 (ddd, J=7.2, 4.9, 0.8 Hz, 1H), 4.75 (d, J=5.7 Hz, 2H), 4.39 (t, J=5.1 Hz, 4H), 4.06 (t, J=6.6 Hz, 2H), 3.90 (s, 3H), 3.50-3.45 (m, 3H), 2.84-2.75 (m, 2H), 2.38 (t, J=5.1 Hz, 4H), 2.25-2.20 (m, 4H), 2.11 (d, J=7.2 Hz, 2H), 1.84-1.75 (m, 4H), 1.69-1.61 (m, 2H), 1.51-1.39 (m, 5H), 1.38-1.29 (m, 2H). ¹³C NMR (100 MHz, DMSO-d6) δ 159.43, 154.79, 154.23, 152.29, 150.26, 148.61, 146.53, 146.04, 139.71, 139.26, 137.84, 137.22, 129.03, 128.71, 127.36, 126.44, 121.54, 119.52, 117.92, 113.79, 113.04, 108.62, 107.64, 107.71, 68.63, 60.52, 56.95, 56.48, 52.29, 51.36, 45.42, 44.37, 29.46, 28.84, 28.29, 27.63, 25.73, 23.24, 20.41. HRMS (m/z): calcd. for C₄₅H₅₇N₉O₃ [M+H]⁺: 772.4663; found: 772.4653.

### Example 19

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(2,4-dimethylphenyl)urea (compound 8-2e) shows characterization data

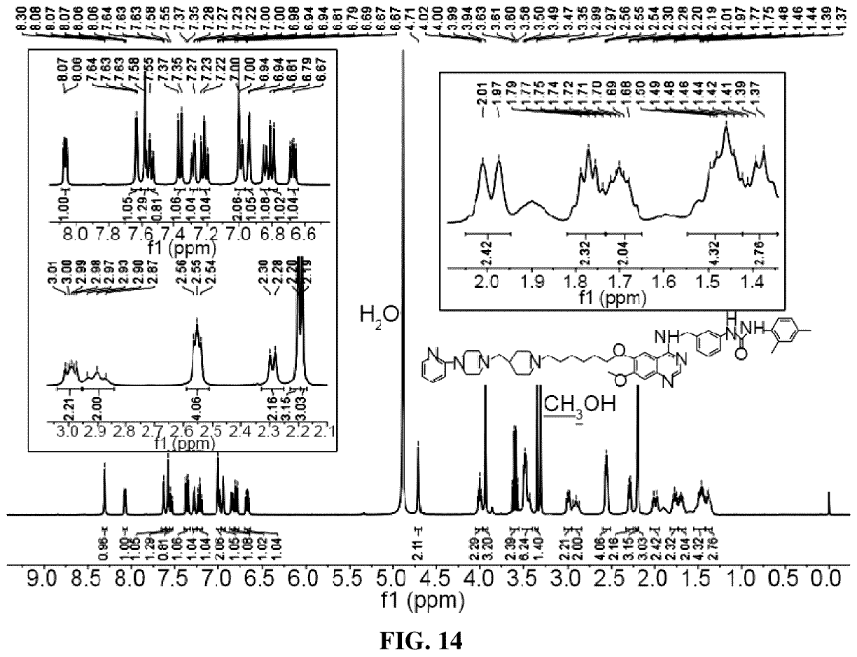

There was 50% yield. The ¹H NMR spectrum is shown in FIG. 14. m.p. 106.5-108.3° C. ¹H NMR (400 MHz, CD₃OD) δ 8.30 (s, 1H), 8.10-8.05 (m, 1H), 7.63 (d, J=1.9 Hz, 1H), 7.58 (s, 1H), 7.57-7.51 (m, 1H), 7.36 (d, J=8.1 Hz, 1H), 7.28 (dt, J=8.3, 1.4 Hz, 1H), 7.22 (t, J=7.8 Hz, 1H), 6.99 (d, J=8.4 Hz, 2H), 6.94 (d, J=2.1 Hz, 1H), 6.84 (dd, J=8.2, 2.1 Hz, 1H), 6.80 (d, J=8.7 Hz, 1H), 6.67 (dd, J=7.1, 5.0 Hz, 1H), 4.71 (s, 2H), 4.00 (t, J=6.5 Hz, 2H), 3.94 (s, 3H), 3.61 (q, J=7.0 Hz, 2H), 3.53-3.41 (m, 6H), 3.35 (s, 1H), 3.04-2.95 (m, 2H), 2.92 (d, J=12.8 Hz, 2H), 2.55 (t, J=5.0 Hz, 4H), 2.29 (d, J=7.0 Hz, 2H), 2.20 (s, 3H), 2.19 (s, 3H), 2.05-1.95 (m, 2H), 1.83-1.73 (m, 2H), 1.73-1.66 (m, 2H), 1.55-1.41 (m, 4H), 1.41-1.33 (m, 3H). ¹³C NMR (100 MHz, CD₃OD) δ 159.55, 154.736, 153.26, 152.08, 148.45, 146.05, 139.71, 139.46, 139.05, 138.52, 136.54, 128.49, 128.16, 127.35, 122.56, 121.29, 119.08, 117.25, 116.13, 112.44, 106.58, 105.46, 104.38, 103.29, 102.33, 68.45, 56.76, 56.42, 54.86, 53.18, 52.09, 45.07, 44.28, 28.48, 27.29, 25.56, 23.52, 22.62, 21.71, 20.38. HRMS (m z): calcd. for C₄₆H₅₉N₉O₃ [M+H]⁺: 786.4819; found: 786.4809.

### Example 20

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(3,5-dimethylphenyl)urea (compound 8-2f) shows characterization data

There was 42% yield. m.p. 162.8-164.5° C. ¹H NMR (400 MHz, CD₃OD) δ 8.39 (s, 1H), 8.08 (d, J=5.2 Hz, 1H), 7.63 (s, 1H), 7.56 (d, J=7.1 Hz, 1H), 7.52 (s, 1H), 7.34-7.28 (m, 2H), 7.23 (t, J=7.8 Hz, 1H), 7.11-7.03 (m, 2H), 6.94 (d, J=2.3 Hz, 1H), 6.83 (d, J=8.6 Hz, 1H), 6.69 (dd, J=7.1, 5.0 Hz, 2H), 4.69 (s, 2H), 4.07 (t, J=6.4 Hz, 2H), 3.94 (s, 1H), 3.90 (s, 3H), 3.68 (s, 2H), 3.59-3.44 (m, 6H), 2.99-2.94 (m, 2H), 2.92 (d, J=7.4 Hz, 2H), 2.64 (t, J=5.0 Hz, 4H), 2.42 (d, J=7.0 Hz, 2H), 2.34 (d, J=7.7 Hz, 6H), 1.98-1.93 (m, 2H), 1.90-1.80 (m, 2H), 1.73-1.64 (m, 2H), 1.56-1.46 (m, 4H), 1.44-1.35 (m, 3H). ¹³C NMR (100 MHz, CD₃OD) δ 159.34, 155.76, 154.72, 152.38, 150.26, 148.51, 148.44, 148.39, 139.82, 138.71, 137.26, 136.45, 134.29, 131.56, 129.67, 128.46, 120.34, 118.26, 116.58, 113.95, 113.26, 109.43, 108.39, 104.28, 69.07, 61.55, 56.81, 55.4, 52.92, 51.06, 47.09, 45.47, 30.29, 28.61, 28.03, 27.14, 25.87, 23.68, 20.59. HRMS (m/z): calcd. for C₄₆H₅₉N₉O₃ [M+H]⁺: 786.4819; found: 786.4796.

### Example 21

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(2-ethylphenyl)urea (compound 8-2g) shows characterization data

There was 47% yield. m.p. 149.6-150.9° C. ¹H NMR (400 MHz, CD₃OD) δ 8.38 (s, 1H), 8.08 (dd, J=5.1, 1.9 Hz, 1H), 7.68 (d, J=4.2 Hz, 1H), 7.59 (d, J=3.5 Hz, 2H), 7.55 (dd, J=8.1, 5.1 Hz, 1H), 7.34 (d, J=8.2 Hz, 1H), 7.24 (t, J=7.8 Hz, 1H), 7.18 (d, J=7.3 Hz, 1H), 7.10 (d, J=7.2 Hz, 2H), 7.04 (t, J=7.4 Hz, 2H), 6.83 (d, J=8.7 Hz, 1H), 6.69 (dd, J=7.1, 5.0 Hz, 1H), 4.80 (s, 2H), 4.10 (t, J=6.3 Hz, 2H), 3.96 (s, 3H), 3.69-3.57 (m, 4H), 3.54 (t, J=5.0 Hz, 4H), 3.06 (t, J=8.3 Hz, 2H), 3.03-2.93 (m, 2H), 2.71-2.60 (m, 5H), 2.40 (t, J=6.7 Hz, 2H), 2.20 (t, J=7.5 Hz, 2H), 1.89-1.82 (m, 3H), 1.78-1.69 (m, 2H), 1.56-1.49 (m, 4H), 1.46-1.41 (m, 2H), 1.42-1.33 (m, 2H), 1.19 (t, J=7.5 Hz, 3H). ¹³C NMR (100 MHz, CD₃OD) δ 159.01, 152.46, 151.61, 149.05, 146.95, 143.18, 140.58, 137.95, 136.78, 133.41, 132.63, 128.64, 125.90, 124.03, 123.69, 121.61, 120.92, 119.97, 116.38, 115.04, 111.26, 109.19, 107.91, 102.96, 102.24, 101.67, 68.88, 64.25, 60.23, 55.35, 52.89, 52.28, 48.73, 44.73, 35.98, 29.35, 29.05, 25.80, 24.64, 22.14, 21.26, 17.85. HRMS (m z): calcd. for C₄₆H₅₉N₉O₃ [M+H]⁺: 786.4819; found: 786.4809.

### Example 22

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(2,6-diethylphenyl)urea (compound 8-2h) shows characterization data

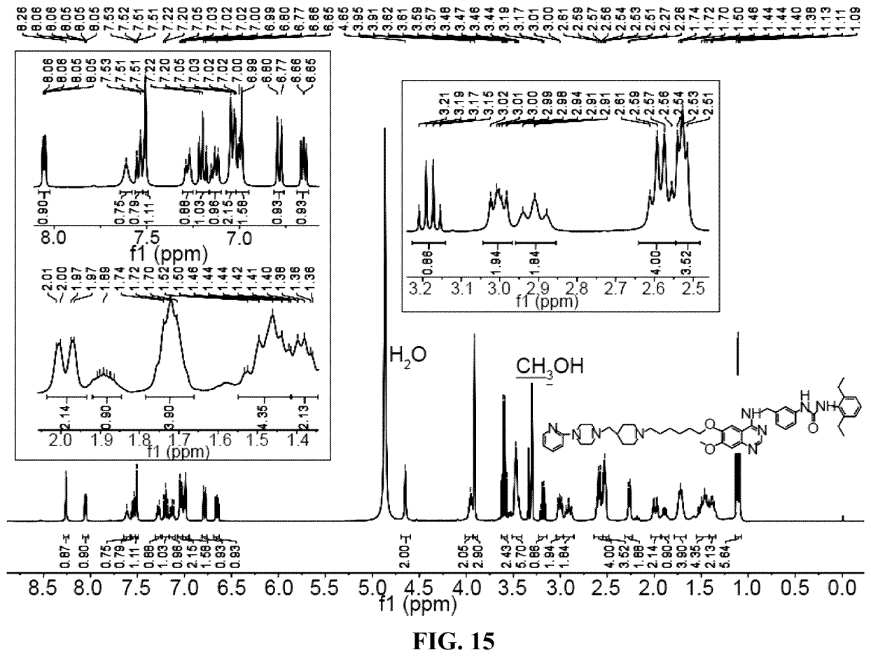

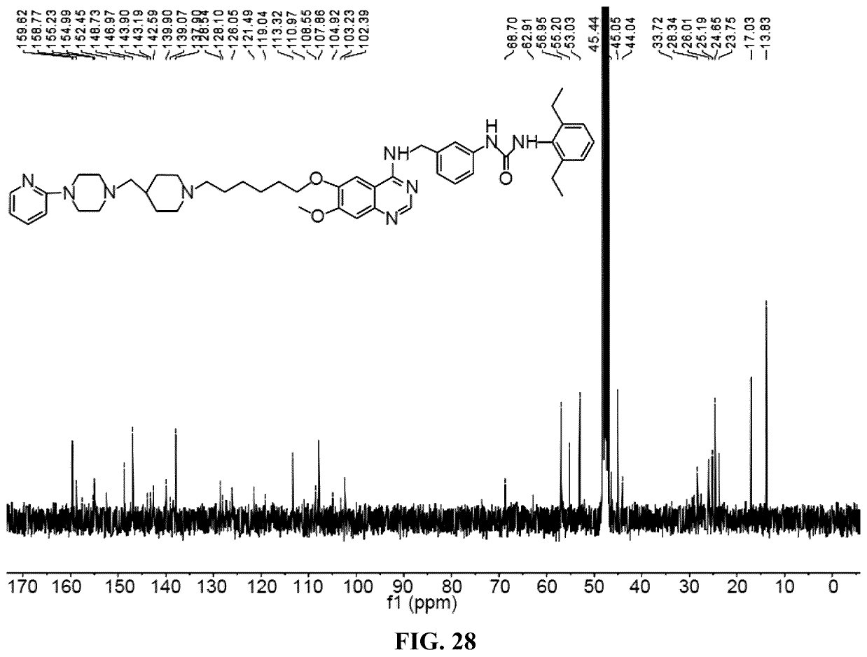

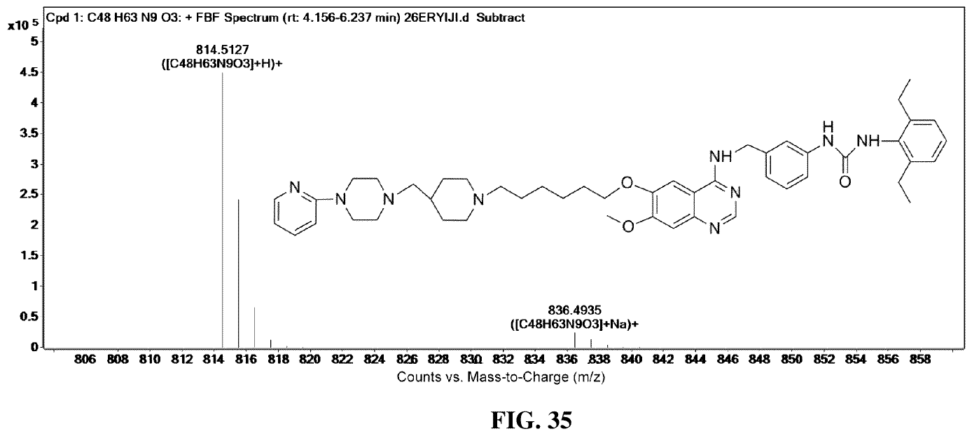

There was 49% yield. ¹H NMR is shown in FIG. 15, ¹³C NMR is shown in FIG. 28, HRMS is shown in FIG. 35. m.p. 152.6-153.8° C. ¹H NMR (400 MHz, CD₃OD) δ 8.26 (s, 1H), 8.09-8.03 (m, 1H), 7.61 (s, 1H), 7.57-7.53 (m, 1H), 7.51 (d, J=4.1 Hz, 1H), 7.28 (d, J=8.1 Hz, 1H), 7.20 (t, J=7.8 Hz, 1H), 7.16-7.11 (m, 1H), 7.07-7.02 (m, 2H), 7.00 (d, J=5.8 Hz, 2H), 6.78 (d, J=8.7 Hz, 1H), 6.65 (ddd, J=7.1, 5.0, 0.8 Hz, 1H), 4.65 (s, 2H), 3.95 (t, J=6.4 Hz, 2H), 3.91 (s, 3H), 3.60 (q, J=7.0 Hz, 2H), 3.50-3.41 (m, 6H), 3.18 (q, J=7.3 Hz, 1H), 3.05-2.97 (m, 2H), 2.96-2.86 (m, 2H), 2.64-2.56 (m, 4H), 2.53 (t, J=5.1 Hz, 4H), 2.26 (d, J=7.1 Hz, 2H), 2.04-1.95 (m, 2H), 1.92-1.84 (br, 1H), 1.72 (t, J=7.0 Hz, 4H), 1.53-1.42 (m, 4H), 1.41-1.35 (m, 2H), 1.11 (t, J=7.6 Hz, 6H). ¹³C NMR (100 MHz, CD₃OD) δ 159.62, 158.77, 157.60, 155.23, 154.99, 152.45, 148.73, 146.97, 143.90, 143.19, 142.59, 139.90, 139.07, 137.90, 128.54, 128.10, 126.05, 121.49, 119.04, 113.32, 110.97, 108.55, 107.86, 104.92, 103.23, 102.39, 68.70, 62.91, 56.95, 55.20, 53.03, 46.44, 45.05, 44.04, 33.72, 28.34, 26.01, 25.19, 24.65, 23.75, 17.03, 13.83. HRMS (m/z): calcd. for C₄₈H₆₃N₉O₃ [M+H]⁺: 814.5132; found: 814.5127.

### Example 23

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(4-isopropylphenyl)urea (compound 8-2i) shows characterization data

There was 52% yield. m.p. 63.9-65.4° C. ¹H NMR (400 MHz, CD₃OD) δ 8.45 (d, J=2.1 Hz, 1H), 8.09 (dd, J=4.9, 2.3 Hz, 1H), 7.74 (d, J=2.2 Hz, 1H), 7.63 (s, 1H), 7.61-7.55 (m, 1H), 7.30 (dt, J=8.6, 3.2 Hz, 3H), 7.26-7.21 (m, 1H), 7.12-7.07 (m, 3H), 7.03 (d, J=7.5 Hz, 1H), 6.86 (d, J=8.6 Hz, 1H), 6.71 (ddd, J=7.1, 4.9, 2.0 Hz, 1H), 4.81 (s, 2H), 4.10 (d, J=6.2 Hz, 2H), 3.99 (d, J=2.1 Hz, 3H), 3.96 (s, 1H), 3.90 (d, J=2.1 Hz, 2H), 3.70-3.58 (m, 4H), 3.09-3.03 (m, 3H), 3.02-2.97 (m, 1H), 2.88-2.83 (m, 4H), 2.82-2.77 (m, 1H), 2.58 (d, J=7.9 Hz, 2H), 2.36 (t, J=5.7 Hz, 2H), 1.90-1.81 (m, 2H), 1.80-1.71 (m, 2H), 1.62-1.49 (m, 5H), 1.48-1.40 (m, 4H), 1.18 (dd, J=7.0, 2.1 Hz, 6H). ¹³C NMR (100 MHz, CD₃OD) δ 159.24, 158.87, 157.46, 154.73, 153.66, 152.26, 148.69, 148.13, 143.71, 142.69, 140.28, 139.68, 138.84, 137.66, 128.65, 126.84, 126.17, 121.89, 119.53, 118.47, 113.52, 109.26, 108.14, 102.29, 68.75, 62.54, 56.73, 55.14, 53.47, 46.52, 45.03, 44.19, 33.96, 32.51, 29.87, 29.21, 26.45, 25.23, 24.61, 23.14. HRMS (m/z): calcd. for C₄₇H₆₁N₉O₃ [M+H]⁺: 800.4976; found: 800.4968.

### Example 24

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(4-tert-butylphenyl)urea (compound 8-2j) shows characterization data

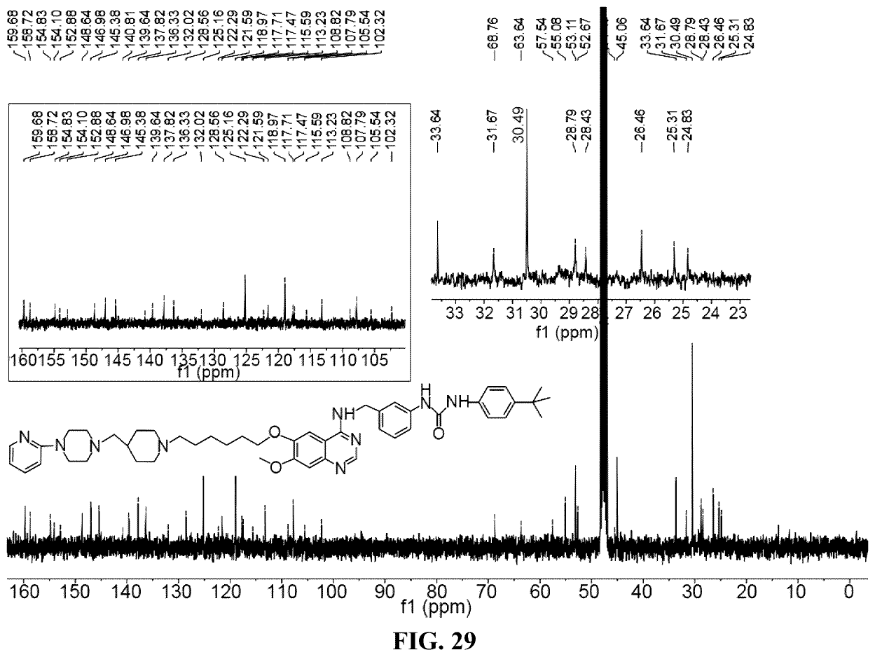

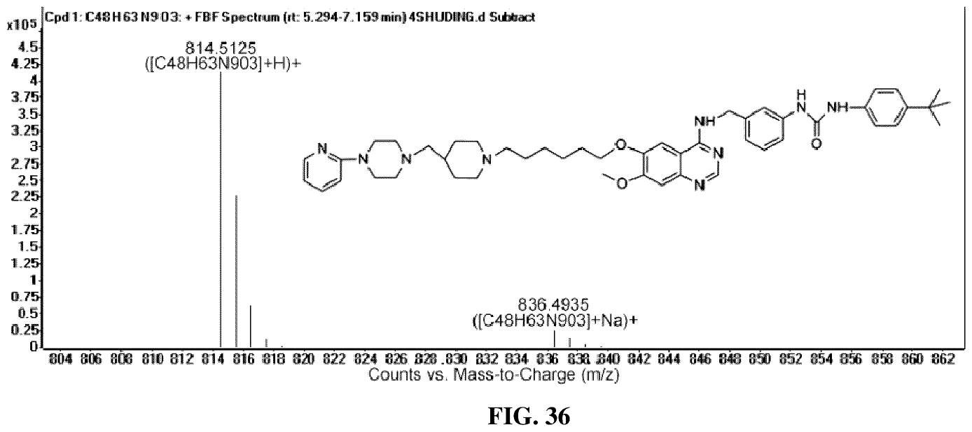

There was 58% yield. The ¹³C NMR spectrum is shown in FIG. 29. HRMS is shown in FIG. 36. m.p. 74.2-75.8° C. ¹H NMR (400 MHz, CD₃OD) δ 8.27 (s, 1H), 8.05 (dd, J=5.1, 2.0 Hz, 1H), 7.61 (d, J=1.9 Hz, 1H), 7.56 (s, 1H), 7.52 (ddd, J=8.9, 7.1, 2.0 Hz, 1H), 7.30-7.25 (m, 2H), 7.20 (dd, J=10.5, 8.3 Hz, 4H), 7.00 (s, 1H), 6.95 (d, J=7.4 Hz, 1H), 6.76 (d, J=8.6 Hz, 1H), 6.64 (dd, J=7.1, 5.0 Hz, 1H), 4.68 (s, 2H), 3.99 (t, J=6.7 Hz, 2H), 3.92 (s, 3H), 3.84 (s, 1H), 3.59-3.49 (m, 1H), 3.44 (t, J=5.1 Hz, 4H), 3.12 (d, J=11.7 Hz, 2H), 2.57 (dd, J=9.9, 6.4 Hz, 2H), 2.46 (t, J=5.1 Hz, 4H), 2.35 (t, J=11.8 Hz, 2H), 2.18 (d, J=7.1 Hz, 3H), 1.87-1.78 (m, 2H), 1.74 (q, J=7.0 Hz, 2H), 1.69-1.63 (m, 1H), 1.60-1.49 (m, 3H), 1.46-1.36 (m, 3H), 1.35-1.28 (m, 2H), 1.22 (s, 9H). ¹³C NMR (100 MHz, CD₃OD) δ 159.68, 158.72, 154.83, 154.10, 152.88, 148.64, 146.98, 145.38, 140.81, 139.64, 137.82, 136.33, 132.02, 128.56, 125.16, 122.29, 121.59, 118.97, 117.71, 117.47, 115.59, 113.23, 108.82, 107.79, 105.54, 102.32, 68.76, 63.64, 57.54, 55.08, 53.11, 52.67, 45.52, 45.06, 33.64, 31.67, 30.49, 28.79, 28.43, 26.46, 25.31, 24.83. HRMS (m/z): calcd. for C₄₈H₆₃N₉O₃ [M+H]⁺: 814.5132; found: 814.5125.

### Example 25

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(2-methoxyphenyl)urea (compound 8-2k) shows characterization data

There was 47% yield. m.p. 128.0-129.6° C. ¹H NMR (400 MHz, CD₃OD) δ 8.35 (s, 1H), 8.09-8.05 (m, 1H), 7.97-7.93 (m, 1H), 7.72 (d, J=2.1 Hz, 1H), 7.62 (s, 1H), 7.55 (ddd, J=8.9, 7.1, 2.0 Hz, 1H), 7.21 (d, J=1.9 Hz, 1H), 7.20 (d, J=1.3 Hz, 1H), 7.00 (s, 1H), 6.94-6.90 (m, 3H), 6.82-6.78 (m, 1H), 6.77 (d, J=4.0 Hz, 1H), 6.70-6.64 (m, 1H), 4.71 (s, 2H), 4.02 (t, J=6.5 Hz, 2H), 3.96 (s, 3H), 3.90 (s, 1H), 3.86 (s, 3H), 3.60 (q, J=7.0 Hz, 1H), 3.50 (t, J=5.2 Hz, 4H), 3.45-3.41 (m, 1H), 3.20 (q, J=7.3 Hz, 2H), 3.01-2.93 (m, 2H), 2.92-2.80 (m, 2H), 2.59 (t, J=5.1 Hz, 4H), 2.33 (d, J=7.0 Hz, 2H), 2.03-1.95 (m, 2H), 1.84-1.74 (m, 2H), 1.72-1.62 (m, 2H), 1.53-1.41 (m, 5H), 1.40-1.34 (m, 2H). ¹³C NMR (100 MHz, CD₃OD) δ 159.27, 155.61, 154.36, 150.47, 149.71, 149.26, 148.39, 148.06, 146.25, 139.64, 138.52, 137.66, 130.29, 129.58, 128.66, 126.71, 125.68, 122.59, 121.38, 118.36, 114.51, 113.62, 113.29, 109.38, 107.28, 105.86, 68.89, 62.34, 56.38, 55.71, 54.72, 52.93, 50.94, 46.24, 45.33, 33.49, 29.51, 29.01, 27.48, 25.96, 23.95. HRMS (m/z): calcd. for C₄₅H₅₇N₉O₄ [M+H]⁺: 788.4612; found: 788.4596.

### Example 26

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(4-methoxyphenyl)urea (compound 8-21) shows characterization data

There was 55% yield. m.p. 156.6-157.8° C. ¹H NMR (400 MHz, CD₃OD) δ 8.31 (s, 1H), 8.06 (dd, J=5.1, 1.9 Hz, 1H), 7.61 (d, J=3.9 Hz, 2H), 7.54 (ddd, J=8.9, 7.1, 2.0 Hz, 1H), 7.31-7.23 (m, 3H), 7.20 (t, J=7.8 Hz, 1H), 7.06-6.96 (m, 2H), 6.78 (t, J=8.9 Hz, 3H), 6.67 (dd, J=7.1, 5.0 Hz, 1H), 4.72 (s, 2H), 4.02 (t, J=6.6 Hz, 2H), 3.94 (s, 3H), 3.87 (s, 1H), 3.69 (s, 3H), 3.61 (q, J=7.0 Hz, 2H), 3.51-3.43 (m, 5H), 3.42 (s, 1H), 3.01-2.93 (m, 2H), 2.88 (t, J=12.7 Hz, 2H), 2.53 (t, J=5.0 Hz, 4H), 2.27 (d, J=7.0 Hz, 2H), 2.03-1.93 (m, 2H), 1.83-1.74 (m, 2H), 1.73-1.62 (m, 3H), 1.53-1.41 (m, 4H), 1.40-1.34 (m, 2H). ¹³C NMR (100 MHz, CD₃OD) δ 159.50, 155.24, 154.35, 148.92, 146.94, 145.88, 142.65, 140.19, 139.64, 139.36, 137.90, 134.93, 129.23, 128.69, 125.81, 122.29, 121.62, 121.26, 117.55, 113.67, 113.35, 112.66, 107.85, 105.88, 104.20, 102.54, 68.84, 62.77, 55.27, 54.53, 52.94, 50.88, 46.01, 44.87, 44.31, 33.26, 29.40, 28.31, 25.92, 25.13, 23.69. HRMS (m z): calcd. for C₄₅H₅₇N₉O₄ [M+H]⁺: 788.4612; found: 788.4602.

### Example 27

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(4-fluorophenyl)urea (compound 8-2n) shows characterization data

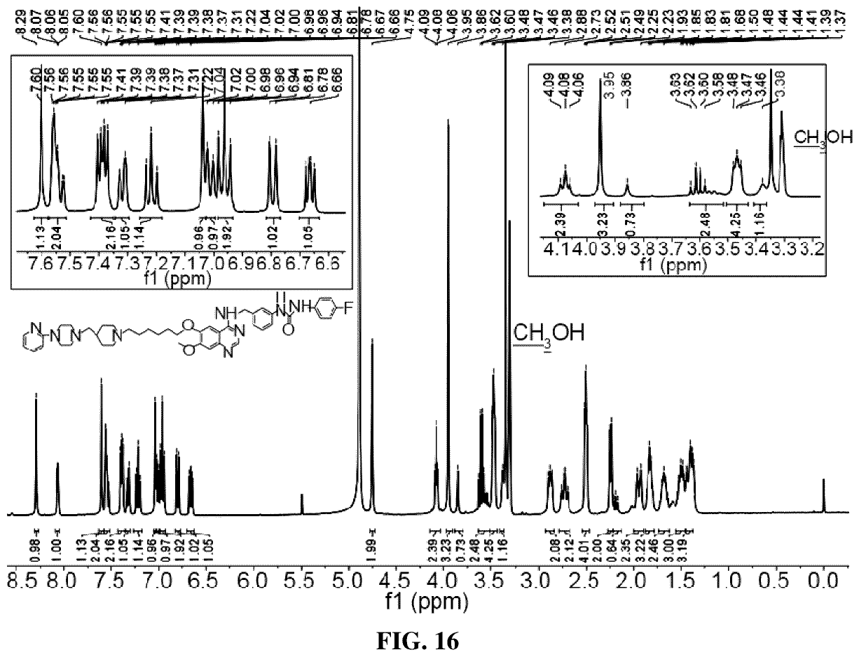

There was 56% yield. The ¹H NMR spectrum is shown in FIG. 16. m.p. 67.2-69.0° C. ¹H NMR (400 MHz, CD₃OD) δ 8.29 (s, 1H), 8.06 (dd, J=5.2, 1.9 Hz, 1H), 7.60 (s, 1H), 7.57-7.52 (m, 2H), 7.43-7.35 (m, 2H), 7.34-7.29 (m, 1H), 7.22 (t, J=7.8 Hz, 1H), 7.04 (s, 1H), 7.01 (J=7.7 Hz, 1H), 6.96 (t, J=8.8 Hz, 2H), 6.79 (d, J=8.6 Hz, 1H), 6.66 (dd, J=7.1, 5.0 Hz, 1H), 4.75 (s, 2H), 4.08 (t, J=6.5 Hz, 2H), 3.95 (s, 3H), 3.86 (s, 1H), 3.67-3.52 (m, 2H), 3.47 (t, J=5.1 Hz, 4H), 3.38 (s, 1H), 2.87 (dd, J=10.1, 6.4 Hz, 2H), 2.80-2.67 (m, 2H), 2.51 (t, J=5.0 Hz, 4H), 2.24 (d, J=7.9 Hz, 2H), 2.19 (t, J=4.2 Hz, 1H), 2.00-1.89 (m, 2H), 1.88-1.77 (m, 3H), 1.74-1.62 (m, 2H), 1.55-1.46 (m, 3H), 1.44-1.36 (m, 3H). ¹³C NMR (100 MHz, CD₃OD) δ 159.68, 158.77, 152.87, 152.29, 146.95, 143.20, 139.82, 139.37, 137.87, 137.50, 136.81, 133.44, 128.61, 125.33, 123.50, 121.69, 121.43, 120.85, 117.90, 117.67, 114.93, 114.70, 114.34, 113.27, 110.93, 107.85, 68.77, 56.94, 55.13, 53.07, 52.37, 45.10, 44.14, 41.61, 36.25, 28.35, 28.02, 26.11, 25.23, 24.13. HRMS (m z): calcd. for C₄₄H₅₄FN₉O₃[M+H]⁺: 776.4412; found: 776.4401.

### Example 28

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(2-chlorophenyl)urea (compound 8-20) shows characterization data

There was 63% yield. m.p. 89.8-91.4° C. ¹H NMR (400 MHz, CD₃OD) δ 8.28 (s, 1H), 8.07-8.01 (m, 2H), 7.61-7.57 (m, 2H), 7.54 (ddd, J=8.9, 7.1, 2.0 Hz, 1H), 7.37-7.30 (m, 2H), 7.23 (t, J=7.8 Hz, 1H), 7.17 (ddd, J=8.5, 7.4, 1.5 Hz, 1H), 7.04-6.99 (m, 2H), 6.97 (td, J=7.7, 1.6 Hz, 1H), 6.78 (d, J=8.7 Hz, 1H), 6.65 (dd, J=7.1, 5.0 Hz, 1H), 4.75 (s, 2H), 4.07 (t, J=6.5 Hz, 2H), 3.94 (s, 3H), 3.91 (s, 1H), 3.84 (s, 1H), 3.56-3.50 (m, 1H), 3.46 (t, J=5.1 Hz, 4H), 3.23 (d, J=11.9 Hz, 2H), 2.71 (t, J=8.2 Hz, 2H), 2.54-2.46 (m, 6H), 2.22 (d, J=7.1 Hz, 2H), 1.89 (d, J=4.9 Hz, 1H), 1.88-1.84 (br, 1H), 1.82 (t, J=7.1 Hz, 2H), 1.67-1.56 (m, 4H), 1.53-1.43 (m, 3H), 1.41-1.36 (m, 2H). ¹³C NMR (100 MHz, CD₃OD) δ 159.38, 156.72, 154.56, 152.28, 149.71, 148.26, 147.29, 146.87, 139.79, 137.85, 137.38, 136.66, 132.57, 130.29, 128.69, 127.98, 127.34, 124.62, 123.28, 119.59, 118.46, 113.37, 113.06, 108.23, 107.35, 105.74, 68.39, 57.82, 56.28, 55.37, 52.71, 51.36, 45.49, 44.26, 36.28, 29.71, 29.16, 27.03, 25.37, 24.06. HRMS (m z): calcd. for C₄₄H₅₄ClN₉O₃[M+H]⁺: 792.4116; found: 792.4096.

### Example 29

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(4-chlorophenyl)urea (compound 8-2q) shows characterization data

There was 53% yield. m.p. 121.4-122.9° C. ¹H NMR (400 MHz, CD₃OD) δ 8.46 (s, 1H), 8.19-8.13 (m, 2H), 7.91 (d, J=4.4 Hz, 1H), 7.63 (t, J=5.8 Hz, 2H), 7.44 (d, J=6.9 Hz, 1H), 7.35 (d, J=6.3 Hz, 1H), 7.24 (d, J=6.2 Hz, 1H), 7.16 (s, 1H), 7.08 (t, J=5.4 Hz, 2H), 6.91 (d, J=5.2 Hz, 1H), 6.86 (d, J=5.5 Hz, 1H), 6.73 (t, J=5.1 Hz, 1H), 4.75 (s, 2H), 4.07 (t, J=6.4 Hz, 2H), 4.04 (s, 1H), 3.90 (s, 3H), 3.87 (s, 1H), 3.57-3.51 (m, 1H), 3.47 (t, J=5.3 Hz, 2H), 3.23 (d, J=4.3 Hz, 2H), 3.16 (d, J=7.3 Hz, 2H), 2.78 (t, J=4.9 Hz, 2H), 2.53-2.42 (m, 6H), 2.23 (d, J=4.9 Hz, 2H), 1.96-1.89 (m, 1H), 1.84-1.79 (m, 1H), 1.75-1.65 (m, 2H), 1.60-1.52 (m, 4H), 1.51-1.43 (m, 3H), 1.38-1.31 (m, 2H). ¹³C NMR (100 MHz, CD₃OD) δ 159.35, 156.22, 154.93, 152.04, 149.68, 148.61, 148.25, 148.07, 140.26, 139.53, 137.87, 137.22, 130.09, 128.43, 127.09, 126.89, 120.74, 120.19, 118.84, 113.53, 113.29, 108.26, 107.25, 105.01, 68.59, 61.03, 56.43, 56.06, 52.73, 51.45, 46.96, 45.88, 36.48, 29.47, 29.06, 27.46, 26.07, 25.49. HRMS (m z): calcd. for C₄₄H₅₄ClN₉O₃[M+H]⁺: 792.4116; found: 792.4101.

### Example 30

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(2,4-dichlorophenyl)urea (compound 8-2r) shows characterization data

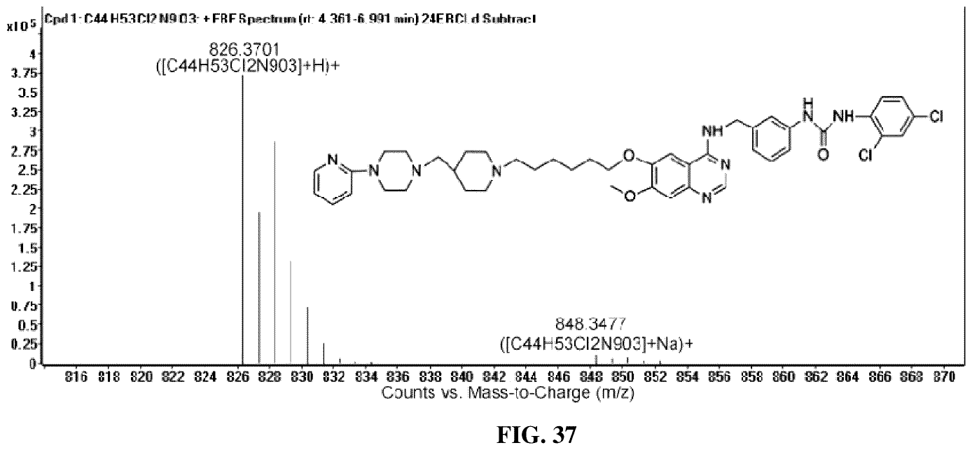

There was 46% yield. The HRMS spectrum is shown in FIG. 37. m.p. 167.2-168.8° C. ¹H NMR (400 MHz, CD₃OD) δ 8.29 (s, 1H), 8.09-8.03 (m, 1H), 7.76 (d, J=2.5 Hz, 1H), 7.58 (d, J=2.6 Hz, 2H), 7.56-7.52 (m, 1H), 7.35-7.28 (m, 2H), 7.25-7.17 (m, 2H), 7.06-6.97 (m, 2H), 6.79 (d, J=8.6 Hz, 1H), 6.66 (dd, J=7.1, 5.0 Hz, 1H), 4.74 (s, 2H), 4.05 (t, J=6.6 Hz, 2H), 3.95 (s, 3H), 3.86 (s, 3H), 3.50-3.41 (m, 4H), 3.29-3.23 (br, 1H), 2.76 (t, J=8.2 Hz, 2H), 2.59 (t, J=12.2 Hz, 2H), 2.49 (t, J=5.1 Hz, 4H), 2.22 (d, J=7.0 Hz, 2H), 2.18 (d, J=7.6 Hz, 1H), 1.96-1.86 (m, 2H), 1.85-1.74 (m, 3H), 1.68-1.56 (m, 3H), 1.52-1.42 (m, 3H), 1.40-1.34 (m, 2H). ¹³C NMR (100 MHz, CD₃OD) δ 159.21, 156.28, 154.71, 152.09, 149.92, 149.08, 148.67, 148.26, 140.06, 138.62, 137.45, 136.84, 130.73, 129.84, 129.37, 129.06, 128.56, 127.09, 123.81, 120.14, 118.87, 113.59, 113.27, 108.22, 107.36, 105.08, 69.43, 61.08, 56.43, 56.17, 52.86, 51.62, 46.68, 45.75, 36.27, 29.52, 29.06, 27.39, 26.17, 25.63. HRMS (m z): calcd. for C₄₄H₅₃Cl₂N₉O₃ [M+H]⁺: 826.3727; found: 826.3701.

### Example 31

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(2,4,6-trichlorophenyl)urea (compound 8-2s) shows characterization data

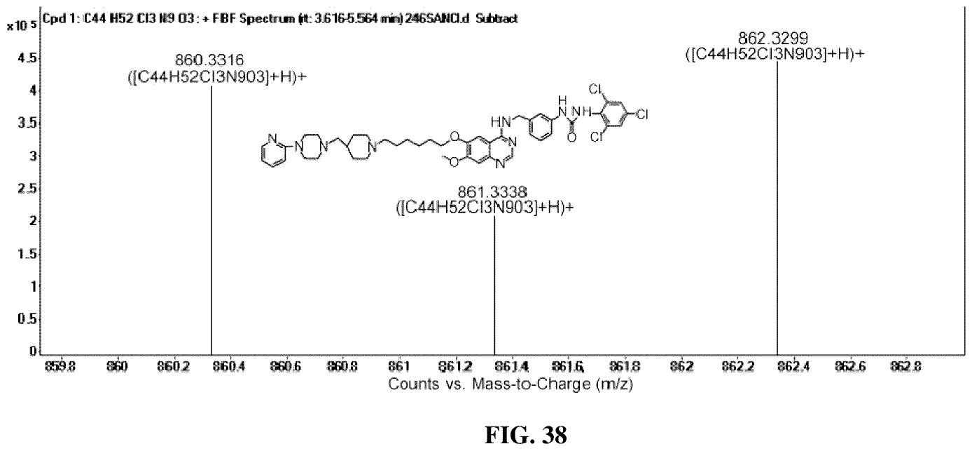

There was 58% yield. The HRMS spectrum is shown in FIG. 38. m.p. 88.4-89.9° C. ¹H NMR (400 MHz, CD₃OD) δ 8.18 (s, 1H), 7.96 (dd, J=5.1, 1.9 Hz, 1H), 7.57-7.52 (m, 1H), 7.45 (ddd, J=8.9, 7.1, 2.0 Hz, 1H), 7.37-7.33 (m, 1H), 7.28 (d, J=2.2 Hz, 1H), 7.26 (s, 1H), 7.17-7.09 (m, 2H), 6.98 (d, J=6.7 Hz, 1H), 6.93 (d, J=2.0 Hz, 1H), 6.70 (d, J=8.7 Hz, 1H), 6.56 (dd, J=7.1, 5.1 Hz, 1H), 4.63 (d, J=2.7 Hz, 2H), 3.83 (s, 3H), 3.78 (dd, J=8.1, 5.3 Hz, 2H), 3.74 (s, 1H), 3.44 (q, J=7.1 Hz, 1H), 3.24-3.19 (m, 4H), 3.12 (d, J=11.8 Hz, 2H), 2.57 (p, J=4.7 Hz, 2H), 2.41 (t, J=4.9 Hz, 4H), 2.38-2.31 (m, 2H), 2.14 (d, J=7.0 Hz, 2H), 2.11-2.00 (m, 1H), 1.86-1.75 (m, 2H), 1.71-1.59 (m, 1H), 1.58-1.50 (m, 2H), 1.50-1.42 (m, 2H), 1.34-1.26 (m, 2H), 1.25-1.20 (m, 4H). ¹³C NMR (100 MHz, CD₃OD) δ 159.46, 156.92, 154.55, 152.09, 150.23, 149.52, 148.53, 148.06, 139.91, 137.85, 137.39, 136.24, 132.69, 130.83, 129.91, 129.42, 126.55, 120.68, 119.32, 113.51, 113.25, 108.23, 107.47, 105.82, 68.73, 62.09, 56.37, 55.92, 52.91, 51.82, 46.89, 45.54, 35.97, 29.82, 29.03, 27.25, 26.03, 25.47. HRMS (m z): calcd. for C₄₄H₅₂C₁₃N₉O₃ [M+H]⁺: 860.3337; found: 860.3316.

### Example 32

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(4-(trifluoromethyl)phenyl)urea (compound 8-2t) shows characterization data

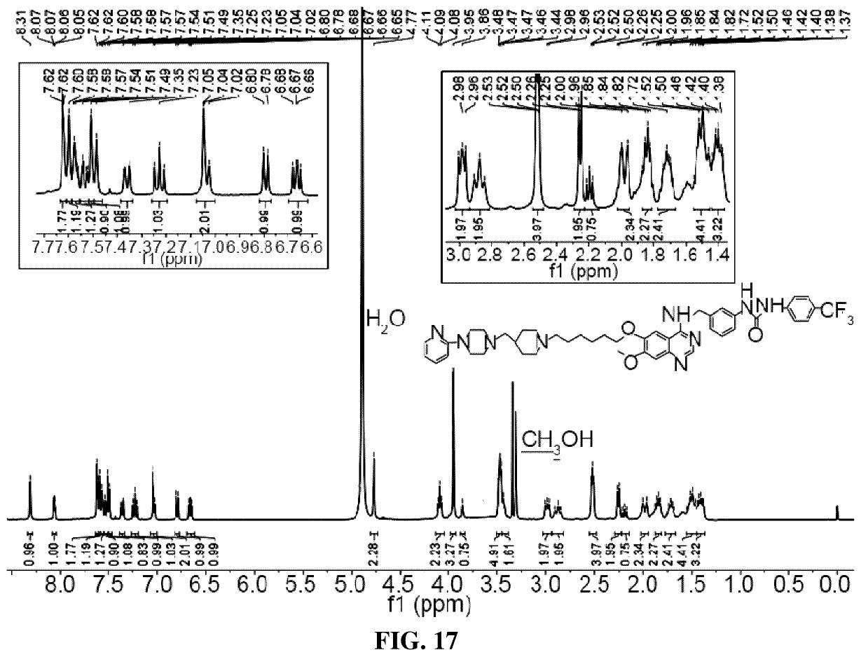

There was 38% yield. The ¹H NMR spectrum is shown in FIG. 17. m.p. 102.6-104.4° C. ¹H NMR (400 MHz, CD₃OD) δ 8.31 (s, 1H), 8.10-8.03 (m, 1H), 7.62 (d, J=2.2 Hz, 2H), 7.60 (s, 1H), 7.57 (dt, J=4.2, 1.9 Hz, 1H), 7.57-7.52 (m, 1H), 7.51 (s, 1H), 7.49 (s, 1H), 7.36 (dt, J=8.4, 1.5 Hz, 1H), 7.23 (t, J=7.8 Hz, 1H), 7.04 (d, J=9.0 Hz, 2H), 6.79 (d, J=8.7 Hz, 1H), 6.66 (dd, J=7.1, 5.0 Hz, 1H), 4.77 (s, 2H), 4.09 (t, J=6.5 Hz, 2H), 3.95 (s, 3H), 3.86 (s, 1H), 3.50-3.46 (m, 5H), 3.45-3.40 (m, 2H), 3.03-2.94 (m, 2H), 2.88 (t, J=12.6 Hz, 2H), 2.52 (t, J=5.1 Hz, 4H), 2.26 (d, J=7.1 Hz, 2H), 2.19 (t, J=7.6 Hz, 1H), 2.02-1.95 (m, 2H), 1.88-1.80 (m, 2H), 1.77-1.67 (m, 2H), 1.56-1.45 (m, 4H), 1.44-1.36 (m, 3H). ¹³C NMR (100 MHz, CD₃OD) δ 159.56, 158.24, 154.07, 152.25, 148.92, 141.25, 139.86, 139.24, 137.87, 130.53, 130.29, 130.05, 129.63, 126.54, 123.69, 121.79, 120.45, 117.96, 117.24, 115.23, 114.68, 114.23, 113.25, 110.43, 105.83, 68.73, 62.64, 55.29, 53.47, 51.78, 52.82, 45.29, 44.61, 36.03, 29.42, 28.39, 27.25, 26.61, 24.82. HRMS (m/z): calcd. for C₄₅H₅₄F₃N₉O₃[M+H]⁺: 826.4380; found: 826.4365.

### Example 33

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-((4-fluoro-3-(trifluoromethyl)phenyl)urea (compound 8-2u) shows characterization data

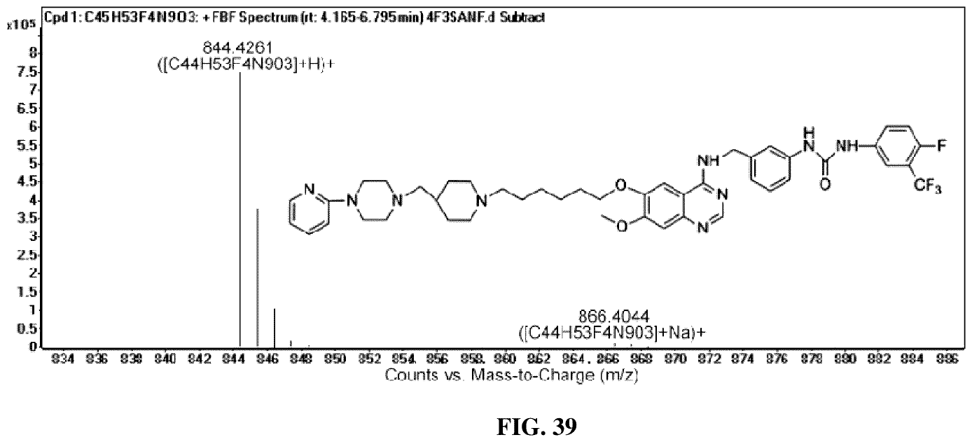

There was 52% yield. The HRMS spectrum is shown in FIG. 39. m.p. 81.2-82.7° C. ¹H NMR (400 MHz, CD₃OD) δ 8.29 (s, 1H), 8.05 (dd, J=5.3, 2.0 Hz, 1H), 7.88 (dd, J=6.3, 2.7 Hz, 1H), 7.59 (d, J=7.9 Hz, 2H), 7.57-7.51 (m, 2H), 7.35 (dt, J=8.1, 1.6 Hz, 1H), 7.27-7.22 (m, 1H), 7.21-7.17 (m, 1H), 7.05 (d, J=5.4 Hz, 1H), 7.02 (s, 1H), 6.80 (d, J=8.7 Hz, 1H), 6.66 (dd, J=7.1, 5.0 Hz, 1H), 4.77 (s, 2H), 4.10 (t, J=6.5 Hz, 2H), 3.96 (s, 3H), 3.68-3.58 (m, 1H), 3.54 (q, J=7.0 Hz, 3H), 3.47 (dd, J=6.2, 3.9 Hz, 4H), 2.90-2.78 (m, 2H), 2.66 (t, J=12.1 Hz, 2H), 2.51 (t, J=5.1 Hz, 4H), 2.25 (d, J=7.1 Hz, 2H), 2.21-2.12 (m, 1H), 1.98-1.90 (m, 2H), 1.89-1.79 (m, 3H), 1.74-1.64 (m, 3H), 1.62-1.56 (m, 2H), 1.55-1.48 (m, 3H). ¹³C NMR (100 MHz, CD₃OD) δ 159.62, 157.69, 155.38, 152.41, 151.49, 149.62, 146.88, 145.38, 140.69, 139.62, 138.26, 132.62, 128.59, 127.26, 123.78, 123.06, 122.41, 118.25, 117.36, 116.47, 114.38, 111.31, 109.43, 107.45, 105.61, 103.61, 102.36, 68.41, 60.22, 56.73, 56.02, 52.44, 51.62, 46.91, 45.34, 36.28, 29.03, 28.72, 27.53, 26.64, 25.26. HRMS (m z): calcd. for C₄₅H₅₃F₄N₉O₃[M+H]⁺: 844.4286; found: 844.4261.

### Example 34

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-((4-chloro-3-(trifluoromethyl)phenyl)urea (compound 8-2v) shows characterization data

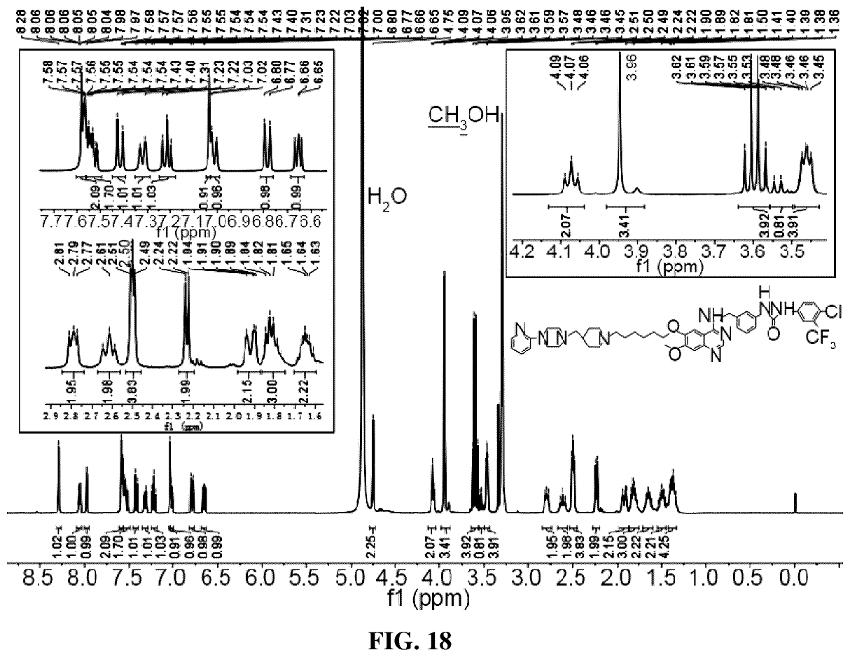

There was 57% yield. The ¹H NMR spectrum is shown in FIG. 18. m.p. 126.2-128.0° C. ¹H NMR (400 MHz, CD₃OD) δ 8.28 (s, 1H), 8.07-8.03 (m, 1H), 7.97 (d, J=2.6 Hz, 1H), 7.59-7.56 (m, 2H), 7.55-7.51 (m, 2H), 7.41 (d, J=8.8 Hz, 1H), 7.32 (dd, J=8.1, 1.9 Hz, 1H), 7.22 (t, J=7.8 Hz, 1H), 7.04 (s, 1H), 7.03-6.99 (m, 1H), 6.78 (d, J=8.6 Hz, 1H), 6.65 (ddd, J=7.3, 5.0, 0.8 Hz, 1H), 4.75 (s, 2H), 4.07 (t, J=6.5 Hz, 2H), 3.95 (s, 3H), 3.60 (q, J=7.1 Hz, 4H), 3.54 (d, J=7.0 Hz, 1H), 3.46 (dd, J=6.2, 4.0 Hz, 4H), 2.86-2.71 (m, 2H), 2.61 (t, J=12.1 Hz, 2H), 2.50 (t, J=5.1 Hz, 4H), 2.23 (d, J=7.1 Hz, 2H), 2.02-1.88 (m, 2H), 1.86-1.72 (m, 3H), 1.70-1.59 (m, 2H), 1.55-1.44 (m, 2H), 1.43-1.32 (m, 4H). ¹³C NMR (100 MHz, CD₃OD) δ 159.68, 158.70, 154.88, 152.83, 151.12, 148.67, 146.95, 144.96, 140.41, 139.71, 137.88, 131.53, 128.66, 127.96, 123.98, 122.76, 122.00, 118.04, 117.82, 115.34, 113.29, 111.79, 109.51, 107.86, 105.59, 103.28, 102.26, 68.80, 60.03, 56.95, 55.12, 53.07, 52.45, 46.38, 45.08, 44.19, 36.97, 28.37, 26.23, 25.26, 24.37, 24.11. HRMS (m z): calcd. for C₄₅H₅₃ClF₃N₉O₃ [M+H]⁺: 860.3990; found: 860.3987.

### Example 35

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-(3,5-bis(trifluoromethyl)phenyl)urea (compound 8-2w) shows characterization data

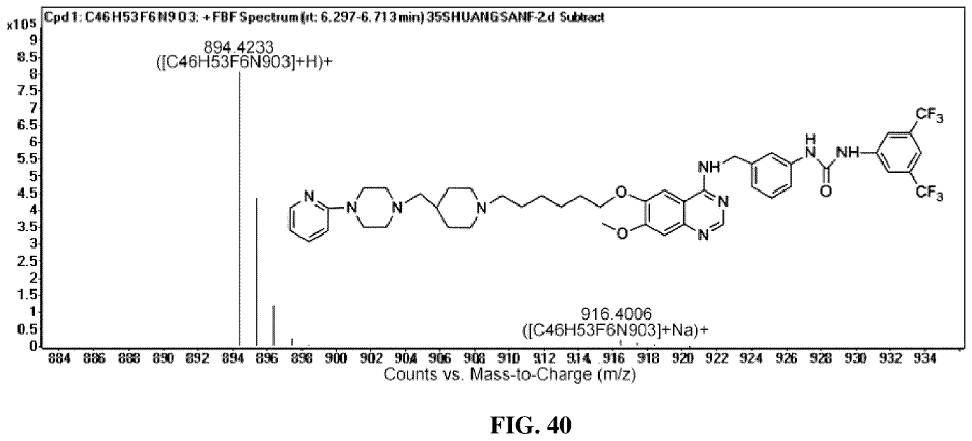

There was 44% yield. The HRMS spectrum is shown in FIG. 40. m.p. 78.6-80.3° C. ¹H NMR (400 MHz, CD₃OD) δ 8.28 (s, 1H), 8.06 (d, J=1.7 Hz, 2H), 8.04 (d, J=2.0 Hz, 1H), 7.59 (d, J=3.6 Hz, 2H), 7.54 (ddd, J=8.9, 7.1, 2.0 Hz, 1H), 7.47 (s, 1H), 7.39-7.34 (m, 1H), 7.22 (t, J=7.8 Hz, 1H), 7.05-6.99 (m, 2H), 6.79 (d, J=8.6 Hz, 1H), 6.65 (dd, J=7.1, 5.1 Hz, 1H), 4.75 (s, 2H), 4.08 (t, J=6.5 Hz, 2H), 3.95 (s, 3H), 3.84 (s, 1H), 3.58-3.49 (m, 3H), 3.46 (dd, J=6.3, 3.9 Hz, 4H), 2.83-2.74 (m, 2H), 2.61 (t, J=12.3 Hz, 2H), 2.50 (t, J=5.1 Hz, 4H), 2.24 (d, J=7.1 Hz, 2H), 2.21-2.12 (m, 1H), 1.97-1.88 (m, 2H), 1.87-1.76 (m, 3H), 1.71-1.61 (m, 3H), 1.55-1.46 (m, 3H), 1.43-1.39 (m, 2H). ¹³C NMR (100 MHz, CD₃OD) δ 159.32, 158.89, 154.64, 152.31, 150.73, 148.59, 146.37, 144.72, 140.38, 139.74, 139.16, 131.72, 129.48, 127.03, 123.69, 121.45, 120.58, 118.64, 118.02, 114.79, 113.68, 111.37, 109.42, 108.24, 105.62, 103.41, 102.29, 69.29, 60.32, 56.35, 55.87, 52.29, 51.72, 47.23, 45.72, 44.23, 35.57, 29.82, 29.03, 27.71, 26.44, 25.48. HRMS (m/z): calcd. for C₄₆H₅₃F₆N₉O₃[M+H]⁺: 894.4254; found: 894.4233.

### Example 36

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-benzylurea (compound 8-2y) shows characterization data

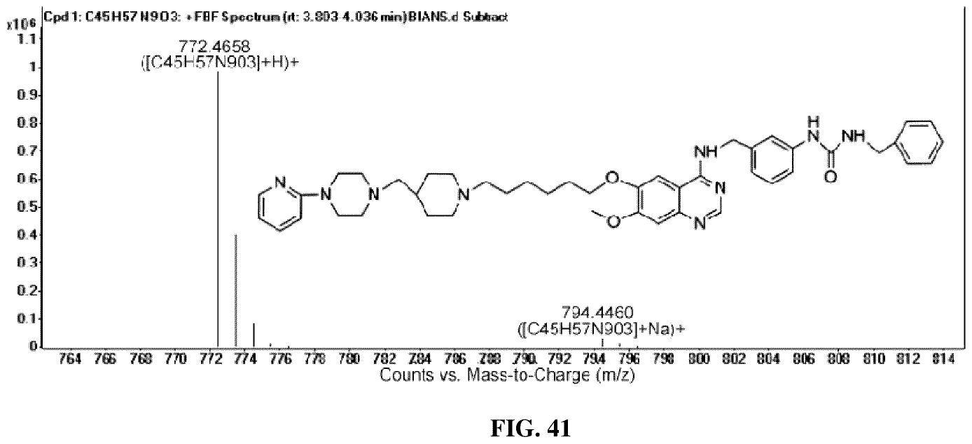

There was 56% yield. The HRMS spectrum is shown in FIG. 41. m.p. 70.8-72.3° C. ¹H NMR (400 MHz, CD₃OD) δ 8.31 (s, 1H), 8.12-8.04 (m, 1H), 7.66 (d, J=2.2 Hz, 2H), 7.56 (s, 1H), 7.52-7.46 (m, 2H), 7.37 (t, J=6.4 Hz, 1H), 7.27 (d, J=6.2 Hz, 1H), 7.21-7.15 (m, 2H), 7.14 (s, 1H), 7.04 (d, J=5.4 Hz, 1H), 6.77 (d, J=7.9 Hz, 1H), 6.62 (d, J=7.1 Hz, 1H), 4.77 (s, 2H), 4.25 (s, 2H), 4.07 (t, J=6.5 Hz, 2H), 3.95 (s, 3H), 3.85 (s, 1H), 3.59-3.48 (m, 5H), 3.43 (s, 1H), 3.05-2.95 (m, 2H), 2.87 (t, J=10.3 Hz, 2H), 2.70 (t, J=7.1 Hz, 4H), 2.61 (d, J=6.9 Hz, 2H), 2.26 (t, J=7.1 Hz, 2H), 2.21-2.12 (m, 1H), 1.92-1.83 (m, 2H), 1.81-1.75 (m, 2H), 1.72-1.63 (m, 3H), 1.61-1.50 (m, 4H), 1.43-1.31 (m, 2H). ¹³C NMR (100 MHz, CD₃OD) δ 158.79, 157.07, 155.62, 152.03, 150.49, 148.79, 148.26, 147.81, 139.62, 138.38, 137.52, 137.05, 129.33, 127.49, 127.18, 126.97, 125.65, 119.83, 118.93, 113.86, 113.05, 110.27, 107.85, 105.62, 69.85, 62.34, 56.79, 55.82, 52.68, 51.79, 47.62, 45.08, 44.23, 36.55, 29.49, 29.06, 27.68, 26.24, 25.29. HRMS (m z): calcd. for C₄₅H₅₇N₉O₃ [M+H]⁺: 772.4663; found: 772.4658.

### Example 37

### N-(3-(((7-methoxy-6-((6-(4-((4-(pyridin-2-yl)piperazin-1-yl)methyl)piperidin-1-yl)hexyl)oxy)quinazolin-4-yl)amino)methyl)phenyl)-N′-cyclohexylurea (compound 8-2z) shows characterization data

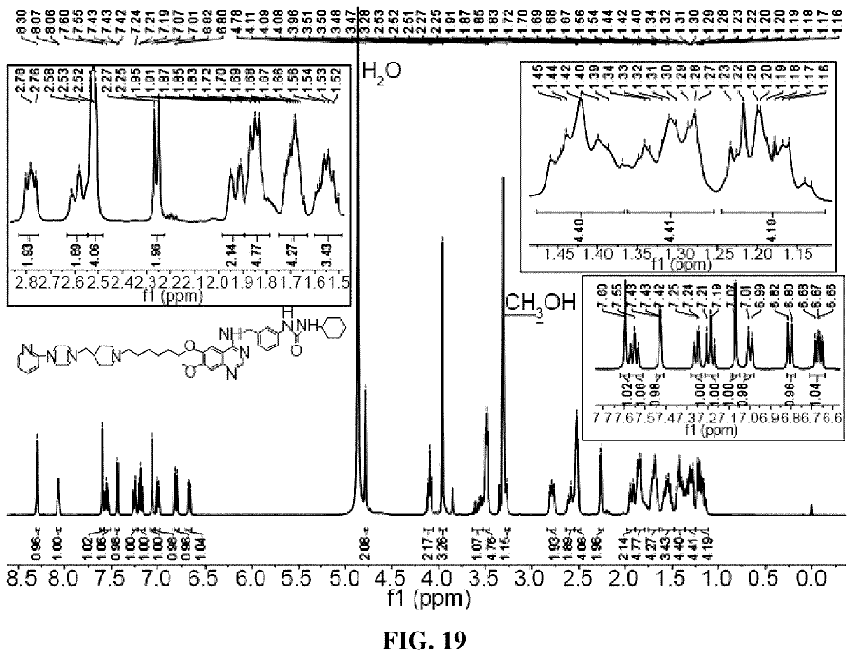

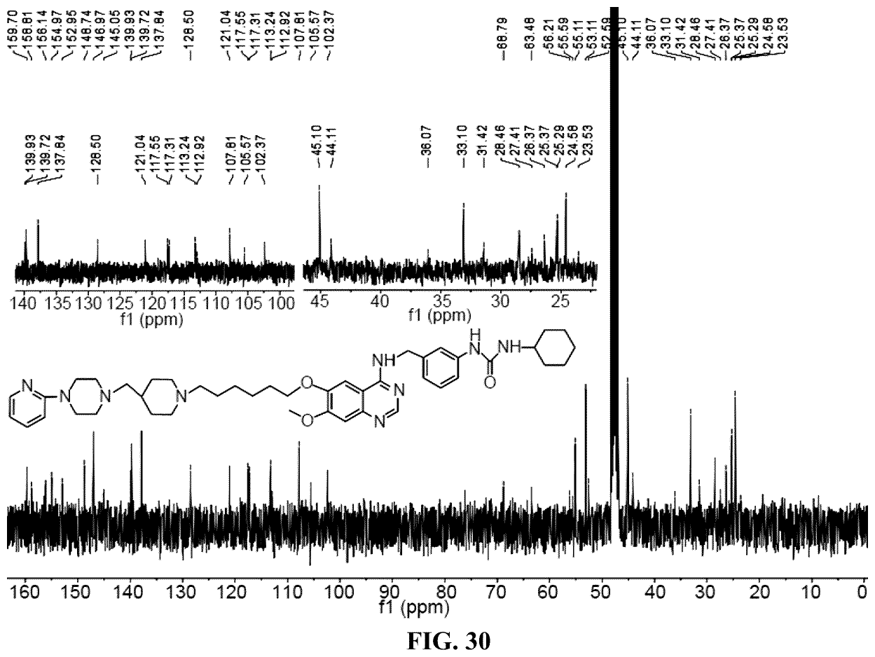

There was 41% yield. ¹H NMR is shown in FIG. 19, ¹³C NMR is shown in FIG. 30. m.p. 75.8-76.9° C. ¹H NMR (400 MHz, CD₃OD) δ 8.30 (s, 1H), 8.07 (dd, J=5.1, 2.0 Hz, 1H), 7.60 (s, 1H), 7.55 (ddd, J=8.9, 7.1, 2.0 Hz, 1H), 7.43 (t, J=1.9 Hz, 1H), 7.25 (dt, J=8.3, 1.5 Hz, 1H), 7.19 (t, J=7.8 Hz, 1H), 7.07 (s, 1H), 7.00 (d, J=7.3 Hz, 1H), 6.81 (d, J=8.7 Hz, 1H), 6.67 (dd, J=7.1, 5.0 Hz, 1H), 4.78 (s, 2H), 4.09 (t, J=6.4 Hz, 2H), 3.96 (s, 3H), 3.63-3.52 (m, 1H), 3.52-3.46 (m, 5H), 3.28 (s, 1H), 2.83-2.74 (m, 2H), 2.60 (d, J=12.2 Hz, 2H), 2.52 (t, J=5.1 Hz, 4H), 2.26 (d, J=7.1 Hz, 2H), 1.98-1.89 (m, 2H), 1.88-1.79 (m, 5H), 1.74-1.63 (m, 4H), 1.61-1.49 (m, 3H), 1.47-1.37 (m, 4H), 1.36-1.26 (m, 4H), 1.24-1.12 (m, 4H). ¹³C NMR (100 MHz, CD₃OD) δ 159.70, 158.81, 156.14, 154.97, 152.95, 148.74, 146.97, 145.05, 139.93, 139.72, 137.84, 128.50, 121.04, 117.55, 117.31, 113.24, 112.92, 107.81, 105.57, 102.37, 68.79, 63.48, 56.21, 55.59, 55.11, 53.11, 52.59, 45.10, 44.11, 36.07, 33.10, 31.42, 28.46, 27.41, 26.37, 25.37, 25.29, 24.58, 23.53. HRMS (m z): calcd. for C₄₄H₆₁N₉O₃ [M+H]⁺: 764.4976; found: 764.4951.

### Comparative Examples 1 to 17

The compounds I-1 to I-16 and II-1 to 11-17 disclosed in the inventors' prior patent application CN104292170B (“Quinazolinyl-Aryl Urea Derivatives with Antitumor Activity and Use Thereof”) were designated as Comparative Examples 1 to 17. Their structures corresponded to those listed in Table 3, with chemical structural formulas and product codes shown in Table 3.

TABLE 3
Chemical structural formulas and product codes of quinazolinyl-aryl urea derivatives

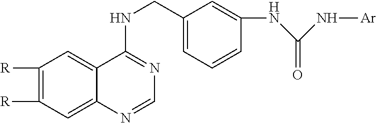

| Comparative | | | | Comparative | | | |
|---|---|---|---|---|---|---|---|
| Example | R | Ar | Product code | Example | R | Ar | Product code |
| 1 | CH₃O | 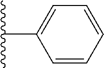 [SMILES: *c1ccccc1] | Compound I-1 | 11 | H | 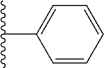 [SMILES: *c1ccccc1] | Compound II-1 |
| 2 | CH₃O | 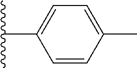 [SMILES: *c1ccc(C)cc1] | Compound I-2 | 12 | H | 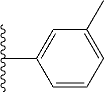 [SMILES: *c1cccc(C)c1] | Compound II-2 |
| 3 | CH₃O | 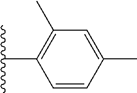 [SMILES: *c1ccc(C)cc1C] | Compound I-3 | 13 | H | 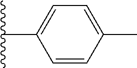 [SMILES: *c1ccc(C)cc1] | Compound II-3 |
| 4 | CH₃O | 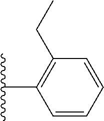 [SMILES: *c1ccccc1CC] | Compound I-5 | 14 | H | 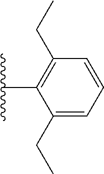 [SMILES: *c1c(CC)cccc1CC] | Compound II-5 |
| 5 | CH₃O | 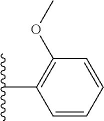 [SMILES: *c1ccccc1OC] | Compound I-6 | 15 | H | 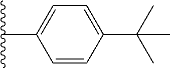 [SMILES: *c1ccc(C(C)(C)C)cc1] | Compound II-9 |
| 6 | CH₃O | 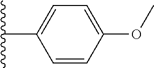 [SMILES: *c1ccc(OC)cc1] | Compound I-7 | 16 | H | 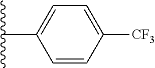 [SMILES: *c1ccc(C(F)(F)F)cc1] | Compound II-13 |
| 7 | CH₃O | 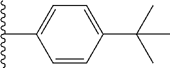 [SMILES: *c1ccc(C(C)(C)C)cc1] | Compound I-8 | 17 | H | 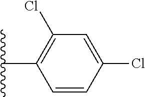 [SMILES: *c1ccc(Cl)cc1Cl] | Compound II-17 |
| 8 | CH₃O | 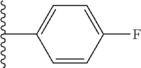 [SMILES: *c1ccc(F)cc1] | Compound I-9 | | | | |
| 9 | CH₃O | 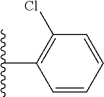 [SMILES: *c1ccccc1Cl] | Compound I-10 | | | | |
| 10 | CH₃O | 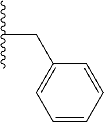 [SMILES: *Cc1ccccc1] | Compound I-14 | | | | |

### Comparative Examples 18 to 37

Compounds 8-1 to 8-23 reported in the prior publication by the inventors' research group “CHEN Chuting. Design, Synthesis and Anti-Bladder Cancer Activity of 6-Morpholinopiperidinyl-Modified Quinazolinyl-Aryl Urea Derivatives. 2022. Guangxi Normal University, MA Thesis. doi:10.27036/d.cnki.ggxsu.2022.002200” were designated as Comparative Examples 18 to 37. Their structures corresponded to those listed in Table 4, with chemical structural formulas and product codes shown therein.

TABLE 4
Chemical structural formulas and product codes of morpholinopiperidinyl-modified
quinazolinyl-aryl urea derivatives

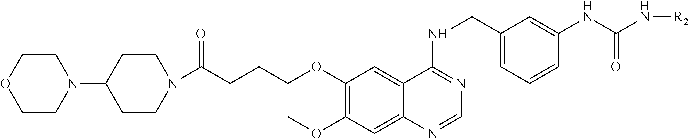

| Comparative | | | Comparative | | |
|---|---|---|---|---|---|
| Example | R₂ | Product code | Example | R₂ | Product code |
| 18 | 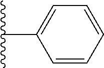 [SMILES: *c1ccccc1] | Compound 8-1 | 28 | 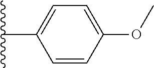 [SMILES: *c1ccc(OC)cc1] | Compound 8-11 |
| 19 | 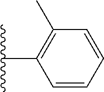 [SMILES: *c1ccccc1C] | Compound 8-2 | 29 | 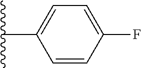 [SMILES: *c1ccc(F)cc1] | Compound 8-13 |
| 20 | 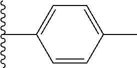 [SMILES: *c1ccc(C)cc1] | Compound 8-3 | 30 | 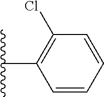 [SMILES: *c1ccccc1Cl] | Compound 8-14 |
| 21 | 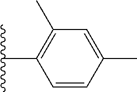 [SMILES: *c1ccc(C)cc1C] | Compound 8-4 | 31 | 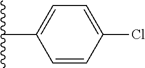 [SMILES: *c1ccc(Cl)cc1] | Compound 8-15 |
| 22 | 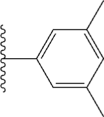 [SMILES: *c1cc(C)cc(C)c1] | Compound 8-5 | 32 | 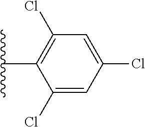 [SMILES: *c1c(Cl)cc(Cl)cc1Cl] | Compound 8-16 |
| 23 | 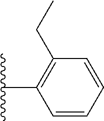 [SMILES: *c1ccccc1CC] | Compound 8-6 | 33 | 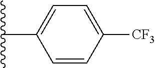 [SMILES: *c1ccc(C(F)(F)F)cc1] | Compound 8-18 |
| 24 | 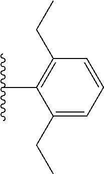 [SMILES: *c1c(CC)cccc1CC] | Compound 8-7 | 34 | 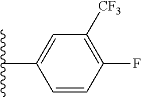 [SMILES: *c1ccc(F)c(C(F)(F)F)c1] | Compound 8-19 |
| 25 | 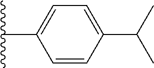 [SMILES: *c1ccc(C(C)C)cc1] | Compound 8-8 | 35 | 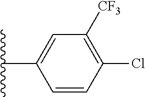 [SMILES: *c1ccc(Cl)c(C(F)(F)F)c1] | Compound 8-20 |
| 26 | 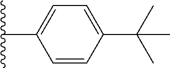 [SMILES: *c1ccc(C(C)(C)C)cc1] | Compound 8-9 | 36 | 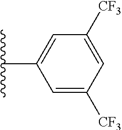 [SMILES: *c1cc(C(F)(F)F)cc(C(F)(F)F)c1] | Compound 8-21 |
| 27 | 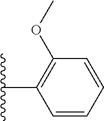 [SMILES: *c1ccccc1OC] | Compound 8-10 | 37 | 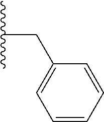 [SMILES: *Cc1ccccc1] | Compound 8-23 |

### Comparative Examples 38 to 59

Compounds 9a to 9y reported in the prior publication by the inventors' research group “HE Yuezhen. Design, Synthesis and Anti-Bladder Cancer Activity of 6-Pyridylpiperazinyl-Modified Quinazolinyl-Aryl Urea Derivatives. 2022. Guangxi Normal University, MA Thesis. doi:10.27036/d.cnki.ggxsu.2022.002175” were designated as Comparative Examples 38 to 59. Their structures corresponded to those listed in Table 5, with chemical structural formulas and product codes shown therein.

TABLE 5
Chemical structural formulas and product codes of pyridylpiperazinyl-modified
quinazolinyl-aryl urea derivatives

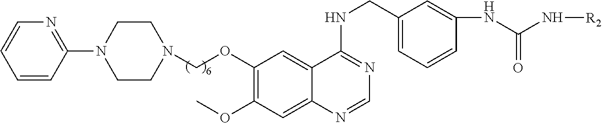

| Comparative | | | Comparative | | |
|---|---|---|---|---|---|
| Example | R₂ | Product code | Example | R₂ | Product code |
| 38 | 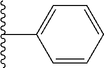 [SMILES: *c1ccccc1] | Compound 9a | 49 | 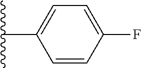 [SMILES: *c1ccc(F)cc1] | Compound 9m |
| 39 | 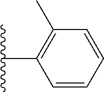 [SMILES: *c1ccccc1C] | Compound 9b | 50 | 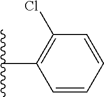 [SMILES: *c1ccccc1Cl] | Compound 9n |
| 40 | 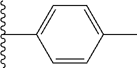 [SMILES: *c1ccc(C)cc1] | Compound 9c | 51 | 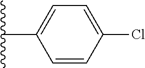 [SMILES: *c1ccc(Cl)cc1] | Compound 90 |
| 41 | 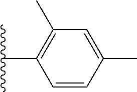 [SMILES: *c1ccc(C)cc1C] | Compound 9d | 52 | 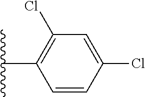 [SMILES: *c1ccc(Cl)cc1Cl] | Compound 9p |
| 42 | 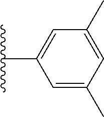 [SMILES: *c1cc(C)cc(C)c1] | Compound 9e | 53 | 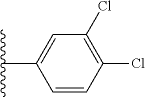 [SMILES: *c1ccc(Cl)c(Cl)c1] | Compound 9q |
| 43 | 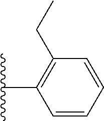 [SMILES: *c1ccccc1CC] | Compound 9f | 54 | 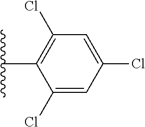 [SMILES: *c1c(Cl)cc(Cl)cc1Cl] | Compound 9r |
| 44 | 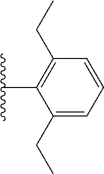 [SMILES: *c1c(CC)cccc1CC] | Compound 9g | 55 | 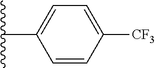 [SMILES: *c1ccc(C(F)(F)F)cc1] | Compound 9t |
| 45 | 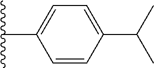 [SMILES: *c1ccc(C(C)C)cc1] | Compound 9h | 56 | 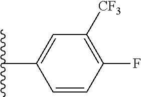 [SMILES: *c1ccc(F)c(C(F)(F)F)c1] | Compound 9u |
| 46 | 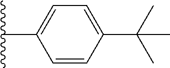 [SMILES: *c1ccc(C(C)(C)C)cc1] | Compound 9i | 57 | 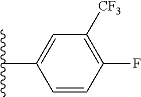 [SMILES: *c1ccc(F)c(C(F)(F)F)c1] | Compound 9v |
| 47 | 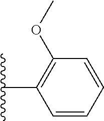 [SMILES: *c1ccccc1OC] | Compound 9j | 58 | 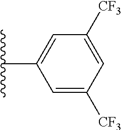 [SMILES: *c1cc(C(F)(F)F)cc(C(F)(F)F)c1] | Compound 9w |
| 48 | 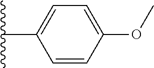 [SMILES: *c1ccc(OC)cc1] | Compound 9k | 59 | 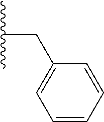 [SMILES: *Cc1ccccc1] | Compound 9y |

### Experimental Example 1

Evaluation of Anti-Tumor Cell Proliferation Activity for Target Compounds in Two Series of Examples and Comparative Examples of the present disclosure

The obtained quinazolinyl-aryl urea derivatives were screened for compounds with optimal activity using the following method. All cell lines employed in this experiment were of human origin, including: bladder cancer cells: J82, UM-UC-3, T24, 5637, pancreatic cancer cells: BxPC-3, PANC-1, osteosarcoma cells: 143B, MG-63, cervical cancer cells: HeLa, breast cancer cells: MCF-7, NSCLC cells: A549, hepatoblastoma cells: Hep G2, and ovarian cancer cells: SK-OV-3, as well as normal cell lines: immortalized ureteral epithelial cells (SV-HUC-1), embryonic lung fibroblasts (WI-38).

Experimental Procedure for Anti-Proliferative Activity Evaluation: The CCK-8 assay was employed. Cells in logarithmic growth phase were harvested, adjusted to appropriate density, and uniformly seeded into 96-well plates using a multichannel pipette (180 μL cell suspension containing complete medium per well, 5,000 cells/well). Blank control wells (containing basal medium, CCK-8 solution, and DMSO only) were set. Cells were incubated at 37° C. under 5% CO₂. After 70% confluency was reached, test compounds (target compounds/positive controls) were added at final concentrations of 1, 2, 4, 6, 8, 12, 16, 24, and 30 μM (3 repeated wells per concentration). Compounds were pre-dissolved in DMSO, sterilized, and diluted with medium before adding 20 μL/well. After co-incubation for designated durations, 10 μL CCK-8 solution was added per well followed by 1-4 h incubation (different cells have different incubation times). The reaction was terminated, and OD (optical density) values at 450 nm were measured using a microplate reader. Inhibition rates were calculated as: (OD_{control}−ODₜᵣₑₐₜₘₑₙₜ)/OD_{control}×100%. Median inhibitory concentrations (IC₅₀ values) were derived using Origin v8.0 software.

The inventor previously combined the pharmacophores “carbamido group” from Sora and “4-aminoquinazolinyl group” from Gef, initially synthesizing quinazolinyl-aryl urea derivatives I-1 to I-16 and II-1 to II-17 with benzyl linkers. These compounds bore 6-position modifications (CH₃O or H) on the quinazoline ring but exhibit suboptimal lipid and water solubility, limiting formulation to oral dosage forms and hindering injectable/intracavitary infusion preparations. In contrast, compounds 8-1a to 8-1v and 8-2a to 8-2z of the present disclosure incorporated 6-position modifications that could balance hydrophilic-hydrophobic properties, enabling injectable/intracavitary preparations. Crucially, as shown in Tables 6 to 15, with identical rightmost benzene ring substituents, compounds 8-1j, 8-2h, 8-2j, and 8-2y demonstrate superior anti-proliferative activity against UM-UC-3 cells and osteosarcoma cells (143B and MG-63).

Relative to compounds 8-1a to 8-1v and 8-2a to 8-2z, compounds 8-1 to 8-23 differ in their 6-position quinazoline ring modifications. As shown in Tables 6 to 13 and Table 16, with identical substituents on the rightmost benzene ring, compounds 8-1h, 8-1r, 8-2e, 8-2h, 8-2s, and 8-2y exhibit superior anti-proliferative activity against bladder cancer cells (T24 and 5637), A549, and Hep G2 compared to their counterparts in the 8-1 to 8-23 series. Notably for Hep G2 (Tables 12 and 16), compound 8-2e achieves an IC₅₀ of 3.68±0.19 M on Hep G2, whereas compound 8-9 (with minimum IC₅₀ in 8-1 to 8-23) shows an IC₅₀ of 12.16±0.17 μM. All compounds 8-2a to 8-2z surpass the 8-1 to 8-23 series in anti-proliferative activity against Hep G2 when with identical substituents on the rightmost benzene ring. Additionally, compounds 8-2h and 8-2j demonstrate significantly enhanced activity against A549 versus the 8-1 to 8-23 series.

Relative to compounds 8-1a to 8-1v and 8-2a to 8-2z, compounds 9a to 9y feature distinct 6-position quinazoline modifications. Per Tables 6 to 13 and Table 17, with identical substituents on the rightmost benzene ring, compounds 8-1j, 8-2e, 8-2g, 8-2j, 8-2s, and 8-2y display greater anti-proliferative activity against bladder cancer cells (T24 and 5637) and Hep G2 than their counterparts in 9a to 9y series. Critically for Hep G2 (Tables 12 and 17), compound 8-2e's IC₅₀ is 3.68±0.19 M on Hep G2, while compound 9w (with minimum IC₅₀ in 9a to 9y) shows IC₅₀=5.64±0.32 μM, indicating that the activity of 8-2e is significantly better than that of 9a to 9y.

As shown in Tables 6 to 13, bladder cancer cells (J82, UM-UC-3, T24, 5637) exhibit sensitivity to several compounds, particularly 8-1j, 8-2h, and 8-2j. For instance: IC₅₀ values of 8-1j and 8-2j against J82 cells are 2.98±0.27 M and 3.42±0.25 M, respectively; the IC₅₀ values against 5637 cells are 3.29±0.27 μM and 2.97±0.23 μM, respectively. Osteosarcoma cells (143B, MG-63) are sensitive to 8-1j, 8-2h, and 8-2z, and IC₅₀ values of the 8-1j, 8-2h, and 8-2z are less than 5 M. Among pancreatic cancer cells: BxPC-3 is sensitive to 8-1j and PANC-1 is sensitive to 8-2h. Notably, 8-2h demonstrates significantly stronger anti-proliferative activity against BxPC-3 than positive control Sora, which shows weak anti-proliferative activity.

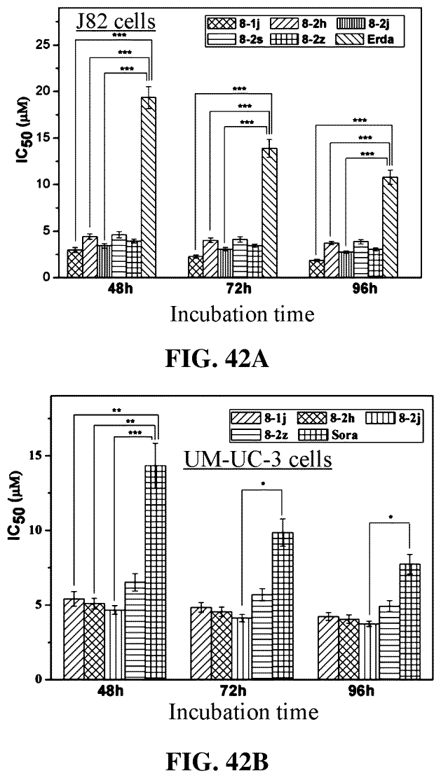

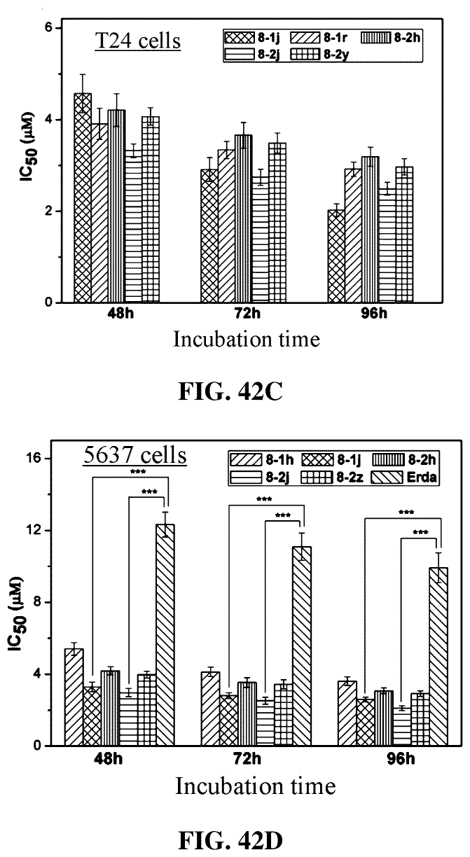

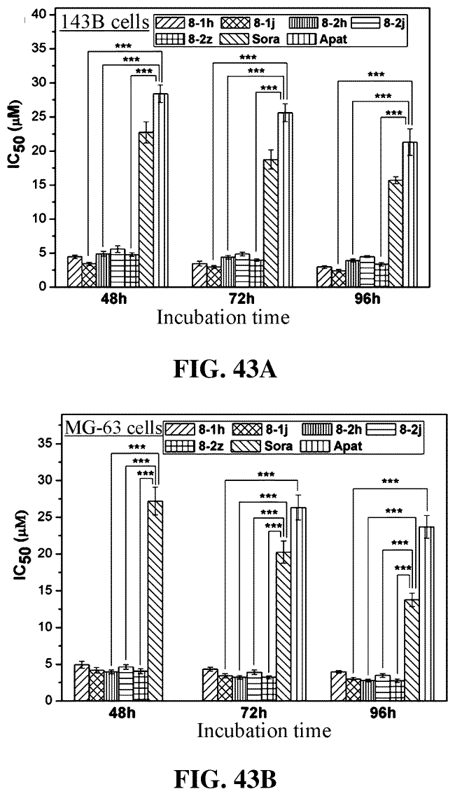

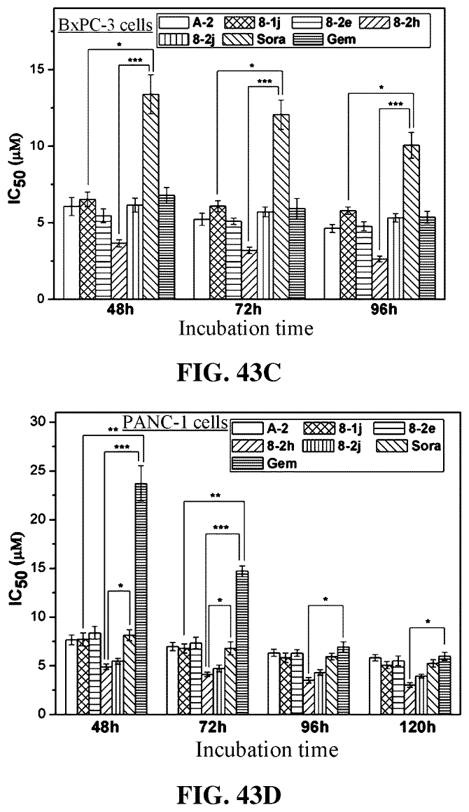

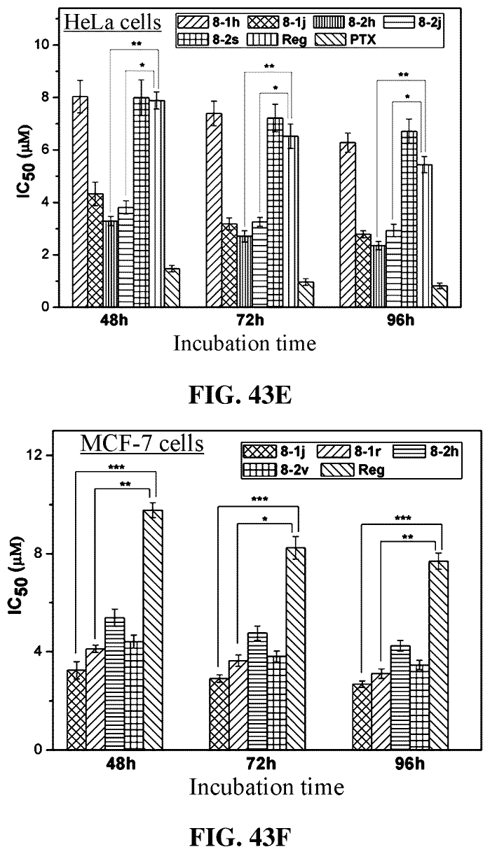

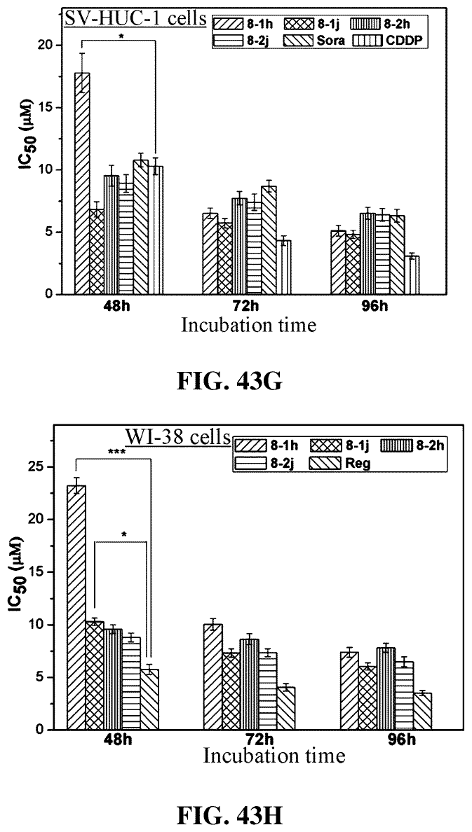

Then inventor focused on evaluating the anti-proliferative activities of the above-mentioned typical compounds after incubation with various tumor cell lines and normal cell lines for different times. The results are shown in FIGS. 42A-42D and FIGS. 43A-43H, in which *p<0.05, **p<0.01, and *** p<0.001. Key conclusions emerge: (1) Tumor cell IC₅₀ values decrease with increasing compound concentration and prolonged incubation, indicating concentration- and time-dependent effects. (2) Four compounds (8-1h, 8-1j, 8-2h, 8-2j) display potent anti-proliferative activity against multiple cell lines. Particularly, 8-1j and 8-2h outperform marketed drugs (Erda, Apat). (3) Pancreatic cancer cells (BxPC-3/PANC-1) show heightened sensitivity to Series II compounds (8-2e, 8-2h, 8-2j) versus Series I. Especially, BxPC-3 cells are very sensitive to compound 8-2h, indicating that 8-2h could surpass 8-1j, 9g (A-2), I-8, Sora, and Erlo. Based on these findings, in-depth studies of representative compounds were initiated.

TABLE 6
Anti-proliferative activity (IC₅₀ ± standard deviation; μM)^{a} of
target compounds 8-1a to 8-1v against four bladder cancer cell lines

| Compound | J82 | UM-UC-3 | T24 | 5637 |
|---|---|---|---|---|
| 8-1a | >30 | >30 | >30 | 26.39 ± 1.63 |
| 8-1c | 6.05 ± 0.63 | 14.10 ± 0.82 | 11.48 ± 0.66 | 12.97 ± 0.85 |
| 8-1d | 5.89 ± 0.57 | 13.21 ± 0.77 | 6.59 ± 0.79 | 7.72 ± 0.66 |
| 8-1g | 19.69 ± 0.98 | 26.02 ± 1.79 | 24.77 ± 0.62 | 22.49 ± 0.79 |
| 8-1h | 5.64 ± 0.39 | 8.74 ± 0.52 | 6.89 ± 0.48 | 5.41 ± 0.36 |
| 8-1i | 22.38 ± 1.82 | 10.01 ± 0.52 | 16.29 ± 0.81 | 10.32 ± 0.97 |
| 8-1j | 2.98 ± 0.27 | 3.53 ± 0.39 | 4.57 ± 0.42 | 3.29 ± 0.27 |
| 8-1l | 6.85 ± 0.57 | 16.79 ± 0.88 | 10.43 ± 0.92 | 15.79 ± 0.95 |
| 8-1m | 7.21 ± 0.66 | 11.09 ± 0.74 | 8.82 ± 0.49 | 6.06 ± 0.58 |
| 8-1o | 6.88 ± 0.49 | 5.84 ± 0.53 | 7.31 ± 0.55 | 5.34 ± 0.39 |
| 8-1q | 4.26 ± 0.31 | 6.25 ± 0.88 | 6.83 ± 0.87 | 5.78 ± 0.69 |
| 8-1r | 6.33 ± 0.61 | 6.14 ± 0.53 | 3.91 ± 0.34 | 3.59 ± 0.16 |
| 8-1s | 5.63 ± 0.54 | 6.72 ± 0.53 | 6.95 ± 0.51 | 5.93 ± 0.44 |
| 8-1v | 13.91 ± 0.95 | 18.97 ± 0.34 | 15.77 ± 0.89 | 11.21 ± 0.97 |
| Sora | −^{b} | 14.32 ± 1.51 | − | − |
| Gef | >30 | 26.12 ± 2.53 | − | − |
| Erda | 19.35 ± 1.18 | − | − | 12.32 ± 0.70 |

^{a}indicated that anti-proliferative activity was expressed as IC₅₀ values presented as IC₅₀ mean ± SD (standard deviation) (μM). Unless otherwise specified, compounds were co-incubated with cells for 48 h. IC₅₀ > 30 μM indicated weak anti-proliferative activity against the cells; this criterion applied hereafter.
Abbreviations: Sora: sorafenib; Gef: gefitinib; Erda: erdafitinib.
^{b}“—” denoted undetermined anti-proliferative activity of the compound. This notation applied hereafter.

TABLE 7
Anti-proliferative activity (IC₅₀ ± standard deviation; μM) of target
compounds 8-1a to 8-1v against 143B, MG-63, BxPC-3, and PANC-1 cells

| Compound | 143B | MG-63 | BxPC-3 | PANC-1 |
|---|---|---|---|---|
| 8-1a | 18.66 ± 1.63 | 27.33 ± 1.51 | 15.69 ± 0.78 | >30 |
| 8-1c | 12.95 ± 0.84 | 7.34 ± 0.65 | 16.51 ± 0.92 | >30 |
| 8-1d | 8.62 ± 0.53 | 7.73 ± 0.49 | 13.71 ± 0.73 | 27.39 ± 2.24 |
| 8-1g | 19.32 ± 0.74 | 10.91 ± 0.93 | 10.79 ± 0.52 | 13.09 ± 0.78 |
| 8-1h | 4.46 ± 0.24 | 4.92 ± 0.46 | 7.61 ± 0.76 | 9.63 ± 0.92 |
| 8-1i | 9.31 ± 0.97 | 11.09 ± 0.95 | 13.29 ± 1.05 | 14.37 ± 0.67 |
| 8-1j | 3.43 ± 0.25 | 4.17 ± 0.38 | 6.53 ± 0.48 | 7.72 ± 0.66 |
| 8-1l | 9.03 ± 0.61 | 7.03 ± 0.48 | 13.04 ± 0.97 | >30 |
| 8-1m | 5.62 ± 0.51 | 5.69 ± 0.65 | 7.79 ± 0.44 | 23.17 ± 2.03 |
| 8-1o | 5.99 ± 0.43 | 5.89 ± 0.47 | 7.73 ± 0.82 | >30 |
| 8-1q | 4.57 ± 0.44 | 5.81 ± 0.63 | 7.78 ± 0.53 | 19.51 ± 1.53 |
| 8-1r | 5.41 ± 0.39 | 5.31 ± 0.46 | 7.39 ± 0.62 | 11.92 ± 0.94 |
| 8-1s | 4.95 ± 0.69 | 7.17 ± 0.52 | 7.31 ± 0.76 | 12.66 ± 0.71 |
| 8-1v | 22.39 ± 1.63 | 15.57 ± 0.92 | 16.78 ± 1.25 | >30 |
| Sora | 22.73 ± 1.59 | 27.19 ± 1.89 | 13.39 ± 1.27 | 8.13 ± 0.56 |
| Apat ^{a} | 28.39 ± 1.29 | >30 | − | − |
| Gem | − | − | 6.79 ± 0.52 | 23.69 ± 1.82 |
| Erlo | − | − | 18.32 ± 1.46 | 27.39 ± 1.77 |

^{a} Apat: apatinib mesylate. Gem: gemcitabine; Erlo: erlotinib hydrochloride.

TABLE 8
Anti-proliferative activity (IC₅₀ ± standard deviation; μM) of target
compounds 8-1a to 8-1v against HeLa, MCF-7, A549, and Hep G2 cells

| Compound | HeLa | MCF-7 | A549 | Hep G2 |
|---|---|---|---|---|
| 8-1a | >30 | >30 | >30 | >30 |
| 8-1c | >30 | 25.81 ± 2.53 | 18.39 ± 0.98 | 7.27 ± 0.51 |
| 8-1d | 16.05 ± 0.71 | >30 | 15.81 ± 0.84 | 7.04 ± 0.48 |
| 8-1g | 23.02 ± 0.99 | 20.29 ± 1.88 | 26.37 ± 2.59 | 12.73 ± 0.74 |
| 8-1h | 8.03 ± 0.62 | 8.49 ± 0.88 | 12.28 ± 0.84 | 4.67 ± 0.38 |
| 8-1i | 14.07 ± 0.89 | 11.49 ± 0.76 | 15.95 ± 0.64 | 10.58 ± 0.89 |
| 8-1j | 4.33 ± 0.45 | 3.25 ± 0.35 | 4.39 ± 0.34 | 3.83 ± 0.36 |
| 8-1l | 21.29 ± 1.24 | 15.16 ± 0.93 | 21.39 ± 1.96 | 7.29 ± 0.54 |
| 8-1m | 13.79 ± 0.77 | 14.80 ± 0.86 | 14.97 ± 0.93 | 7.83 ± 0.66 |
| 8-1o | 12.39 ± 0.89 | 9.33 ± 0.71 | 11.76 ± 0.63 | 6.83 ± 0.52 |
| 8-1q | 6.32 ± 0.37 | 6.64 ± 0.52 | 10.86 ± 0.67 | 7.09 ± 0.35 |
| 8-1r | 5.76 ± 0.57 | 4.12 ± 0.15 | 8.62 ± 0.55 | 5.59 ± 0.45 |
| 8-1s | 6.81 ± 0.45 | 10.35 ± 0.68 | 12.56 ± 0.48 | 4.55 ± 0.47 |
| 8-1v | >30 | >30 | 16.52 ± 0.77 | 10.63 ± 0.94 |
| Reg ^{a} | 7.89 ± 0.33 | 9.77 ± 0.30 | 7.89 ± 0.68 | − |
| PTX | 1.48 ± 0.12 | − | − | − |

^{a} Reg: regorafenib; PTX: paclitaxel.

TABLE 9
Anti-proliferative activity (IC₅₀ ± standard
deviation; μM) of target compounds 8-1a to 8-1v
against SK-OV-3 and two normal cells (SV-HUC-1 and WI-38)

| Compound | SK-OV-3 | SV-HUC-1 | WI-38 |
|---|---|---|---|
| 8-1a | 28.11 ± 1.72 | >30 | 25.91 ± 2.74 |
| 8-1c | 9.73 ± 0.69 | 15.96 ± 0.81 | 13.91 ± 0.73 |
| 8-1d | 9.26 ± 0.87 | 12.82 ± 0.66 | 12.63 ± 0.92 |
| 8-1g | 6.38 ± 0.51 | >30 | >30 |
| 8-1h | 6.19 ± 0.44 | 17.78 ± 1.59 | 23.22 ± 0.76 |
| 8-1i | 7.68 ± 0.55 | 19.32 ± 1.39 | 20.32 ± 1.63 |
| 8-1j | 5.04 ± 0.32 | 8.03 ± 0.41 | 10.28 ± 0.36 |
| 8-1l | 7.72 ± 0.59 | 15.87 ± 0.86 | 22.79 ± 0.94 |
| 8-1m | 12.79 ± 0.42 | 12.02 ± 0.57 | 14.04 ± 0.78 |
| 8-1o | 9.34 ± 0.98 | 10.72 ± 0.51 | 9.93 ± 0.53 |
| 8-1q | 8.76 ± 0.74 | 7.61 ± 0.41 | 7.97 ± 0.63 |
| 8-1r | 8.51 ± 0.87 | 7.02 ± 0.53 | 7.89 ± 0.66 |
| 8-1s | 7.29 ± 0.66 | 7.31 ± 0.35 | 7.71 ± 0.54 |
| 8-1v | >30 | >30 | 15.39 ± 0.88 |
| Sora | 13.76 ± 0.78 | 10.79 ± 0.54 | − |
| Reg | 11.81 ± 0.92 | 11.36 ± 0.69 | 5.77 ± 0.49 |
| Gef | 7.85 ± 0.68 | 23.19 ± 1.72 | − |
| Erda | 26.69 ± 2.47 | − | − |
| CDDP ^{a} | 9.89 ± 0.97 | 10.29 ± 0.68 | − |

^{a} CDDP: cisplatin.

TABLE 10
Anti-proliferative activity (IC₅₀ ± standard deviation; μM) of target
compounds 8-2a to 8-2z against four bladder cancer cell lines

| Compound | J82 | UM-UC-3 | T24 | 5637 |
|---|---|---|---|---|
| 8-2a | >30 | 25.77 ± 1.81 | 15.75 ± 0.92 | 16.19 ± 0.77 |
| 8-2b | 6.86 ± 0.52 | 7.03 ± 0.37 | 6.96 ± 0.85 | 4.79 ± 0.43 |
| 8-2c | 7.02 ± 0.58 | 8.98 ± 0.89 | 7.29 ± 0.61 | 7.06 ± 0.57 |
| 8-2d | 13.79 ± 1.33 | 7.58 ± 0.79 | 6.62 ± 0.49 | 14.33 ± 0.89 |
| 8-2e | 5.31 ± 0.26 | 6.68 ± 0.47 | 5.57 ± 0.36 | 4.31 ± 0.42 |
| 8-2f | 12.02 ± 0.87 | 12.01 ± 0.86 | 6.01 ± 0.46 | 6.91 ± 0.67 |
| 8-2g | 5.76 ± 0.31 | 11.59 ± 0.62 | 7.04 ± 0.55 | 6.04 ± 0.43 |
| 8-2h | 4.42 ± 0.29 | 5.09 ± 0.35 | 4.21 ± 0.36 | 4.18 ± 0.22 |
| 8-2i | 5.88 ± 0.35 | 6.32 ± 0.44 | 5.92 ± 0.47 | 4.24 ± 0.32 |
| 8-2j | 3.42 ± 0.25 | 4.66 ± 0.29 | 3.32 ± 0.15 | 2.97 ± 0.23 |
| 8-2k | 5.68 ± 0.42 | 7.19 ± 0.52 | 5.34 ± 0.27 | 5.63 ± 0.38 |
| 8-2l | 7.83 ± 0.69 | 12.04 ± 0.94 | 6.02 ± 0.46 | 9.12 ± 0.59 |
| 8-2n | 14.98 ± 1.19 | 12.79 ± 0.76 | 5.82 ± 0.36 | 8.61 ± 0.73 |
| 8-2o | 16.71 ± 1.52 | 15.86 ± 0.95 | 7.93 ± 0.22 | 12.41 ± 0.89 |
| 8-2q | 6.69 ± 0.53 | 7.19 ± 0.47 | 6.76 ± 0.38 | 4.32 ± 0.33 |
| 8-2r | 4.73 ± 0.38 | 11.67 ± 0.96 | 5.12 ± 0.39 | 6.29 ± 0.37 |
| 8-2s | 4.62 ± 0.33 | 10.02 ± 0.73 | 7.73 ± 0.47 | 4.87 ± 0.29 |
| 8-2t | 5.46 ± 0.41 | 6.79 ± 0.41 | 6.47 ± 0.25 | 7.32 ± 0.57 |
| 8-2u | 12.94 ± 0.66 | 11.92 ± 0.66 | 14.12 ± 0.77 | 13.19 ± 0.78 |
| 8-2v | 11.07 ± 0.69 | 6.77 ± 0.69 | 4.73 ± 0.42 | 11.79 ± 0.95 |
| 8-2w | 7.84 ± 0.52 | 6.42 ± 0.16 | 5.92 ± 0.54 | 13.08 ± 0.67 |
| 8-2y | 4.13 ± 0.32 | 3.92 ± 0.24 | 4.07 ± 0.19 | 4.92 ± 0.31 |
| 8-2z | 3.93 ± 0.22 | 3.71 ± 0.19 | 5.33 ± 0.52 | 3.96 ± 0.19 |

TABLE 11
Anti-proliferative activity (IC₅₀ ± standard deviation; μM) of target
compounds 8-2a to 8-2z against 143B, MG-63, BxPC-3, and PANC-1 cells

| Compound | 143B | MG-63 | BxPC-3 | PANC-1 |
|---|---|---|---|---|
| 8-2a | >30 | 17.36 ± 1.38 | >30 | >30 |
| 8-2b | 6.47 ± 0.34 | 6.44 ± 0.81 | 5.92 ± 0.38 | 14.52 ± 0.94 |
| 8-2c | 6.01 ± 0.46 | 6.83 ± 0.62 | 13.38 ± 0.88 | 19.23 ± 1.08 |
| 8-2d | 6.92 ± 0.39 | 7.29 ± 0.62 | 9.29 ± 0.73 | 25.39 ± 2.04 |
| 8-2e | 5.19 ± 0.33 | 7.03 ± 0.71 | 5.46 ± 0.44 | 8.37 ± 0.66 |
| 8-2f | 5.75 ± 0.36 | 6.92 ± 0.53 | 9.06 ± 0.42 | 12.75 ± 0.62 |
| 8-2g | 5.37 ± 0.29 | 6.04 ± 0.65 | 8.56 ± 0.72 | 8.79 ± 0.98 |
| 8-2h | 4.91 ± 0.36 | 3.97 ± 0.26 | 3.66 ± 0.24 | 4.88 ± 0.29 |
| 8-2i | 6.38 ± 0.33 | 4.79 ± 0.46 | 8.97 ± 0.89 | 9.42 ± 0.83 |
| 8-2j | 5.59 ± 0.49 | 4.64 ± 0.32 | 6.15 ± 0.47 | 5.46 ± 0.31 |
| 8-2k | 6.08 ± 0.37 | 6.38 ± 0.43 | 7.11 ± 0.79 | 15.87 ± 0.94 |
| 8-2l | 6.75 ± 0.41 | 7.69 ± 0.58 | 18.86 ± 1.24 | >30 |
| 8-2n | 6.71 ± 0.48 | 8.39 ± 0.66 | 14.38 ± 1.55 | 18.79 ± 1.43 |
| 8-2o | 13.77 ± 0.82 | 9.92 ± 0.82 | 22.39 ± 1.27 | >30 |
| 8-2q | 7.08 ± 0.85 | 8.61 ± 0.53 | 13.19 ± 0.78 | 15.19 ± 0.67 |
| 8-2r | 8.91 ± 0.77 | 7.02 ± 0.58 | 10.26 ± 0.93 | 14.63 ± 0.78 |
| 8-2s | 5.94 ± 0.43 | 10.98 ± 0.97 | 9.72 ± 0.95 | 10.37 ± 0.62 |
| 8-2t | 9.42 ± 0.56 | 11.74 ± 0.92 | 11.34 ± 0.73 | 15.58 ± 0.93 |
| 8-2u | 13.63 ± 0.92 | 16.02 ± 1.07 | 25.42 ± 1.57 | >30 |
| 8-2v | 6.84 ± 0.59 | 8.02 ± 0.79 | 14.88 ± 1.28 | 14.89 ± 0.79 |
| 8-2w | 8.04 ± 0.45 | 8.33 ± 0.81 | 15.54 ± 0.92 | 15.12 ± 0.66 |
| 8-2y | 6.74 ± 0.52 | 5.91 ± 0.46 | 10.96 ± 0.85 | 7.36 ± 0.42 |
| 8-2z | 4.77 ± 0.26 | 4.05 ± 0.34 | 10.31 ± 0.93 | 14.91 ± 0.98 |

TABLE 12
Anti-proliferative activity (IC₅₀ ± standard deviation; μM) of target
compounds 8-2a to 8-2z against HeLa, MCF-7, Hep G2, and SK-OV-3 cells

| Compound | HeLa | MCF-7 | Hep G2 | SK-OV-3 |
|---|---|---|---|---|
| 8-2a | >30 | 26.29 ± 1.46 | 25.72 ± 1.81 | 22.38 ± 1.33 |
| 8-2b | 5.72 ± 0.41 | 7.25 ± 0.66 | 4.04 ± 0.28 | 6.21 ± 0.55 |
| 8-2c | 6.19 ± 0.53 | 6.69 ± 0.48 | 4.69 ± 0.36 | 7.94 ± 0.66 |
| 8-2d | 4.79 ± 0.38 | 7.92 ± 0.51 | 3.82 ± 0.25 | 6.79 ± 0.62 |
| 8-2e | 5.26 ± 0.29 | 7.65 ± 0.69 | 3.68 ± 0.19 | 7.26 ± 0.72 |
| 8-2f | 7.25 ± 0.38 | 8.09 ± 0.48 | 11.71 ± 0.84 | 14.19 ± 0.84 |
| 8-2g | 5.62 ± 0.44 | 7.62 ± 0.77 | 6.38 ± 0.44 | 8.96 ± 0.83 |
| 8-2h | 3.29 ± 0.18 | 5.38 ± 0.35 | 4.66 ± 0.38 | 6.12 ± 0.55 |
| 8-2i | 4.97 ± 0.48 | 6.21 ± 0.63 | 3.85 ± 0.29 | 6.69 ± 0.49 |
| 8-2j | 3.81 ± 0.25 | 5.73 ± 0.59 | 3.81 ± 0.24 | 4.61 ± 0.38 |
| 8-2k | 5.51 ± 0.49 | 6.68 ± 0.79 | 4.99 ± 0.47 | 6.32 ± 0.56 |
| 8-2l | 6.42 ± 0.69 | 7.29 ± 0.63 | 4.74 ± 0.62 | 11.71 ± 0.88 |
| 8-2n | 12.69 ± 0.92 | 12.76 ± 0.85 | 8.51 ± 0.67 | 5.93 ± 0.61 |
| 8-2o | 13.71 ± 1.29 | 16.19 ± 1.07 | 16.02 ± 0.96 | 11.92 ± 0.94 |
| 8-2q | 7.72 ± 0.34 | 6.44 ± 0.71 | 10.66 ± 0.98 | 5.62 ± 0.47 |
| 8-2r | 7.37 ± 0. 53 | 11.42 ± 0.76 | 7.02 ± 0.49 | 11.32 ± 0.83 |
| 8-2s | 7.99 ± 0.68 | 5.88 ± 0.57 | 4.91 ± 0.55 | 6.02 ± 0.39 |
| 8-2t | 7.29 ± 0.24 | 5.94 ± 0.36 | 4.76 ± 0.45 | 13.29 ± 0.84 |
| 8-2u | >30 | 9.92 ± 0.86 | 14.26 ± 1.33 | 15.61 ± 0.92 |
| 8-2v | 6.98 ± 0.83 | 4.41 ± 0.27 | 4.97 ± 0.35 | 5.49 ± 0.63 |
| 8-2w | 6.42 ± 0.56 | 5.59 ± 0.46 | 5.87 ± 0.49 | 5.16 ± 0.27 |
| 8-2y | 5.02 ± 0.49 | 5.71 ± 0.54 | 5.92 ± 0.66 | 6.23 ± 0.56 |
| 8-2z | 6.39 ± 0.68 | 6.32 ± 0.49 | 5.23 ± 0.52 | 8.69 ± 0.72 |

TABLE 13
Anti-proliferative activity (IC₅₀ ± standard deviation; μM) of target
compounds 8-2a to 8-2z against A549, SV-HUC-1, and WI-38 cells

| Compound | A549 | SV-HUC-1 | WI-38 |
|---|---|---|---|
| 8-2a | >30 | 26.39 ± 1.69 | 17.59 ± 0.72 |
| 8-2b | 5.47 ± 0.22 | 8.74 ± 0.63 | 9.02 ± 0.41 |
| 8-2c | 8.39 ± 0.85 | 8.28 ± 0.51 | 11.23 ± 0.49 |
| 8-2d | 9.23 ± 0.61 | 7.94 ± 0.44 | 9.79 ± 0.38 |
| 8-2e | 4.92 ± 0.29 | 7.56 ± 0.38 | 10.87 ± 0.66 |
| 8-2f | 7.67 ± 0.93 | 11.62 ± 0.79 | 9.89 ± 0.53 |
| 8-2g | 6.59 ± 0.64 | 7.42 ± 0.35 | 9.39 ± 0.41 |
| 8-2h | 3.41 ± 0.26 | 9.53 ± 0.83 | 9.58 ± 0.43 |
| 8-2i | 7.63 ± 0.87 | 10.79 ± 0.74 | 10.53 ± 0.36 |
| 8-2j | 3.09 ± 0.24 | 8.91 ± 0.72 | 8.79 ± 0.42 |
| 8-2k | 14.22 ± 1.31 | 13.03 ± 0.88 | 11.17 ± 0.65 |
| 8-2l | >30 | 10.86 ± 0.69 | 13.86 ± 0.71 |
| 8-2n | 15.39 ± 0.83 | 14.38 ± 0.77 | 9.98 ± 0.53 |
| 8-2o | 27.19 ± 2.04 | 16.19 ± 0.84 | 12.68 ± 0.89 |
| 8-2q | 13.31 ± 0.86 | 9.49 ± 0.73 | 7.36 ± 0.23 |
| 8-2r | 12.29 ± 0.92 | 8.41 ± 0.43 | 7.72 ± 0.39 |
| 8-2s | 7.32 ± 0.85 | 8.36 ± 1.62 | 11.51 ± 0.62 |
| 8-2t | 11.76 ± 0.88 | 8.92 ± 0.49 | 10.58 ± 0.64 |
| 8-2u | 12.39 ± 1.43 | 23.12 ± 0.97 | 15.92 ± 0.85 |
| 8-2v | 7.98 ± 0.92 | 7.33 ± 0.46 | 8.71 ± 0.52 |
| 8-2w | 9.69 ± 0.72 | 7.19 ± 0.68 | 9.49 ± 0.42 |
| 8-2y | 7.01 ± 0.62 | 8.96 ± 0.67 | 9.89 ± 0.23 |
| 8-2z | 11.12 ± 0.83 | 8.02 ± 0.81 | 10.93 ± 0.59 |

TABLE 14
Anti-proliferative activity (IC₅₀ ± standard
deviation; μM) of target compounds I-1 to I-16
and II-1 to II-17 against UM-UC-3, 143B, and MG-63 cells

| Compound | UM-UC-3 | 143B | MG-63 |
|---|---|---|---|
| I-1 | >30 | 18.29 ± 1.74 | 27.12 ± 1.45 |
| I-2 | 11.21 ± 0.85 | 6.34 ± 0.56 | 11.24 ± 0.67 |
| I-3 | 9.02 ± 0.74 | 5.94 ± 0.33 | 12.05 ± 0.76 |
| I-5 | 10.53 ± 0.71 | 6.47 ± 0.28 | 13.38 ± 0.95 |
| I-6 | 20.59 ± 2.37 | 19.29 ± 1.67 | 25.39 ± 1.74 |
| I-7 | 18.62 ± 1.35 | 21.33 ± 1.59 | >30 |
| I-8 | 7.91 ± 0.59 | 6.51 ± 0.69 | 6.02 ± 0.44 |
| I-9 | 9.56 ± 0.72 | 6.11 ± 0.42 | 14.75 ± 1.22 |
| I-10 | 8.77 ± 0.41 | 7.74 ± 0.39 | 11.09 ± 0.54 |
| I-14 | >30 | >30 | >30 |
| II-1 | >30 | >30 | >30 |
| II-2 | 22.79 ± 1.38 | 13.24 ± 0.86 | 19.28 ± 1.76 |
| II-3 | 17.82 ± 0.57 | 11.29 ± 0.88 | 17.84 ± 0.95 |
| II-5 | 8.72 ± 0.36 | 7.26 ± 0.35 | 10.46 ± 0.82 |
| II-9 | 9.77 ± 0.52 | 10.48 ± 0.43 | 9.24 ± 0.71 |
| II-13 | 9.63 ± 0.52 | 7.56 ± 0.27 | 20.82 ± 0.95 |
| II-17 | 7.69 ± 0.56 | 10.34 ± 0.67 | 8.54 ± 0.59 |

TABLE 15
Anti-proliferative activity (IC₅₀ ± standard
deviation; μM) of target compounds I-1 to I-16
and II-1 to II-17 against BxPC-3, PANC-1, and SK-OV-3 cells

| Compound | BxPC-3 | PANC-1 | SK-OV-3 |
|---|---|---|---|
| I-1 | 18.58 ± 1.66 | >30 | 28.39 ± 1.46 |
| I-2 | 10.29 ± 0.59 | 24.39 ± 1.69 | 10.13 ± 0.69 |
| I-3 | 7.78 ± 0.69 | 17.58 ± 1.48 | 9.17 ± 0.58 |
| I-5 | 6.54 ± 0.61 | 12.46 ± 0.97 | 7.76 ± 0.38 |
| I-6 | 23.56 ± 1.47 | 26.48 ± 2.36 | 9.12 ± 0.44 |
| I-7 | 22.79 ± 1.71 | >30 | 13.73 ± 0.85 |
| 1-8 | 7.04 ± 0.33 | 9.38 ± 0.85 | 5.89 ± 0.54 |
| I-9 | 8.62 ± 0.86 | 10.73 ± 0.95 | 10.04 ± 0.53 |
| I-10 | 9.85 ± 0.76 | 11.66 ± 0.82 | 11.18 ± 0.92 |
| I-14 | 12.33 ± 0.56 | >30 | 19.71 ± 0.73 |
| II-1 | 27.62 ± 1.91 | >30 | >30 |
| II-2 | 15.49 ± 1.71 | >30 | 19.52 ± 1.37 |
| II-3 | 14.33 ± 0.86 | >30 | 14.74 ± 0.98 |
| II-5 | 9.92 ± 0.41 | 14.75 ± 1.24 | 8.40 ± 0.53 |
| II-9 | 13.26 ± 0.87 | 15.69 ± 1.52 | 7.78 ± 0.69 |
| II-13 | 8.43 ± 0.39 | 26.15 ± 2.26 | 14.97 ± 1.74 |
| II-17 | 14.19 ± 0.82 | 20.72 ± 0.96 | 9.27 ± 0.55 |

TABLE 16
Anti-proliferative activity (IC₅₀ ± standard deviation; μM) of target
compounds 8-1 to 8-23 against T24, 5637, MCF-7, A549, and Hep G2 cells

| Compound | T24 | 5637 | MCF-7 | A549 | Hep G2 |
|---|---|---|---|---|---|
| 8-1 | 18.82 ± 0.49 | 26.18 ± 0.98 | 18.30 ± 0.53 | >30 | 28.25 ± 0.49 |
| 8-2 | >30 | 25.86 ± 1.32 | 16.52 ± 0.64 | >30 | >30 |
| 8-3 | 20.61 ± 0.68 | >30 | 23.15 ± 0.35 | 24.06 ± 0.95 | >30 |
| 8-4 | 27.12 ± 0.33 | 13.95 ± 0.66 | 18.52 ± 0.63 | 22.23 ± 1.15 | >30 |
| 8-5 | 23.15 ± 0.54 | 21.66 ± 0.51 | 21.31 ± 0.42 | 21.04 ± 0.87 | >30 |
| 8-6 | 19.44 ± 0.44 | 27.52 ± 0.94 | >30 | >30 | >30 |
| 8-7 | 18.64 ± 0.53 | 13.94 ± 0.68 | >30 | >30 | 24.20 ± 0.53 |
| 8-8 | 7.73 ± 0.23 | 7.60 ± 0.18 | 10.04 ± 0.19 | 18.27 ± 0.29 | 17.71 ± 0.46 |
| 8-9 | 4.54 ± 0.04 | 6.25 ± 0.06 | 6.55 ± 0.13 | 12.46 ± 0.37 | 12.16 ± 0.17 |
| 8-10 | 18.73 ± 0.38 | 16.95 ± 0.64 | 19.27 ± 0.56 | >30 | 28.84 ± 0.58 |
| 8-11 | 23.26 ± 1.82 | 17.91 ± 0.33 | 18.28 ± 0.43 | >30 | >30 |
| 8-13 | 19.47 ± 0.27 | 13.90 ± 0.43 | 21.51 ± 0.47 | >30 | >30 |
| 8-14 | 23.11 ± 0.73 | 28.91 ± 0.53 | 18.60 ± 0.35 | 20.17 ± 0.92 | >30 |
| 8-15 | 19.07 ± 0.53 | 13.97 ± 0.72 | 16.79 ± 0.32 | 13.78 ± 0.35 | >30 |
| 8-16 | >30 | >30 | 15.93 ± 0.62 | >50 | >30 |
| 8-18 | 19.57 ± 0.45 | 13.13 ± 0.24 | 13.17 ± 0.39 | 18.80 ± 0.59 | >30 |
| 8-19 | 26.21 ± 0.94 | 17.91 ± 0.45 | 22.43 ± 0.82 | 16.09 ± 0.71 | >30 |
| 8-20 | 18.37 ± 0.81 | 14.10 ± 0.29 | 20.52 ± 0.38 | 22.38 ± 1.01 | >30 |
| 8-21 | 5.42 ± 0.05 | 6.61 ± 0.07 | 12.19 ± 0.32 | 11.93 ± 0.23 | 14.06 ± 0.20 |
| 8-23 | >30 | 26.79 ± 0.63 | 23.10 ± 0.92 | >30 | >30 |

TABLE 17
Anti-proliferative activity (IC₅₀ ± standard deviation; μM) of target
compounds 9a to 9z against T24, 5637, MCF-7, and Hep G2 cells

| Compound | T24 | 5637 | MCF-7 | Hep G2 |
|---|---|---|---|---|
| 9a | 26.69 ± 1.88 | >30 | 12.00 ± 0.92 | >30 |
| 9b | 22.33 ± 1.77 | 14.54 ± 1.04 | 11.01 ± 0.74 | 22.53 ± 1.75 |
| 9c | >30 | 14.51 ± 1.57 | 11.15 ± 0.88 | >30 |
| 9d | 29.14 ± 2.26 | 10.09 ± 0.73 | 25.94 ± 2.33 | 17.44 ± 1.75 |
| 9e | >30 | 19.62 ± 1.67 | 14.31 ± 1.07 | >30 |
| 9f | 23.71 ± 1.71 | 14.04 ± 1.21 | 19.73 ± 1.52 | >30 |
| 9g | 2.51 ± 0.33 | 1.8 ± 0.22 | 4.27 ± 0.36 | 19.12 ± 1.96 |
| 9h | 19.67 ± 1.38 | >30 | 7.31 ± 0.65 | >30 |
| 9i | 10.01 ± 0.92 | 15.25 ± 1.15 | 6.06 ± 0.57 | 28.98 ± 2.13 |
| 9j | 23.51 ± 1.61 | 15.97 ± 1.21 | >30 | 29.44 ± 2.44 |
| 9k | >30 | 17.26 ± 1.85 | >30 | >30 |
| 9m | >30 | >30 | >30 | >30 |
| 9n | 19.56 ± 1.44 | 14.00 ± 1.27 | >30 | >30 |
| 9o | 23.40 ± 2.11 | 20.89 ± 1.65 | 26.79 ± 1.78 | >30 |
| 9p | 18.14 ± 1.46 | 12.63 ± 0.87 | 16.69 ± 0.92 | >30 |
| 9q | 18.25 ± 1.55 | 15.35 ± 1.33 | 12.29 ± 0.83 | >30 |
| 9r | 13.38 ± 1.03 | 9.35 ± 0.68 | 6.56 ± 0.44 | >30 |
| 9t | 9.71 ± 0.69 | 12.63 ± 0.97 | 6.11 ± 0.25 | 12.97 ± 1.09 |
| 9u | 6.49 ± 0.49 | 13.34 ± 1.18 | 5.90 ± 0.48 | 14.76 ± 1.19 |
| 9v | 9.53 ± 0.15 | 16.32 ± 1.21 | 6.61 ± 0.39 | 14.25 ± 0.96 |
| 9w | 3.85 ± 0.47 | 10.11 ± 0.69 | 5.55 ± 0.37 | 5.64 ± 0.32 |
| 9y | 25.65 ± 1.90 | 15.64 ± 1.09 | 11.36 ± 0.73 | >30 |

### Experimental Example 2

### Effects of Compounds 8-1j, 8-2h, and 8-2j on Cell Cycle

Tumor cells (T24, UM-UC-3, 143B, BxPC-3) in logarithmic growth phase were inoculated into 70 mm culture dishes. After 70% confluency was reached, medium was replaced and treated with: compound 8-1j, ferroptosis inducer Erastin, or cell cycle blocker paclitaxel (PTX) for a specified duration. Post-incubation, cells were trypsinized, collected, centrifuged, and washed 2 times with pre-cooled phosphate-buffered saline (PBS). Pre-cooled 75% ethanol was added dropwise during vortexing for fixation at −20° C. overnight. After centrifugation (10 min), supernatant was discarded. Cells were rewashed with PBS and centrifuged. Following kit instructions (Beyotime, C1052, China), 0.5 mL staining buffer, 25 μL PI (propidium iodide) stain (20×), and 10 μL RNase A (50×) were mixed per sample. Cells were resuspended in PI staining solution and incubated at 37° C. in dark for 30 min (gentle shaking every 10 min). Samples were filtered through 200-mesh filters, transferred into a flow tube, and analyzed via BD FACSVerse flow cytometer. All experiments were conducted in 3 repeats. Compounds 8-2h and 8-2j were evaluated identically.

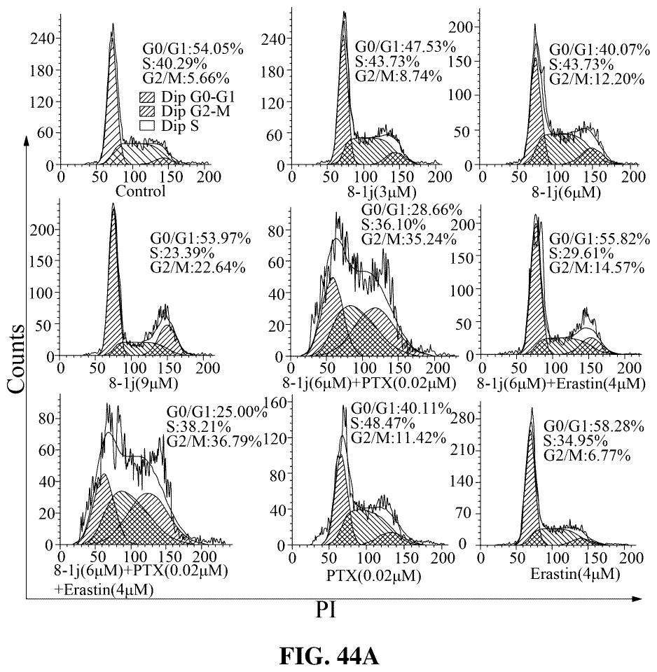

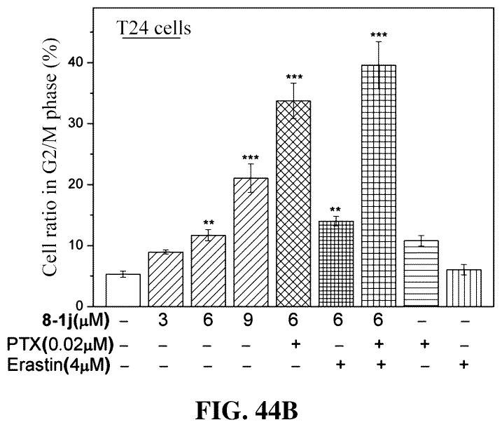

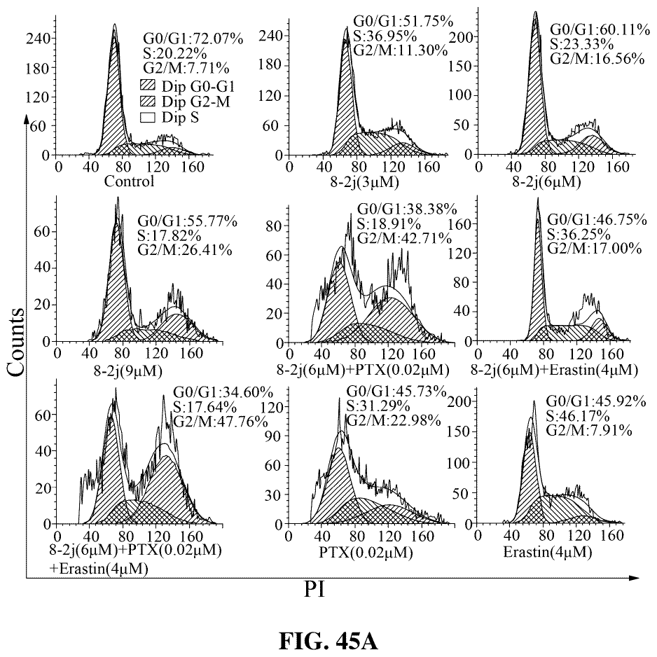

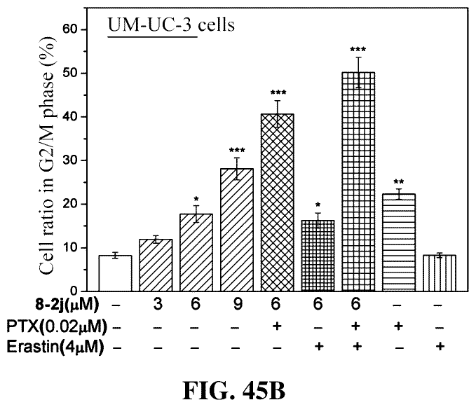

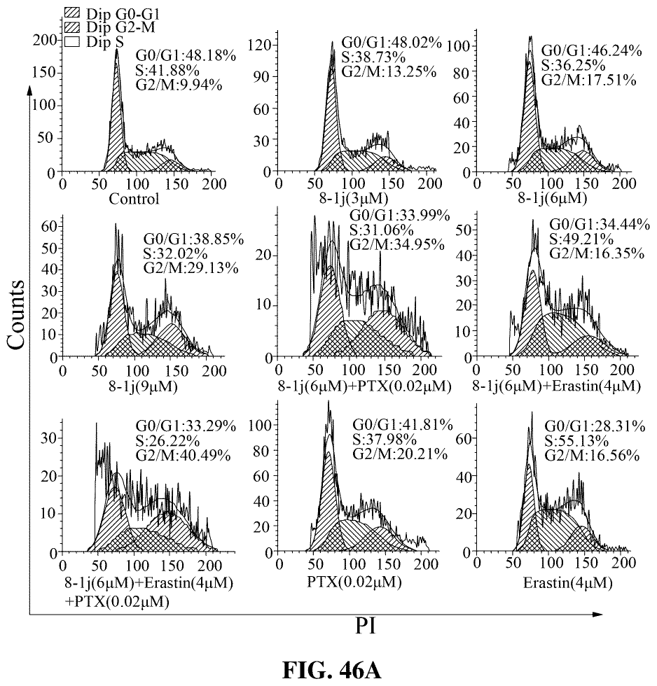

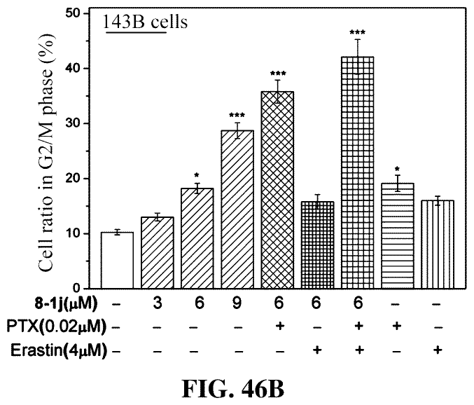

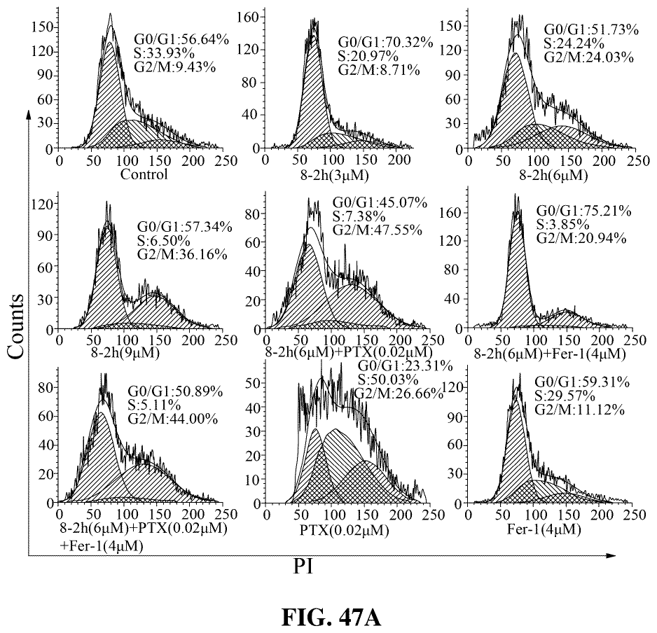

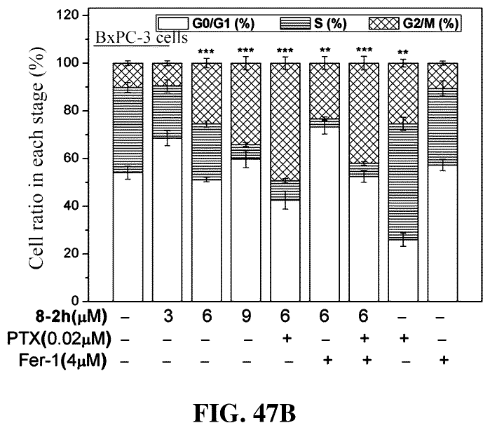

Results are shown in FIG. 44A to FIG. 47B. Asterisks denote G2/M phase cell ratio vs. blank control: *p<0.05, **p<0.01, **p<0.001. Key findings: compounds 8-1j and 8-2j arrest cell cycle at G2/M phase. Compound 8-2h exhibits concentration-dependent effects: low concentration (e.g., 3 μM): G0/G1 arrest in pancreatic cancer cells BxPC-3; high concentration (>6 μM): G2/M arrest. Synergistic effects are observed when compound 8-1j (or 8-2h or 8-2j) was co-administered with PTX (specific cell cycle blocker to G2/M). Thus, these compounds inhibit proliferation of bladder cancer, osteosarcoma, and pancreatic cancer cells via cell cycle arrest.

### Experimental Example 3

### Effects of Compound 8-1j on Cell Cycle and Ferroptosis-Related Protein Expression

Experimental method: bladder cancer T24 cells in logarithmic growth phase were inoculated into 100 mm culture dishes. After 70% confluency was reached, medium was replaced and treated with compound 8-1j or ferroptosis inhibitor Fer-1. Post-co-incubation, cells were trypsinized, collected, and washed with pre-cooled PBS. 0.2 mL of RIPA cell lysis buffer was added to each tube, mixed thoroughly, and transferred to a 1.5 mL centrifuge tube, and lysed on ice with agitation for 30 min. Lysates were centrifuged at 12,000 rpm (4° C., 10 min) to collect supernatants. Supernatants were mixed with 5×SDS-PAGE protein loading buffer, heated at 95° C. for 7 min, and stored at −80° C. SDS-PAGE (sodium dodecyl sulfate-polyacrylamide gel electrophoresis) was conducted at 80 V until entry into separating gel, then at 120 V until bromophenol blue reached the gel bottom. Gels were cut according to molecular weight indicated by Marker, transferred to membranes, blocked, and then immune reaction was performed. Protein bands were visualized by an enhanced chemiluminescence (ECL) detection system. Band densities were analyzed using ImageJ software. Three repeated experiments were conducted.

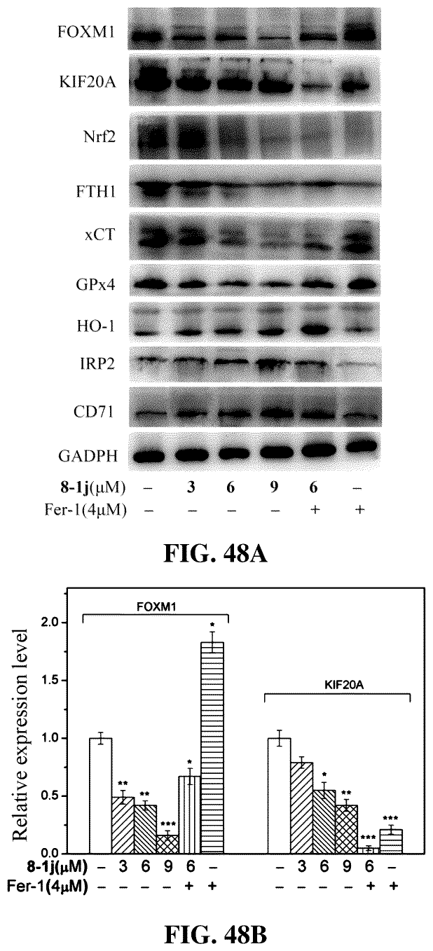

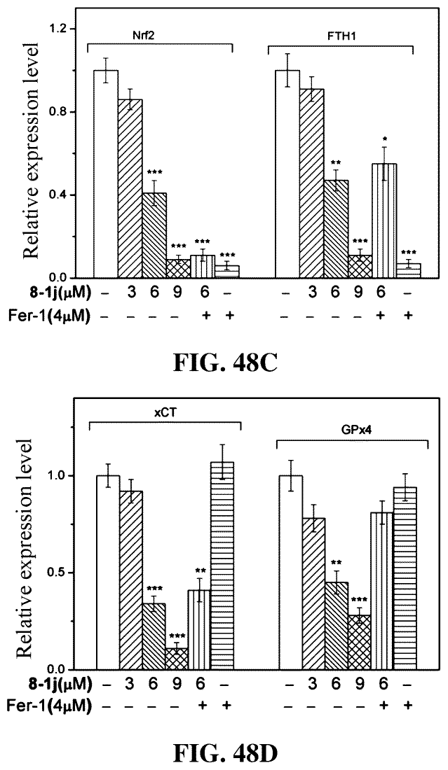

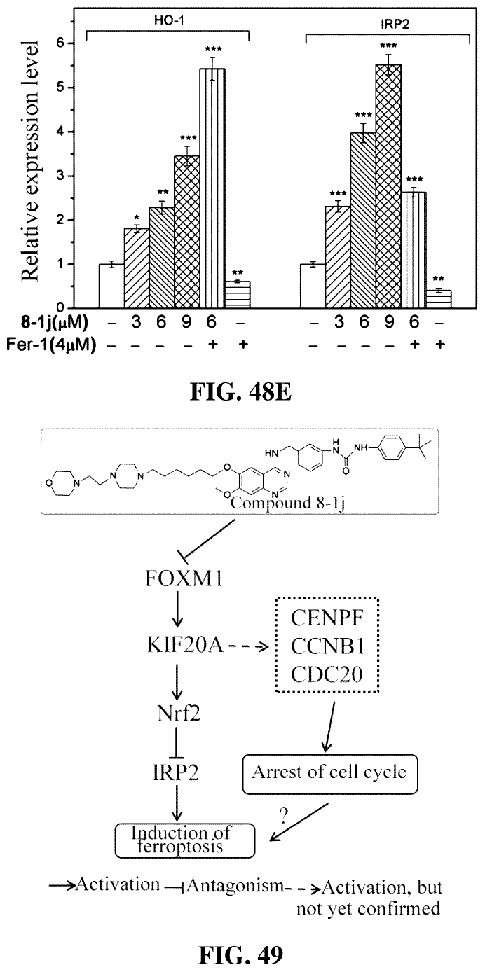

Results are shown in FIGS. 48A-48E. Asterisks denote vs. blank control: *p<0.05, **p<0.01, **p<0.001. Compound 8-1j significantly downregulates FoxM1, KIF20A, Nrf2, xCT, GPx4, and FTH1 expression in bladder cancer T24 cells concentration-dependently, while upregulating ferroptosis-related proteins HO-1, IRP2, and CD71. These results indicate that compound 8-1j could modulate cell cycle-related proteins in bladder cancer cells, suppress Nrf2/FTH1/xCT/GPx4 expression, and activate iron-regulatory protein 2 (IRP2).

The cell cycle serves as the foundation for normal cellular growth and division and represents a critical mechanism in cancer pathogenesis. In tumor cells, dysregulated cell cycle control leads to accumulation of DNA damage and uncontrolled proliferation, presenting as rapid proliferation. Kinesin family member 20A (KIF20A), a key regulator of spindle assembly during mitosis. KIF20A is overexpressed in multiple malignancies including: bladder cancer (Meng X, et al. Biol Proced Online. 2022; 24(1): 12), osteosarcoma (Li J, et al. Front Immunol. 2024; 15: 1445555), pancreatic cancer (Taniuchi K, et al. Neoplasia. 2014; 16(12): 1082-1093), prostate cancer (Yu H, et al. Cancer Cell Int. 2020; 20(1): 545), lung cancer (He H, et al. Front Pharmacol. 2022; 13: 1007429), liver cancer (Zhang C, et al. Apoptosis. 2024; 29(1-2): 103-120), breast cancer (Li T F, et al. Cancer Cell Int. 2020; 20: 123; Khongkow P, et al. Oncogene. 2016; 35(8): 990-1002), and melanoma (Jiang J, et al. Front Oncol. 2021; 11: 621430). In bladder cancer tissues, elevated KIF20A expression correlates with advanced pathological stage and poorer prognosis. Knocking down or silencing KIF20A suppresses proliferation and invasion of bladder cancer cells T24, 5637, and UM-UC-3 cells by suppressing the expression level of matrix metalloproteinase 2 (MMP-2) and matrix metalloproteinase (MMP-9). Upregulated KIF20A expression level enhances cancer cell viability (Zhang W, et al. Mol Biotechnol. 2022; 64(11): 1259-1269). High expression of KIF20A promotes the proliferation of various lung adenocarcinoma cells such as A549, and silencing KIF20A resensitizes cells to Gem (He H, et al. Front Pharmacol. 2022; 13: 1007429). Conclusion: the significant suppression of KIF20A expression by compound 8-1j in bladder cancer T24 cells, coupled with its induction of G2/M phase arrest, validate its mechanistic action against bladder cancer.

Ferroptosis requires iron ions, where excess intracellular Fe²⁺ generates reactive oxygen species (ROS) via the Fenton reaction. ROS attack polyunsaturated fatty acids (PUFAs) in biomembranes, elevating lipid peroxides and disrupting redox homeostasis, ultimately inducing cell death. Key ferroptosis-related targets include: xCT, GPx4, Nrf2, FTH1, HO-1, IRP2, and CD71. The cystine/glutamate antiporter (System Xc⁻) in biomembranes includes light chain subunit solute carrier family 7 member 11 (SLC7A11, also known as xCT) and heavy chain subunit solute carrier family 3 member 2 (SLC3A2, also known as CD98/4F2hc). Glutathione peroxidase 4 (GPx4) scavenges lipid peroxides in membranes. Sora and Erastin induce ferroptosis by inhibiting System Xc⁻ activity, depleting glutathione (GSH). Ferrostatin-1 (Fer-1) suppresses ferroptosis by inhibiting lipid peroxidation, iron accumulation and release, and oxidative stress. The Nrf2/HO-1 pathway plays a key role in protecting cells from oxidative stress. Nuclear factor erythroid 2-related factor 2 (Nrf2) regulates antioxidant responses and inhibits ferroptosis. Heme oxygenase 1 (HO-1), an ER-anchored enzyme, catalyzes heme degradation into ferrous ions, CO, and biliverdin. Increasing evidence indicates elevated HO-1 acts as an important positive regulator in the process of ferroptosis. Upregulated HO-1 expression leads to heme degradation and intracellular iron overload, triggering lipid peroxidation and promoting ferroptosis. Iron-regulatory protein 2 (TRP2/IREB2) modulates cellular iron metabolism.

Thus, compound 8-1j demonstrates dual mechanisms, cell cycle arrest and potential ferroptosis induction, with the proposed mechanism illustrated in FIG. 49.

### Experimental Example 4

### Activity Evaluation of Compounds 8-1j and 8-2h in Several Tumor-Bearing Nude Mouse Models

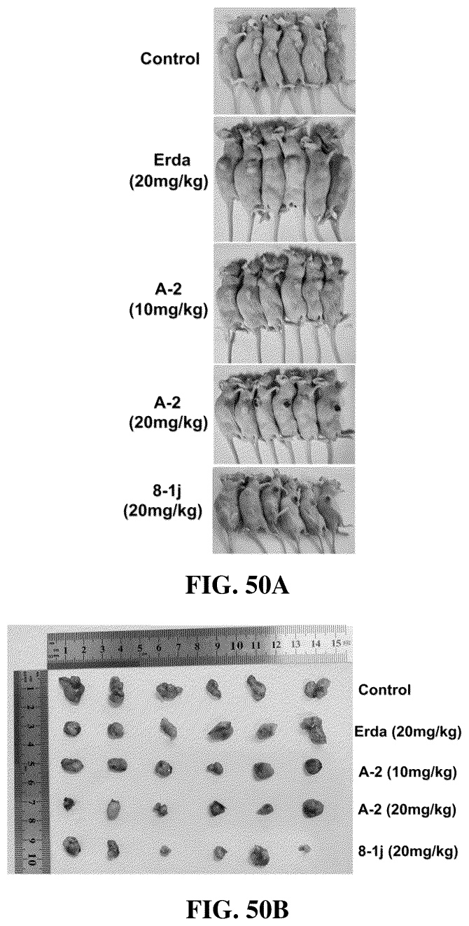

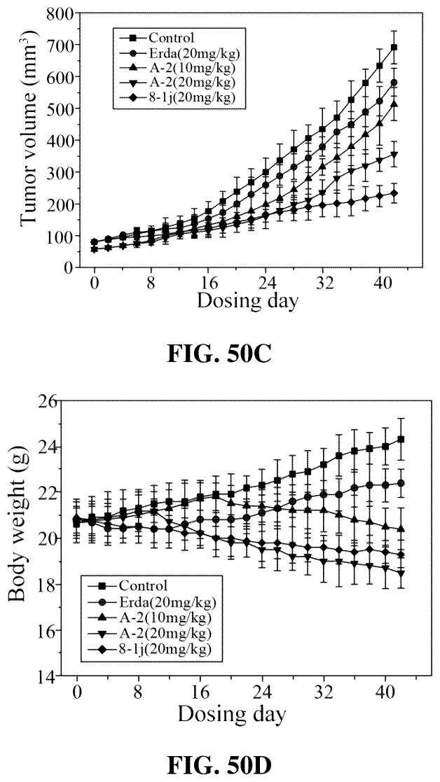

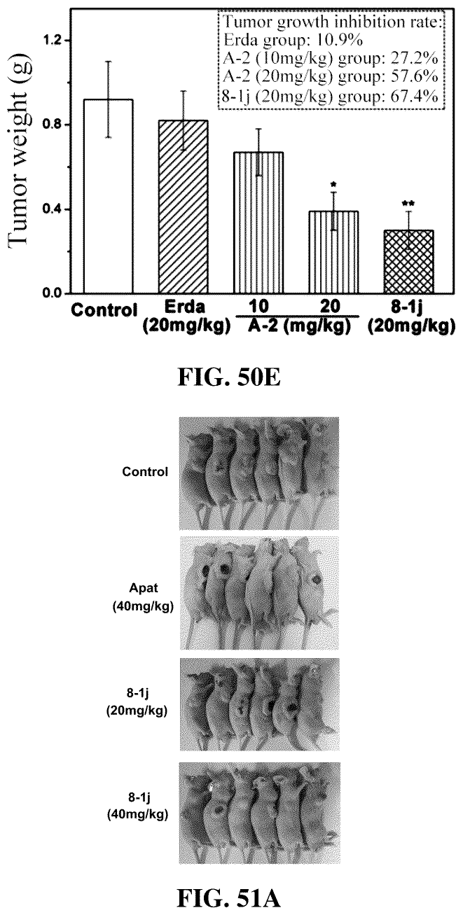

Experimental method: bladder cancer J82 cells in logarithmic growth phase were trypsinized, collected, and then centrifuged, and resuspended in serum-free DMEM medium after discarding an original medium. Cell density was adjusted, and 0.2 mL cell suspension was subcutaneously inoculated into 5-week-old BALB/c-nu mice (sex-balanced, 18-20 g) under a laminar flow clean bench. Post-inoculation, bleeding at injection sites was monitored. When tumors reached 50-80 mm³, nude mice with good tumor growth status were randomized into 5 groups (n=6/group) for intraperitoneal administration. Tumor volume and body weight were recorded on Day 0. Group designations: (1) Vehicle control: normal saline; (2) Positive control: Erda solution (20 mg/kg); (3) Low-dose Compound 9g (A-2): 10 mg/kg; (4) High-dose Compound 9g (A-2): 20 mg/kg; (5) Compound 8-1j: 20 mg/kg. Dosing occurred every 2 d for 42 d. After administering the drug, mice were monitored for diarrhea, anorexia, lethargy, and allergic reactions (e.g., pruritus). The body weight of nude mice was measured every 2 days, and the long and short diameters of the tumors in nude mice were measured by using a vernier caliper, then the recording was finished. The calculation formulas for tumor volume and tumor growth inhibition value are respectively: i) Tumor volume=½×length×(width)²; ii) Tumor growth inhibition value (TGI)=[(Mean tumor weight_{negative control}−Mean tumor weightₜᵣₑₐₜₘₑₙₜ)/Mean tumor weight_{negative control}]×100%. The experimental results are shown in FIGS. 50A-50E.

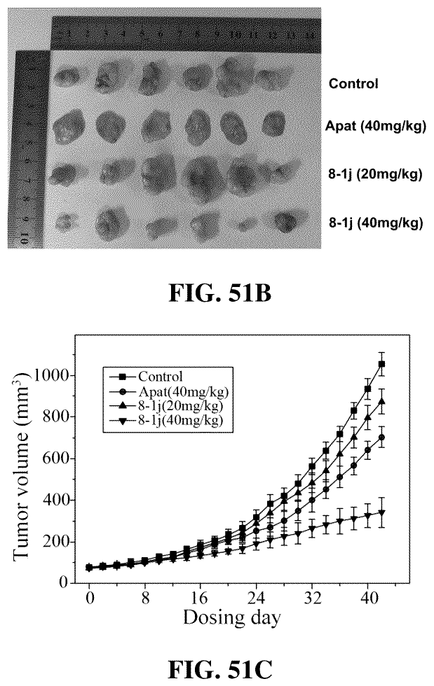

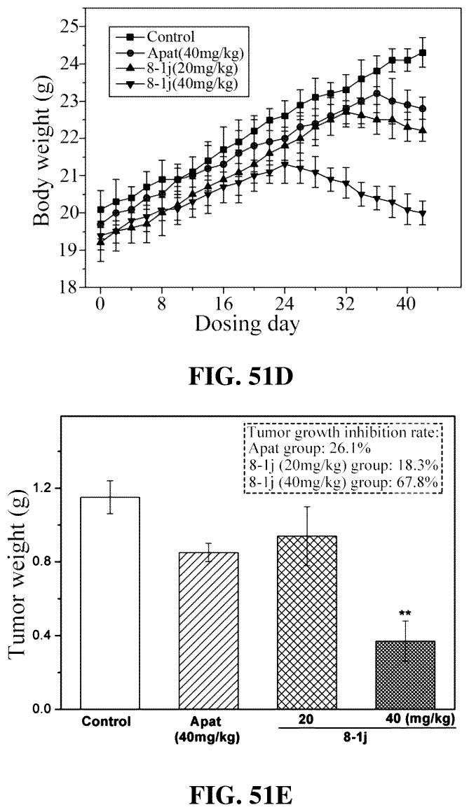

Construction of the human osteosarcoma 143B tumor-bearing nude mouse model followed similar procedures, with identical dosing frequency but administration route changed to oral gavage. Specifically: When tumors reached 60-90 mm³, mice were randomized into 4 groups (n=6). Group designations: (1) Vehicle control: normal saline; (2) Positive control: Apat suspension in normal saline (40 mg/kg); (3) Low-dose Compound 8-1j: 20 mg/kg; (4) High-dose Compound 8-1j: 40 mg/kg. Monitoring continued for 42 d. The experimental results are shown in FIGS. 51A-51E.

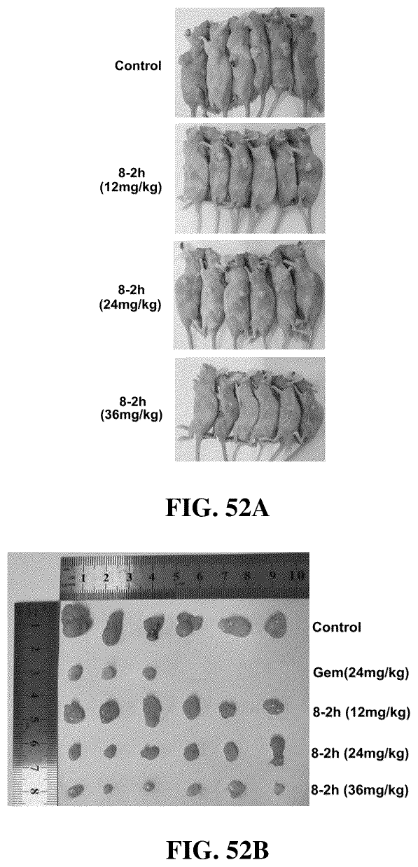

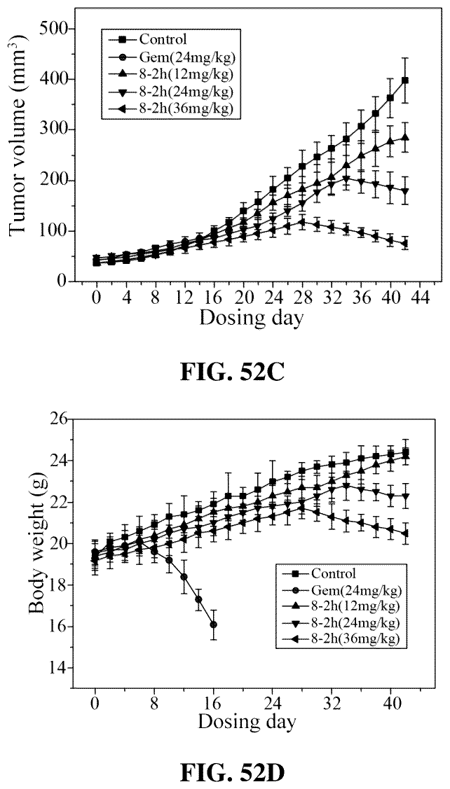

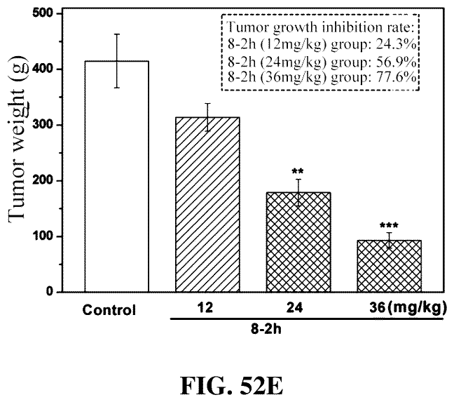

Construction of the human pancreatic cancer BxPC-3 tumor-bearing nude mouse model followed similar procedures, with identical dosing frequency and route, but featured different groupings/doses. Group designations: (1) Vehicle control: normal saline; (2) Positive control: gemcitabine solution in normal saline (24 mg/kg); (3) Low-dose Compound 8-2h: 12 mg/kg; (4) Mid-dose Compound 8-2h: 24 mg/kg; (5) High-dose Compound 8-2h: 36 mg/kg. Notably, in Group (2), 3 of 6 nude mice progressively died within 16 d. To ensure experiment continuity, this group was monitored for 16 d only, while others completed 42-d observation. The experimental results are shown in FIGS. 52A-52E.

Formulation of Injectable Preparations for Erda and 8-1j: Erda was weighed, dissolved in injection-grade N,N-dimethylacetamide (DMA), diluted with PEG400, and sonicated until dissolved. 0.9% normal saline was added thereto to obtain a solution, which was sterilized under UV light for 30 min. Administration volume was 10 mL/kg per mouse. The preparation contained PEG400 and 0.9% normal saline at a 3:1 volume ratio, with 2% (v/v) DMA. Compound 8-1j exhibits desirable solubility in the PEG400-0.9% normal saline mixture, requiring no DMA addition.

Formulation of Oral Gavage Preparations for Apat, 8-1j, and 8-2h: each compound was weighed, mixed with PEG400, and sonicated. 0.9% normal saline was added at a PEG400: 0.9% normal saline volume ratio of 3:1. A resulting mixture was sterilized under UV light for 30 min. Administration volume was 10 mL/kg per mouse.

Experimental results demonstrate potent tumor-suppressive effects of high-dose compounds 8-1j and 8-2h across 3 tumor-bearing nude mouse models. Asterisks denote: *p<0.05, **p<0.01, **p<0.001 vs. vehicle control. In the J82 tumor-bearing nude mouse model (FIGS. 50A-50E): tumor growth rates are significantly lower in the compound 8-1j group vs. vehicle control and equi-dose positive control. Mild weight loss occurs without anorexia, diarrhea, or pruritus, indicating desirable tolerability of the compound 8-1j at the above doses. Anti-tumor efficacy of the compound 8-1j surpassed that of 9g (A-2) at 20 mg/kg.

In the 143B osteosarcoma tumor-bearing nude mouse model (FIGS. 51A-51E): compound 8-1j (40 mg/kg) exhibits significantly stronger tumor inhibition than positive control Apat, which shows weak tumor inhibition activity. In the BxPC-3 tumor-bearing nude mouse model (FIGS. 52A-52E): Although the positive control gemcitabine shows acceptable in vitro proliferation inhibitory activity, it was found after oral gavage administration that the nude mice in this group exhibit significant gastrointestinal reactions: 50% mice (3 of total) manifest anorexia and emaciation after 4 doses, showing rapid weight decline culminating in progressive death of the 3 nude mice within 16 d. This group was unable to maintain continuous intragastric administration, indicating that gemcitabine may be unsuitable for oral gavage delivery. Due to abbreviated dosing, TGI was calculated only for 8-2h groups (FIG. 52E). Low, mid, and high doses of 8-2h groups show no toxicity; high-dose controls tumor growth by Day 28. After continued administration, the tumor significantly shrank, demonstrating that 8-2h exhibits potent antitumor activity and low toxicity in this model.

Additional formulation findings: Typical compounds 8-1j and 8-2h display superior solubility in co-solvents (e.g., PEG400+normal saline, PEG400+PBS, PEG400+5% dextrose injection) vs. 9g and positive control Erda. Compound 9g forms aggregates in normal saline/5% dextrose injection; I-8 has very low solubility in the above mentioned solvents, with only slight dissolution in DMF and DMSO. This enables injectable/perfusion formulations for 8-1j/8-2h, validating the imperative of 6-position quinazoline ring modifications.

The objects, technical solutions, and beneficial effects of the present disclosure are further described in detail through the above specific embodiments. It should be understood that the above are merely some specific embodiments of the present disclosure, but are not intended to limit the scope of the present disclosure.

What is claimed is:

1. A piperazinyl-modified quinazolinyl-aryl urea derivative or a pharmaceutically acceptable salt thereof, the piperazinyl-modified quinazolinyl-aryl urea derivative having a structure shown in 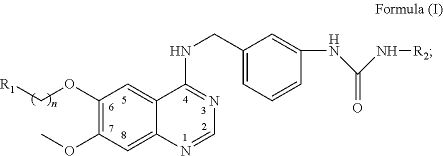
wherein,
n is in a range of 3 to 7;
R₁ is selected from the group consisting of 4-(2-morpholinoethyl)piperazin-1-yl and 4-((4-(pyridin-2-yl)piperazin-1-yl)methyl)piperidin-1-yl, wherein the 4-(2-morpholinoethyl)piperazin-1-yl has a structure shown in 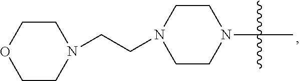 [SMILES: *N1CCN(CCN2CCOCC2)CC1]
and the 4-((4-(pyridin-2-yl)piperazin-1-yl)methyl)piperidin-1-yl has a structure shown in 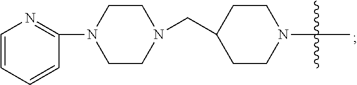 [SMILES: *N1CCC(CN2CCN(c3ccccn3)CC2)CC1]
and
R₂ is selected from the group consisting of phenyl, substituted phenyl, benzyl, and cycloalkyl; the substituted phenyl comprises a benzene ring and a substituent linked to the benzene ring, the substituent exhibits mono-substitution, identical multi-substitution, or different multi-substitution, and is at least one selected from the group consisting of alkyl, alkoxy, nitro, a halogen atom, and a halogenated group; and the cycloalkyl is selected from the group consisting of cyclopropyl, cyclobutyl, cyclopentyl, cyclohexyl, and cycloheptyl.

2. The piperazinyl-modified quinazolinyl-aryl urea derivative or the pharmaceutically acceptable salt thereof of claim 1, wherein the alkyl in the substituent is at least one selected from the group consisting of methyl, ethyl, propyl, isopropyl, butyl, tert-butyl, pentyl, and isopentyl; and/or,
the alkoxy in the substituent is at least one selected from the group consisting of methoxy, ethoxy, propoxy, isopropoxy, butoxy, and tert-butoxy; and/or,
the halogen atom in the substituent is at least one selected from the group consisting of a fluorine atom, a chlorine atom, a bromine atom, and an iodine atom, and the halogenated group is trifluoromethyl.

3. The piperazinyl-modified quinazolinyl-aryl urea derivative or the pharmaceutically acceptable salt thereof of claim 1, wherein R₂ is selected from the group consisting of phenyl, 2-methylphenyl, 3-methylphenyl, 4-methylphenyl, 2,4-dimethylphenyl, 3,5-dimethylphenyl, 2-ethylphenyl, 2,6-diethylphenyl, 4-isopropylphenyl, 4-tert-butylphenyl, 2-methoxyphenyl, 4-methoxyphenyl, 4-fluorophenyl, 2-chlorophenyl, 4-chlorophenyl, 2,4-dichlorophenyl, 3,4-dichlorophenyl, 2,4,6-trichlorophenyl, 4-(trifluoromethyl)phenyl, (3-trifluoromethyl-4-fluoro)phenyl, (3-trifluoromethyl-4-chloro)phenyl, (3,5-bis(trifluoromethyl))phenyl, benzyl, and cyclohexyl.

4. The piperazinyl-modified quinazolinyl-aryl urea derivative or the pharmaceutically acceptable salt thereof of claim 1, wherein the pharmaceutically acceptable salt is selected from the group consisting of a hydrochloride, a sulfate, a phosphate, a formate, an acetate, a nitrate, a benzoate, a citrate, a succinate, a palmitate, a stearate, a besylate, a tartrate, a maleate, an oxalate, a malate, a lactate, a tosylate, a mesylate, an ethanesulfonate, a triflate, and a trifluoroacetate.

5. A method for preparing the piperazinyl-modified quinazolinyl-aryl urea derivative or the pharmaceutically acceptable salt thereof of claim 1, comprising the following steps:
subjecting an intermediate 2 to demethylation with a demethylation reagent under heating to obtain an intermediate 3; wherein
the demethylation reagent comprises concentrated sulfuric acid and L-methionine;
the intermediate 2 has a structure shown in 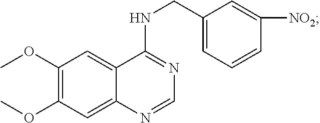 [SMILES: COc1cc2ncnc(NCc3cccc([N+](=O)[O-])c3)c2cc1OC]
and
the intermediate 3 has a structure shown in 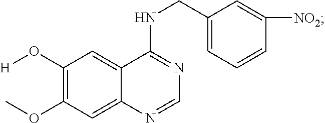 [SMILES: COc1cc2ncnc(NCc3cccc([N+](=O)[O-])c3)c2cc1O]
subjecting the intermediate 3, a haloalkyl compound, and K₂CO₃ to a first substitution reaction under an inert gas atmosphere while heating to obtain an intermediate 4; wherein
the haloalkyl compound has a structure shown in 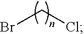
and
the intermediate 4 has a structure shown in 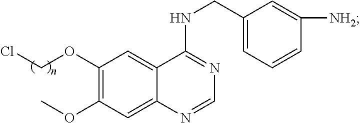
subjecting the intermediate 4, a substituted compound, K₂CO₃, and sodium iodide to a second substitution reaction under an inert gas atmosphere while heating to obtain an intermediate 5; wherein
the substituted compound has a structure selected from the group consisting of 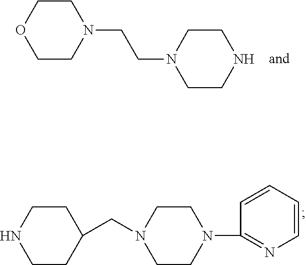 [SMILES: C1CN(CCN2CCOCC2)CCN1 ; c1ccc(N2CCN(CC3CCNCC3)CC2)nc1]
and
the intermediate 5 has a structure shown in 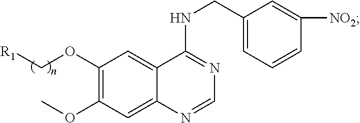
subjecting the intermediate 5 to nitro reduction with a reducing agent under heating to obtain an intermediate 6; wherein
the intermediate 6 has a structure shown in 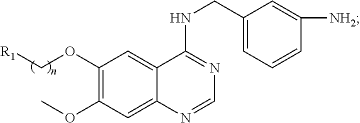
and
subjecting the intermediate 6 and an intermediate 7 to a reaction in the presence of a catalyst to obtain the piperazinyl-modified quinazolinyl-aryl urea derivative or the pharmaceutically acceptable salt thereof; wherein
the intermediate 7 has a structure shown in R₂—N═C═O; and
wherein n in the haloalkyl compound, the intermediate 4, the intermediate 5, and the intermediate 6 corresponds to n in the Formula (I); R₁ in the intermediate 5 and the intermediate 6 corresponds to R₁ in the Formula (I); and R₂ in the intermediate 7 corresponds to R₂ in the Formula (I).

6. The method for preparing the piperazinyl-modified quinazolinyl-aryl urea derivative or the pharmaceutically acceptable salt thereof of claim 5, wherein a molar ratio of the intermediate 2, the L-methionine, and the concentrated sulfuric acid is in a range of 1:(1.2-2.5):(12-25), and the demethylation is conducted at a temperature of 90° C. to 120° C. for 8 hours to 16 hours; and/or,
the reducing agent is selected from the group consisting of a combination of an activated iron powder and a 5 wt % to 20 wt % aqueous ammonium chloride solution and a combination of the activated iron powder and a 10 wt % to 20 wt % dilute hydrochloric acid, a molar ratio of the intermediate 5, the activated iron powder, and ammonium chloride is in a range of 1:(5-10):(1-2.5), a reaction system for the nitro reduction further comprises methanol as a solvent, the nitro reduction is conducted at a temperature of 55° C. to 65° C. for 8 hours to 16 hours, and the activated iron powder is prepared by treating a reduced iron powder with a 10 wt % to 20 wt % aqueous hydrochloric acid solution.

7. The method for preparing the piperazinyl-modified quinazolinyl-aryl urea derivative or the pharmaceutically acceptable salt thereof of claim 5, wherein under a condition that the substituted compound has the structure shown in 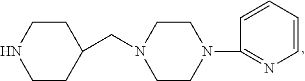 [SMILES: c1ccc(N2CCN(CC3CCNCC3)CC2)nc1]
the substituted compound is prepared by a process comprising the following steps:
subjecting tert-butyl 4-(bromomethyl)piperidin-1-carboxylate, 1-(2-pyridyl)piperazine, the K₂CO₃, and the sodium iodide to a first reaction under heating to obtain tert-butyl 4-((4-(pyridin-2-yl)piperazin-1-yl)methyl)piperidin-1-carboxylate; and
subjecting the tert-butyl 4-((4-(pyridin-2-yl)piperazin-1-yl)methyl)piperidin-1-carboxylate and concentrated hydrochloric acid to a second reaction, and then subjecting a resulting reaction product to basification to a pH value of 10 to obtain the substituted compound.

8. A drug, comprising the piperazinyl-modified quinazolinyl-aryl urea derivative or the pharmaceutically acceptable salt thereof of claim 1, or a preparation of the piperazinyl-modified quinazolinyl-aryl urea derivative in combination with an auxiliary material.

9. The drug of claim 8, wherein the preparation comprises a pharmaceutically acceptable excipient and/or a pharmaceutically acceptable additive; and/or,
the preparation is selected from the group consisting of an oral preparation, an injectable preparation, a topical preparation, and an intracavitary administration preparation.

10. A method for treating a tumor, comprising administrating a therapeutic effective amount of the piperazinyl-modified quinazolinyl-aryl urea derivative or the pharmaceutically acceptable salt thereof of claim 1 to a subject in need thereof,
wherein the tumor is at least one selected from the group consisting of bladder cancer, osteosarcoma, pancreatic cancer, breast cancer, cervical cancer, non-small cell lung cancer (NSCLC), ovarian cancer, hepatoblastoma, liver cancer, prostate cancer, renal carcinoma, and colorectal cancer.

11. The method for preparing the piperazinyl-modified quinazolinyl-aryl urea derivative or the pharmaceutically acceptable salt thereof of claim 5, wherein the alkyl in the substituent is at least one selected from the group consisting of methyl, ethyl, propyl, isopropyl, butyl, tert-butyl, pentyl, and isopentyl; and/or,
the alkoxy in the substituent is at least one selected from the group consisting of methoxy, ethoxy, propoxy, isopropoxy, butoxy, and tert-butoxy; and/or,
the halogen atom in the substituent is at least one selected from the group consisting of a fluorine atom, a chlorine atom, a bromine atom, and an iodine atom, and the halogenated group is trifluoromethyl.

12. The method for preparing the piperazinyl-modified quinazolinyl-aryl urea derivative or the pharmaceutically acceptable salt thereof of claim 5, wherein R₂ is selected from the group consisting of phenyl, 2-methylphenyl, 3-methylphenyl, 4-methylphenyl, 2,4-dimethylphenyl, 3,5-dimethylphenyl, 2-ethylphenyl, 2,6-diethylphenyl, 4-isopropylphenyl, 4-tert-butylphenyl, 2-methoxyphenyl, 4-methoxyphenyl, 4-fluorophenyl, 2-chlorophenyl, 4-chlorophenyl, 2,4-dichlorophenyl, 3,4-dichlorophenyl, 2,4,6-trichlorophenyl, 4-(trifluoromethyl)phenyl, (3-trifluoromethyl-4-fluoro)phenyl, (3-trifluoromethyl-4-chloro)phenyl, (3,5-bis(trifluoromethyl))phenyl, benzyl, and cyclohexyl.

13. The method for preparing the piperazinyl-modified quinazolinyl-aryl urea derivative or the pharmaceutically acceptable salt thereof of claim 5, wherein the pharmaceutically acceptable salt is selected from the group consisting of a hydrochloride, a sulfate, a phosphate, a formate, an acetate, a nitrate, a benzoate, a citrate, a succinate, a palmitate, a stearate, a besylate, a tartrate, a maleate, an oxalate, a malate, a lactate, a tosylate, a mesylate, an ethanesulfonate, a triflate, and a trifluoroacetate.

14. The drug of claim 8, wherein the alkyl in the substituent is at least one selected from the group consisting of methyl, ethyl, propyl, isopropyl, butyl, tert-butyl, pentyl, and isopentyl; and/or,
the alkoxy in the substituent is at least one selected from the group consisting of methoxy, ethoxy, propoxy, isopropoxy, butoxy, and tert-butoxy; and/or,
the halogen atom in the substituent is at least one selected from the group consisting of a fluorine atom, a chlorine atom, a bromine atom, and an iodine atom, and the halogenated group is trifluoromethyl.

15. The drug of claim 8, wherein R₂ is selected from the group consisting of phenyl, 2-methylphenyl, 3-methylphenyl, 4-methylphenyl, 2,4-dimethylphenyl, 3,5-dimethylphenyl, 2-ethylphenyl, 2,6-diethylphenyl, 4-isopropylphenyl, 4-tert-butylphenyl, 2-methoxyphenyl, 4-methoxyphenyl, 4-fluorophenyl, 2-chlorophenyl, 4-chlorophenyl, 2,4-dichlorophenyl, 3,4-dichlorophenyl, 2,4,6-trichlorophenyl, 4-(trifluoromethyl)phenyl, (3-trifluoromethyl-4-fluoro)phenyl, (3-trifluoromethyl-4-chloro)phenyl, (3,5-bis(trifluoromethyl))phenyl, benzyl, and cyclohexyl.

16. The drug of claim 8, wherein the pharmaceutically acceptable salt is selected from the group consisting of a hydrochloride, a sulfate, a phosphate, a formate, an acetate, a nitrate, a benzoate, a citrate, a succinate, a palmitate, a stearate, a besylate, a tartrate, a maleate, an oxalate, a malate, a lactate, a tosylate, a mesylate, an ethanesulfonate, a triflate, and a trifluoroacetate.
